SEQUENCE LISTING (10 sequences)

SEQ ID NO: 1 - protein, 120 aa, Artificial Sequence
  Synthetic heavy chain variable domain
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 2 - protein, 111 aa, Artificial Sequence
  Synthetic light chain variable domain
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLG

SEQ ID NO: 3 - protein, 251 aa, Artificial Sequence
  Synthetic single-chain antibody
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 4 - protein, 252 aa, Artificial Sequence
  Synthetic single-chain antibody
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 5 - protein, 11 aa, Artificial Sequence
  CDRL1
QASEIIHSWLA

SEQ ID NO: 6 - protein, 7 aa, Artificial Sequence
  CDRL2
LASTLAS

SEQ ID NO: 7 - protein, 12 aa, Artificial Sequence
  CDRL3
QNVYLASTNGAN

SEQ ID NO: 8 - protein, 11 aa, Artificial Sequence
  CDRH1
GFSLTDYYYMT

SEQ ID NO: 9 - protein, 16 aa, Artificial Sequence
  CDRH2
FIDPDDDPYYATWAKG

SEQ ID NO: 10 - protein, 11 aa, Artificial Sequence
  CDRH3
GDHNSGWGLDI

(19) United States
(12) Patent Application Publication
(10) Pub. No.: US 2023/0416351 A1
(43) Pub. Date: Dec. 28, 2023

(54) VEGF ANTAGONIST FOR USE IN METHODS FOR TREATING OCULAR DISEASES

(71) Applicant: NOVARTIS AG, Basel (CH)

(72) Inventors: Andreas Clemens, Au in Breisgau (DE); Martina Junge, Nürnberg (DE); Jeffrey David Kearns, Arlington, MA (US); Ufuk Olgac, Zurich (CH); Jessica Voegeler, Parsberg (DE)

(21) Appl. No.: 18/038,374

(22) PCT Filed: Nov. 24, 2021
(86) PCT No.: PCT/IB2021/060905

§ 371 Date: May 23, 2023

Related U.S. Application Data
(60) Provisional application No. 63/270,864, filed on Oct. 22, 2021.

(30) Foreign Application Priority Data
Nov. 25, 2020 (EP) 20209685.5

(51) Int. Cl. C07K 16/22; A61P 27/02

(52) U.S. Cl. CPC C07K 16/22; A61P 27/02; C07K 2317/622; C07K 2317/55

(57) ABSTRACT

The invention relates to methods for treating ocular disease with a VEGF antagonist. In particular, the invention relates to methods for treating ocular disease, e.g., neovascular age-related macular degeneration (nAMD), in a patient, the method comprising administering to the patient one initial dose of a VEGF antagonist, e.g., brolucizumab, followed by a maintenance regimen of additional doses of the VEGF antagonist administered in an administration interval of at least 8 weeks. In particular, the invention relates to methods for treating ocular disease, in particular neovascular age-related macular degeneration (nAMD), in a patient pretreated with one or more doses of a VEGF antagonist B, the method comprising administering to the patient an initial dose of a VEGF antagonist A followed by one or more additional doses of a VEGF antagonist A in an administration interval according to a maintenance regimen of the VEGF antagonist A for the treatment of the ocular disease.

### SEQUENCE LISTING

The instant application contains a Sequence Listing which has been submitted electronically in ASCII format and is hereby incorporated by reference in its entirety. Said ASCII copy, created on Nov. 18, 2020, is named PAT058982_SEQ_LISTING_ST25.txt and is 8 KB in size.

### FIELD

The invention relates to methods for treating ocular disease with a VEGF antagonist. In particular, the invention relates to methods for treating ocular disease, e.g., neovascular age-related macular degeneration (nAMD), in a patient, the method comprising administering to the patient one initial dose of a VEGF antagonist, e.g., brolucizumab, followed by a maintenance regimen of additional doses of the VEGF antagonist administered in an administration interval of at least 8 weeks. In particular, the invention relates to methods for treating ocular disease, in particular neovascular age-related macular degeneration (nAMD), in a patient pretreated with one or more doses of a VEGF antagonist B, the method comprising administering to the patient an initial dose of a VEGF antagonist A followed by one or more additional doses of a VEGF antagonist A in an administration interval according to a maintenance regimen of the VEGF antagonist A for the treatment of the ocular disease.

### BACKGROUND

Age-related macular degeneration (AMD) is the leading cause of severe vision loss in people affecting 10%-13% of individuals over the age of 65 in North America, Europe, and Australia (Kawasaki 2010, Rein et al., Arch Ophthalmol. 2009; 127:533-40, Smith 2001). Genetic, environmental and health factors play an important role in the pathogenesis of the disease.

AMD is classified into 2 clinical subtypes: the non-neovascular (atrophic) or dry form and the neovascular (exudative) or wet form (Ferris et al., Arch Ophthalmol. 1984; 102:1640-2, Lim et al., Lancet. 2012; 379:1728-38, Miller et al., Am J Ophthalmol. 2013; 155:1-35). Neovascular AMD (nAMD) is characterized by the growth of abnormal new blood vessels (neovascularization) under the retinal pigment epithelium (RPE) or subretinal space from the subjacent choroid, termed choroidal neovascularization (CNV) (Ferris et al., Arch Ophthalmol. 1984; 102:1640-2). These newly formed vessels have an increased likelihood to leak blood and serum, damaging the retina by stimulating inflammation and scar tissue formation. This damage to the retina results in progressive, severe, and irreversible vision loss (Shah et al., Am J Ophthalmol. 2007; 143:83-89, Shah et al., Am J Ophthalmol. 2009; 116:1901-07). Without treatment, most affected eyes will have poor central vision (20/200) within 12 months (TAP 2003). Although the neovascular form of the disease is only present in about 10% of all AMD cases, it accounted for approximately 90% of the severe vision loss from AMD prior to the introduction of anti-vascular endothelial growth factor (VEGF) treatments (Ferris et al., Am J Ophthalmol. 1983; 118:132-51, Sommer et al., N Engl J Med. 1991; 14:1412-17, Wong et al., Ophthalmology. 2008; 115:116-26).

VEGF has been shown to be elevated in patients with nAMD and is thought to play a key role in the neovascularization process (Spilsbury et al., Am J Pathol. 2000; 157:135-44). The use of intravitreal (IVT) pharmacotherapy targeting VEGF has significantly improved visual outcomes in patients with nAMD (Bloch et al., Am J Ophthalmol. 2012; 153:209-13, Campbell et al., Arch Ophthalmol. 2012; 130:794-5). Anti-VEGF treatments, such as ranibizumab (LUCENTIS©), aflibercept (EYLEA®), and brolucizumab (Beovu®), inhibit VEGF signaling pathways and have been shown to halt the growth of neovascular lesions and resolve retinal edema.

In two Phase 3 studies of ranibizumab, with monthly dosing regimens, approximately 95% of ranibizumab treated subjects experienced stabilization of vision (defined as a loss of fewer than 15 ETDRS letters) or improvement in vision at 12 months compared with 62% and 64% in the control groups (Rosenfeld et al., N Engl J Med. 2006; 355:1419-31, Brown et al., N Engl J Med. 2006; 355:1432-44). Twenty-five to 40% of subjects in the ranibizumab groups gained ≥15 letters at 12 months compared with 5-6% in the 2 control groups. On average, ranibizumab treated subjects gained 7-11 letters of vision after 12 months, whereas control subjects lost an average of approximately 10 letters. This gain in visual acuity was essentially maintained during the second year of both Phase 3 studies while vision, on average, continued to decline in the control group. The visual acuity benefits, which indicate a suspension of nAMD rather than a slowdown of its progression, were supported by corresponding effects on lesion anatomy and subject reported outcomes. The latter demonstrated statistically and clinically meaningful improvements in near activities, distance activities, and vision specific dependency as measured by the National Eye Institute Visual Functioning Questionnaire—25 (VFQ-25).

In two parallel Phase 3 trials of aflibercept, treatment naïve subjects with nAMD were randomized to 2 doses (0.5 and 2.0 mg) and 2 regimen (every 4 weeks and every 8 weeks with 2.0 mg) or the control arm (ranibizumab 0.5 mg every 4 weeks). At 52 weeks, all aflibercept groups, independent of doses and regimen, were noninferior to the ranibizumab group with equal maintenance of vision in 95% of eyes (Heier et al., Ophthalmology. 2012; 119:2537-48). In the 2 mg aflibercept every 4 weeks group, there was a mean BCVA improvement of 9.3 letters and in the 2 mg aflibercept every 8 weeks group there was an improvement of 8.4 letters compared to the control group which had a mean improvement of 8.7 letters. In the second year of the study subjects were switched to a capped pro-re-nata (PRN) regimen. The proportion of subjects who maintained BCVA ranged between 91% and 92% for all groups. Mean BCVA improvements ranged from 7.9 (ranibizumab 0.5 mg every 4 weeks), 7.6 (aflibercept 2 mg every 4 weeks and every 8 weeks) to 6.6 (aflibercept 0.5 mg). Over all groups, a mean loss of 0.8-1.7 letters was seen after switching from a fixed to a capped PRN regimen. The retreatment frequency was similar between aflibercept and ranibizumab arms during the capped PRN year, with 4.1 injections for the aflibercept 2 mg every 4 weeks arm, 4.2 injections for the aflibercept 2 mg every 8 weeks arm and 4.7 for the ranibizumab 0.5 mg every 4 weeks arm (Schmidt-Erfurth et al., Br J Ophthalmol 2014; 98:1144-1167 2014; 98:1144-1167).

Two similarly designed phase 3 trials (HAWK and HARRIER) compared brolucizumab, a single-chain antibody fragment that inhibits vascular endothelial growth factor-A, with aflibercept to treat neovascular age-related macular degeneration (nAMD) (Dugel et al., Ophthalmology, Volume 127, Issue 1, January 2020, Pages 72-84). The HAWK (NCT02307682) and HARRIER (NCT02434328) studies, which were 2-year, double-masked, multicenter Phase 3 studies investigating the efficacy of brolucizumab versus aflibercept in treatment naïve patients with nAMD. Patients were randomized to intravitreal brolucizumab 3 mg (HAWK only) or 6 mg or aflibercept 2 mg. After loading with 3 monthly injections, brolucizumab-treated eyes received an injection every 12 weeks (q12w) and were interval adjusted to every 8 weeks (q8w) if disease activity was present; aflibercept-treated eyes received q8w dosing. Brolucizumab was noninferior to aflibercept in visual function at Week 48, and >50% of brolucizumab 6 mg-treated eyes were maintained on q12w dosing interval through Week 48. Anatomic outcomes favored brolucizumab over aflibercept. Clinical treatment-effect data from patients receiving brolucizumab 6 mg in the HAWK and HARRIER studies were also compared with modelled placebo data (Agostini et al., Curr Eye Res, 2020 October; 45(10):1298-1301). Compared with a modelled placebo, brolucizumab treatment was associated with an overall best corrected visual acuity gain of approximately 22 Early Treatment Diabetic Retinopathy Study (ETDRS) letters at Week 48 and 28 letters at Week 96.

Currently marketed anti-VEGF treatments typically start with a loading phase of 3 monthly doses, followed by maintenance dosing, either with fixed (e.g. every 4 or 8 weeks or every 12 weeks) or individualized treatment intervals, based on pro re nata (PRN) or Treat-and-Extend (T&E) concepts (Wykoff et al., 2018). Monthly treatment or treatment every 2 months poses significant burden not only for the generally older patients but also for their caregivers and physicians. Also, although the treatments have proven to have a positive benefit/risk ratio, they are not without risk. Each injection carries with it the possibility of pain, sub-conjunctival hemorrhage, vitreous hemorrhage, retinal tear, retinal detachment, iatrogenic cataract, and endophthalmitis (Ohr et al., Expert Opin. Pharmacother. 2012; 13:585-591), as well as a sustained rise in intraocular pressure (IOP) with serial injections of anti-VEGF agents (Tseng et al., J Glaucoma. 2012; 21:241-47). Additionally, even with monthly IVT injections, 60-70% of patients gain less than 15 letters of visual acuity. In ranibizumab and aflibercept trials, both interventional (e.g. TREND (Silva et al., Ophthalmology; 2018, 125:57-65), ALTAIR (Bayer AG, 2017, Package leaflet Eylea®—Germany)) and real life studies (prospective non-interventional trials, e.g. OCEAN (Voegeler and Mueller, Non-interventional Final Study Report CRFB002ADE18, 2017)), when extended to q6w or longer treatment intervals, a number of patients still showed persistent fluid, although initial functional and anatomical response after the loading phase (three initial injections) was seen. For these patients, a longer lasting anti-VEGF agent like brolucizumab (e.g. maintenance dosing every 8 or 12 weeks) may lead to optimized fluid, and disease control i.e. sustained functional and anatomical response, respectively; which overall might result in improved patient care (e.g. less frequent visits, reduced treatment burden).

Despite the treatment success of existing anti-VEGFs, there remains a need for further treatment options to improve response rate and/or reduce resource use and injection frequency in patients with nAMD. Current dosing regimens are developed for naïve patients and include a loading phase of three monthly doses, which imposes a high treatment burden on a patient during the loading phase. There is a medical need to optimize the dosing regimen for anti-VEGF in nAMD patients to improve patient care (e.g. less frequent visits, reduced treatment burden). There is also a medical need to optimize the dosing regimen for anti-VEGF pretreated nAMD patients switching to a different anti-VEGF agent, e.g., the anti-VEGF agent achieving a greater gain in visual acuity and/or better anatomical outcomes in a higher number of patients and/or having a prolonged therapeutic effect.

### SUMMARY

The invention provides a method for treating ocular disease, e.g., neovascular age-related macular degeneration (nAMD), in a patient, e.g., a naïve patient, the method comprising administering to the patient an initial dose of a VEGF antagonist, e.g., brolucizumab, followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

In one aspect, the present invention provides methods for treating ocular disease, e.g., nAMD, in a patient, the method comprising administering to the patient an initial dose of a VEGF antagonist, e.g., brolucizumab, followed by a second dose at least 8 weeks after the initial dose, e.g., 8, 9, 10, 11, 12, 13, 14, 15, 16, 17, 18, 19, 20, 21, 22, 23, or 24 weeks.

In one aspect, the present invention provides methods for treating ocular disease, e.g., nAMD, in a patient, the method comprising administering to the patient an initial dose of a VEGF antagonist, e.g., brolucizumab, followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

In one aspect, the present invention provides methods for treating ocular disease in a patient, the method comprising administering to the patient one initial dose of a VEGF antagonist followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

In a further aspect, the present invention provides a VEGF antagonist for use as a medicament for treating ocular disease in a patient, wherein the VEGF antagonist is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present invention provides a VEGF antagonist for use as a medicament for treating ocular disease in a patient, wherein the VEGF antagonist is administered to the patient as an initial dose followed by one or more additional doses, wherein the one or more additional doses of the VEGF antagonist are administered at least 8 weeks after the initial dose and each of the one or more additional doses after the initial dose are administered in an administration interval of at least 8 weeks.

In another aspect, the present invention provides a VEGF antagonist for use as a medicament for treating ocular disease in a patient, wherein the VEGF antagonist is administered to the patient as one initial dose followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

In a further aspect, the present invention provides a pharmaceutical composition comprising a VEGF antagonist for use as a medicament for treating ocular disease in a patient, wherein the pharmaceutical composition is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present invention provides a pharmaceutical composition comprising a VEGF antagonist for use as a medicament for treating ocular disease in a patient, wherein the pharmaceutical composition is administered to the patient as an initial dose followed by one or more additional doses, wherein the one or more additional doses of the pharmaceutical composition are administered at least 8 weeks after the initial dose and each of the one or more additional doses after the initial dose are administered in an administration interval of at least 8 weeks.

In another aspect, the present invention provides a pharmaceutical composition comprising a VEGF antagonist for use as a medicament for treating ocular disease in a patient, wherein the pharmaceutical composition is administered to the patient as one initial dose followed by a maintenance regimen of additional doses of the pharmaceutical composition administered in an administration interval of at least 8 weeks, e.g., 8, 9, 10, 11, 12, 13, 14, 15, 16, 17, 18, 19, 20, 21, 22, 23, or 24 week intervals.

In a further aspect, the present invention provides use of a VEGF antagonist for the manufacture of a medicament for treating ocular disease in a patient, the use comprising administering to the patient an initial dose of the VEGF antagonist followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present invention provides use of a VEGF antagonist for the manufacture of a medicament for treating ocular disease in a patient, the use comprising administering to the patient an initial dose of the VEGF antagonist followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

In another aspect, the present invention provides use of a VEGF antagonist for the manufacture of a medicament for treating ocular disease in a patient, the use comprising administering to the patient one initial dose of the VEGF antagonist followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

In some embodiments, the methods or the uses of the present invention are suitable for the treatment of a naïve patient. In some embodiments, the methods or the uses of the present invention are suitable for the treatment of a pretreated patient, e.g., a patient pretreated with one or more doses of a VEGF antagonist different from the VEGF antagonist administered according to the methods and uses of the present invention.

The invention further provides a method of administering a therapeutic VEGF antagonist A (e.g., brolucizumab) for treating ocular disease, e.g., neovascular age-related macular degeneration (nAMD), to a patient pretreated with one or more doses of a VEGF antagonist B (e.g., aflibercept or ranibizumab). In one aspect, the present invention provides methods for treating ocular disease, e.g., nAMD, in a patient that has previously received one or more doses of a VEGF antagonist B (e.g., aflibercept, ranibizumab) or in a patient pretreated with one or more doses of a VEGF antagonist B (e.g., aflibercept or ranibizumab), the method comprises administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks (at least 8 weeks) and/or as individualized by a physician based on a disease activity assessment.

In one aspect, the present invention provides methods for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), the method comprises administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease.

In a further aspect, the present invention provides a VEGF antagonist A (e.g., brolucizumab) for use as a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the VEGF antagonist A (e.g., brolucizumab) is administered to the patient as an initial dose followed by one or more additional doses, in particular wherein said one or more additional doses of the VEGF antagonist A are administered in an administration interval of no less than about 8 weeks (at least 8 weeks) and/or as individualized by a physician based on a disease activity assessment.

In a further aspect, the present invention provides a VEGF antagonist A (e.g., brolucizumab) for use as a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the VEGF antagonist A (e.g., brolucizumab) is administered to the patient as an initial dose followed by one or more additional doses in an administration interval according to a maintenance regimen of the VEGF antagonist A for the treatment of the ocular disease.

In a further aspect, the present invention provides a pharmaceutical composition comprising a VEGF antagonist A for use as a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the pharmaceutical composition comprising a VEGF antagonist A (e.g., brolucizumab) is administered to the patient as an initial dose followed by one or more additional doses, in particular wherein said one or more additional doses of the VEGF antagonist A are administered in an administration interval of no less than about 8 weeks (at least 8 weeks) and/or as individualized by a physician based on a disease activity assessment.

In a further aspect, the present invention provides a pharmaceutical composition comprising a VEGF antagonist A for use as a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the pharmaceutical composition comprising a VEGF antagonist A (e.g., brolucizumab) is administered to the patient as an initial dose followed by one or more additional doses in an administration interval according to a maintenance regimen of the VEGF antagonist A for the treatment of the ocular disease.

In one aspect, the present invention provides use of a VEGF antagonist A (e.g., brolucizumab) for the manufacture of a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A, in particular wherein said one or more additional doses of the VEGF antagonist A are administered in an administration interval of no less than about 8 weeks (at least 8 weeks) and/or as individualized by a physician based on a disease activity assessment.

In one aspect, the present invention provides use of a VEGF antagonist A (e.g., brolucizumab) for the manufacture of a medicament for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A in an administration interval according to a maintenance regimen of the VEGF antagonist A for the treatment of the ocular disease.

In one aspect, the present invention provides use of a VEGF antagonist A (e.g., brolucizumab) for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A, in particular wherein said one or more additional doses of the VEGF antagonist A are administered in an administration interval of no less than about 8 weeks (at least 8 weeks) and/or as individualized by a physician based on a disease activity assessment.

In one aspect, the present invention provides use of a VEGF antagonist A (e.g., brolucizumab) for treating ocular disease, e.g., nAMD, in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprising administering to the patient an initial dose of a VEGF antagonist A (e.g., brolucizumab) followed by one or more additional doses of a VEGF antagonist A in an administration interval according to a maintenance regimen of the VEGF antagonist A for the treatment of the ocular disease.

In one embodiment, the method or the use or the VEGF antagonist A for use of the present invention comprises discontinuing treatment with the VEGF antagonist B. In one embodiment, the method or the use or the VEGF antagonist A for use of the present invention comprises administering to the patient the VEGF antagonist A in replacement of the VEGF antagonist B and no additional or alternative VEGF antagonists are administered to the patient during administration of the VEGF antagonist A. In one embodiment, the present disclosure provides methods or uses for treating ocular disease, e.g., nAMD, in a patient switched from a therapy with the VEGF antagonist B (e.g., aflibercept or ranibizumab) to a therapy with the VEGF antagonist A (e.g., brolucizumab).

In certain embodiments, the patient pretreated with one or more doses of the VEGF antagonist B (e.g., aflibercept or ranibizumab) or the patient that has previously received one or more doses of the VEGF antagonist B (e.g., aflibercept or ranibizumab) has suboptimal anatomically controlled ocular disease, e.g., nAMD, or has presence of ocular disease activity, e.g., nAMD disease activity, or was intolerant to the treatment with the VEGF antagonist B.

In some embodiments, the initial dose of the VEGF antagonist A (e.g., brolucizumab) is administered to the patient between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B (e.g., aflibercept or ranibizumab) was administered to the patient. In some embodiments, the period of time between discontinuing treatment with the VEGF antagonist B (e.g., aflibercept or ranibizumab) and start of administration of the VEGF antagonist A (e.g., brolucizumab) is about 4 to 6 weeks, about 4 to 8 weeks, or about 4 to 10 weeks, or about 4 to 12 weeks, or about 4 to 16 weeks, or about 4 to 18 weeks, or about 4 to 21 weeks, or about 4 to 24 weeks, in particular about 6 to 8 weeks, or about 6 to 10 weeks, or about 6 to 12 weeks, or about 6 to 16 weeks, or about 6 to 18 weeks, or about 6 to 21 weeks, or about 6 to 24 weeks.

In some embodiments, the initial dose of the VEGF antagonist A (e.g., brolucizumab) is followed by one or more doses of the VEGF antagonist A in an administration interval as individualized by a physician based on a disease activity assessment. In some embodiments, the initial dose of the VEGF antagonist A (e.g., brolucizumab) is followed by one or more doses of the VEGF antagonist A in an administration interval of no less than about 8 weeks, e.g., no less than about 12 weeks, e.g., in an administration interval between about ≥8 and about ≤24 weeks, e.g., in an administration interval between about ≥8 and about ≤12 weeks. In some embodiments, the initial dose of the VEGF antagonist A (e.g., brolucizumab) is followed by one or more doses of the VEGF antagonist A in an administration interval according to a maintenance regimen of the VEGF antagonist A for the treatment of the ocular disease (e.g., nAMD), e.g., without a loading phase of the VEGF antagonist A.

In certain embodiments, a dosing frequency or an administration interval of the VEGF antagonist A is adjusted based on the outcome of disease activity assessments, for example using pre-defined visual and anatomic criteria. In one embodiment, a dosing frequency or an administration interval of the VEGF antagonist A (e.g., brolucizumab) can be adjusted by decreasing the dosing interval from once every 24 weeks (q24w) to once every 18 weeks (q18w). In one embodiment, a dosing frequency or an administration interval of the VEGF antagonist A (e.g., brolucizumab) can be adjusted by decreasing the dosing interval from once every 18 weeks (q18w) to once every 12 weeks (q12w). In one embodiment, a dosing frequency or an administration interval of the VEGF antagonist A (e.g., brolucizumab) can be adjusted by decreasing the dosing interval from once every 12 weeks (q12w) to once every 8 weeks (q8w) or to once every 6 weeks (q6w) based on a disease activity assessment at any scheduled treatment visit. In another embodiment, a dosing frequency or an administration interval of the VEGF antagonist A (e.g., brolucizumab) can be adjusted by increasing the dosing interval from once every 6 weeks (q6w) or once every 8 weeks (q8w) to once every 12 weeks (q12w) based on a disease activity assessment at any scheduled treatment visit. In another embodiment, a dosing frequency or an administration interval of the VEGF antagonist A (e.g., brolucizumab) can be adjusted by increasing the dosing interval from once every 12 weeks (q12w) or to once every 18 weeks (q18w) or to once every 24 weeks (q24w) based on a disease activity assessment at any scheduled treatment visit.

In certain embodiments, the VEGF antagonist A used in the methods and the uses of the disclosure is an anti-VEGF antibody, in a particular wherein the anti-VEGF antibody is a single chain antibody (scFv) or Fab fragment, more particularly wherein the anti-VEGF antibody is brolucizumab. In certain embodiments, the methods and the uses of the disclosure comprise administering to the patient one or more doses of the VEGF antagonist A, wherein the VEGF antagonist A is brolucizumab and the dose of the VEGF antagonist A (e.g., the initial dose and the following doses) is about 3 mg to about 6 mg, in particular about 3 mg or about 6 mg, more particularly 6 mg.

In certain embodiments, the VEGF antagonist A is brolucizumab and the VEGF antagonist B is ranibizumab or aflibercept.

Non-limiting embodiments of the present disclosure are described in the following embodiments:

Embodiment 1: A method for treating ocular disease in a patient pretreated with one or more doses of a VEGF antagonist B, the method comprising administering to the patient an initial dose of a VEGF antagonist A followed by one or more additional doses of the VEGF antagonist A in an administration interval of no less than about 8 weeks, e.g., of no less than 8 weeks, and/or as individualized by a physician based on a disease activity assessment.

Embodiment 2: A method for treating ocular disease in a patient pretreated with one or more doses of a VEGF antagonist B, the method comprising administering to the patient an initial dose of a VEGF antagonist A followed by one or more additional doses of the VEGF antagonist A in an administration interval according to a maintenance regimen of the VEGF antagonist A for the treatment of the ocular disease.

Embodiment 3: The method of embodiment 1 or embodiment 2, wherein the method comprises discontinuing treatment with the VEGF antagonist B.

Embodiment 4: The method of embodiment 1 or 2, wherein the VEGF antagonist A is administered in replacement of the VEGF antagonist B and no additional or alternative VEGF antagonists are administered to the patient during the administration of the VEGF antagonist A.

Embodiment 5: The method of any one of the preceding embodiments, wherein the patient pretreated with one or more doses of the VEGF antagonist B has suboptimal anatomically controlled ocular disease.

Embodiment 6: The method of any one of the preceding embodiments, wherein presence of ocular disease activity was identified in the patient pretreated with one or more doses of the VEGF antagonist B.

Embodiment 7: The method of embodiment 6, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 8: The method of any one of embodiments 1 to 4, wherein the patient was intolerant to the treatment with the VEGF antagonist B.

Embodiment 9: The method of any one of the preceding embodiments, wherein the patient was pretreated with the VEGF antagonist B for at least 3 months or longer, preferably for at least 6 months or longer.

Embodiment 10: The method of any one of the preceding embodiments, wherein the VEGF antagonist B was administered to the patient in an administration interval, e.g., an injection interval, between about ≥4 and about ≤12 weeks, e.g., about ≥4 and about ≤10 weeks, in particular between about ≥6 and about ≤10 weeks, or about ≥8 and about ≤12 weeks.

Embodiment 11: The method of any one of the preceding embodiments, wherein the initial dose of the VEGF antagonist A is administered to the patient between about ≥4 and about ≤12 weeks, in particular between about ≥4 and about ≤10 weeks, more particularly between about ≥6 and about ≤10 weeks, after the last dose of the VEGF antagonist B was administered to the patient.

Embodiment 12: The method of any one of the preceding embodiments, wherein the initial dose of the VEGF antagonist A is followed by one or more doses of the VEGF antagonist A in an administration interval as individualized by a physician based on a disease activity assessment or in an administration interval between about ≥8 and about ≤24 weeks, e.g., between about ≥8 and about ≤12 weeks.

Embodiment 13: The method of embodiment 12, wherein the initial dose of the VEGF antagonist A is followed by administering to the patient one or more doses of the VEGF antagonist A once every 8 weeks (q8w regimen) or once every 12 weeks (q12w regimen) and/or as individualized by a physician based on a disease activity assessment.

Embodiment 14: The method of embodiment 13, further comprising assessing the patient for ocular disease activity before or after administering every q8w or q12w dose of the VEGF antagonist A.

Embodiment 15: The method of embodiment 1 or any one of embodiments 12 to 14, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

Embodiment 16: The method of embodiment 14 or 15, wherein if presence of ocular disease activity is identified after a q12w dose of the VEGF antagonist A, the patient is switched to a q8w regimen of the VEGF antagonist A.

Embodiment 17: The method of embodiment 16, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 18: The method of embodiment 17, wherein the presence of ocular disease activity includes one or more of the following:
(i) decrease in BCVA of ≥5 letters, in particular:
   decrease in BCVA of ≥5 letters at Week 8 or Week 12 after the last administration of the VEGF antagonist A compared to a baseline BCVA, wherein the baseline BCVA was assessed prior to the last administration of the VEGF antagonist A, or
   decrease in BCVA of ≥5 letters over 4 months or longer, e.g., over 6 months or longer, of the administration of the VEGF antagonist A compared to a baseline BCVA, wherein the baseline BCVA was assessed 4 months or longer, e.g., 6 months or longer, prior to the last administration of the VEGF antagonist A;
(ii) decrease in VA of ≥3 letters, in particular:
   decrease in VA of ≥3 letters at Week 8 or Week 12 after the last administration of the VEGF antagonist A compared to a baseline VA, wherein the baseline VA was assessed prior to the last administration of the VEGF antagonist A, or
   decrease in VA of ≥3 letters over 4 months or longer, e.g., over 6 months or longer, of the administration of the VEGF antagonist A compared to a baseline VA, wherein the baseline VA was assessed 4 months or longer, e.g., 6 months or longer, prior to the last administration of the VEGF antagonist A;
(iii) CSFT increase ≥50 μm, e.g., ≥75 μm, in particular:
   CSFT increase ≥50 μm, e.g., ≥75 μm, at Week 8 or Week 12 after the last administration of the VEGF antagonist A compared to a baseline CSFT, wherein the baseline CSFT was assessed prior to the last administration of the VEGF antagonist A, or
   CSFT increase ≥50 μm, e.g., ≥75 μm, over 4 months or longer, e.g., over 6 months or longer, of the administration of the VEGF antagonist A compared to a baseline CSFT, wherein the baseline CSFT was assessed 4 months or longer, e.g., 6 months or longer, prior to the last administration of the VEGF antagonist A;
(iv) new or persistent or recurrent intraretinal cysts (IRC) and/or intraretinal fluid (IRF) and/or subretinal fluid (SRF), in particular:
   new or persistent or recurrent intraretinal cysts (IRC) and/or intraretinal fluid (IRF) and/or subretinal fluid (SRF) at Week 8 or Week 12 after the last administration of the VEGF antagonist A compared to a baseline IRC and/or IRF and/or SRF, wherein the baseline IRC and/or IRF and/or SRF was assessed prior to the last administration of the VEGF antagonist A, or
   new or persistent or recurrent intraretinal cysts (IRC) and/or intraretinal fluid (IRF) and/or subretinal fluid (SRF) over 6 months or longer, of the administration of the VEGF antagonist A compared to a baseline IRC and/or IRF and/or SRF, wherein the baseline IRC and/or IRF and/or SRF was assessed 4 months or longer, e.g., 6 months or longer, prior to the last administration of the VEGF antagonist A.

Embodiment 19: The method of any of the preceding embodiments, wherein the ocular disease is selected from the list consisting of abnormal angiogenesis, choroidal neovascularization (CNV), retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

Embodiment 20: The method of embodiment 19, wherein the ocular disease is neovascular age-related macular degeneration (nAMD).

Embodiment 21: The method of embodiment 19, wherein the ocular disease is choroidal neovascularization (CNV) associated with nAMD.

Embodiment 22: The method of embodiment 19, wherein the ocular disease is Diabetic Macular Edema (DME).

Embodiment 23: The method of embodiment 19, wherein the ocular disease is diabetic retinopathy (DR).

Embodiment 24: The method of embodiment 19, wherein the ocular disease is Retinal Vein Occlusion (RVO).

Embodiment 25: The method of any one of preceding embodiments, wherein the patient is a human.

Embodiment 26: The method of any one of the preceding embodiments, wherein the VEGF antagonist A is different from the VEGF antagonist B.

Embodiment 27: The method of any one of preceding embodiments, wherein the VEGF antagonist A is an anti-VEGF antibody, e.g., a single chain antibody (scFv) or Fab fragment.

Embodiment 28: The method any one of preceding embodiments, wherein the VEGF antagonist A is an anti-VEGF antibody comprising the sequence of SEQ ID NO: 3.

Embodiment 29: The method of any one of preceding embodiments, wherein the anti-VEGF antagonist A is brolucizumab.

Embodiment 30: The method of any one of preceding embodiments wherein the VEGF antagonist A is administered by an injection, e.g., intravitreal injection.

Embodiment 31: The method of any one of preceding embodiments wherein the dose of the VEGF antagonist A is from about 3 mg to about 6 mg, in particular about 3 mg or about 6 mg, more particularly 6 mg.

Embodiment 32: The method of any one of preceding embodiments, wherein the VEGF antagonist B is an anti-VEGF antibody.

Embodiment 33: The method of any one of embodiments 1 to 31, wherein the VEGF antagonist B is selected from the group consisting of aflibercept, ranibizumab, faricimab, conbercept and abicipar.

Embodiment 34: The method of any one of preceding embodiments wherein the VEGF antagonist B is administered by an injection, e.g., intravitreal injection.

Embodiment 35: The method of embodiment 33, wherein the VEGF antagonist B is aflibercept and wherein the dose of the VEGF antagonist B is about 2 mg, in particular 2 mg.

Embodiment 36: The method of embodiment 33, wherein the VEGF antagonist B is ranibizumab and wherein the dose of the VEGF antagonist B is about 0.5 mg, in particular 0.5 mg.

Embodiment 37: A VEGF antagonist A for use as a medicament for treating ocular disease in a patient pretreated with one or more doses of a VEGF antagonist B, wherein the VEGF antagonist A is administered to the patient as an initial dose followed by one or more additional doses in an administration interval od no less than about 8 weeks, e.g., of no less than 8 weeks, and/or as individualized by a physician based on a disease activity assessment.

Embodiment 38: A VEGF antagonist A for use as a medicament for treating ocular disease in a patient pretreated with one or more doses of a VEGF antagonist B, wherein the VEGF antagonist A is administered to the patient as an initial dose followed by one or more additional doses in an administration interval according to a maintenance regimen of the VEGF antagonist A for the treatment of the ocular disease.

Embodiment 39: The VEGF antagonist A for use according to embodiment 37 or embodiment 38, wherein the use comprises discontinuing treatment with the VEGF antagonist B.

Embodiment 40: The VEGF antagonist A for use according to embodiment 37 or embodiment 38, wherein the VEGF antagonist A is administered in replacement of the VEGF antagonist B and no additional or alternative VEGF antagonists are administered to the patient during administration of the VEGF antagonist A.

Embodiment 41: The VEGF antagonist A for use according to any one of embodiments 37 to 40, wherein the patient pretreated with one or more doses of the VEGF antagonist B has suboptimal anatomically controlled ocular disease.

Embodiment 42: The VEGF antagonist A for use according to any one of embodiments 37 to 41, wherein presence of ocular disease activity was identified in the patient pretreated with one or more doses of the VEGF antagonist B.

Embodiment 43: The VEGF antagonist A for use according to embodiment 42, wherein the presence of ocular disease includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 44: The VEGF antagonist A for use according to any one of embodiments 37 to 41, wherein the patient was intolerant to the treatment with the VEGF antagonist B.

Embodiment 45: The VEGF antagonist A for use according to any one of embodiments 37 to 44, wherein the patient was pretreated with the VEGF antagonist B for at least 3 months or longer, preferably for at least 6 months or longer.

Embodiment 46: The VEGF antagonist A for use according to any one of embodiments 37 to 45, wherein the VEGF antagonist B was administered to the patient in an administration interval, e.g., an injection interval, between about ≥4 and about ≤12 weeks, e.g., about ≥4 and about ≤10 weeks, in particular between about ≥6 and about ≤10 weeks, or about ≥8 and about ≤12 weeks.

Embodiment 47: The VEGF antagonist A for use according to any one of embodiments 37 to 46, wherein the initial dose of the VEGF antagonist A is administered to the patient between about ≥4 and about ≤12 weeks, in particular between about ≥4 and about ≤10 weeks, more particularly between about ≥6 and about ≤10 weeks, after the last dose of the VEGF antagonist B was administered to the patient.

Embodiment 48: The VEGF antagonist A for use according to any one of embodiments 37 to 47, wherein the initial dose of the VEGF antagonist A is followed by one or more doses of the VEGF antagonist A in an administration interval as individualized by a physician based on a disease activity assessment or in an administration interval between about ≥8 and about ≤24 weeks, e.g., between about ≥8 and about ≤12 weeks.

Embodiment 49: The VEGF antagonist A for use according to embodiment 48, wherein the initial dose of the VEGF antagonist A is followed by administering to the patient one or more doses of the VEGF antagonist A once every 8 weeks (q8w regimen) or once every 12 weeks (q12w regimen) and/or as individualized by a physician based on a disease activity assessment.

Embodiment 50: The VEGF antagonist A for use according to embodiment 49, further comprising assessing the patient for ocular disease activity before or after administering every q8w or q12w dose of the VEGF antagonist A.

Embodiment 51: The VEGF antagonist A for use according to embodiment 37 or according to any one of embodiments 48 to 50, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

Embodiment 52: The VEGF antagonist A for use according to embodiment 50 or 51, wherein if presence of ocular disease activity is identified after a q12w dose of the VEGF antagonist A, the patient is switched to a q8w regimen of the VEGF antagonist A.

Embodiment 53: The VEGF antagonist A for use according to embodiment 52, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 54: The VEGF antagonist A for use according to embodiment 53, wherein the presence of ocular disease activity includes one or more of the following:
(i) decrease in BCVA of ≥5 letters, in particular:
   decrease in BCVA of ≥5 letters at Week 8 or Week 12 after the last administration of the VEGF antagonist A compared to a baseline BCVA, wherein the baseline BCVA was assessed prior to the last administration of the VEGF antagonist A, or
   decrease in BCVA of ≥5 letters over 4 months or longer, e.g., over 6 months or longer, of the administration of the VEGF antagonist A compared to a baseline BCVA, wherein the baseline BCVA was assessed 4 months or longer, e.g., 6 months or longer, prior to the last administration of the VEGF antagonist A;
(ii) decrease in VA of ≥3 letters, in particular:
   decrease in VA of ≥3 letters at Week 8 or Week 12 after the last administration of the VEGF antagonist A compared to a baseline VA, wherein the baseline VA was assessed prior to the last administration of the VEGF antagonist A, or
   decrease in VA of ≥3 letters over 4 months or longer, e.g., over 6 months or longer, of the administration of the VEGF antagonist A compared to a baseline VA, wherein the baseline VA was assessed 4 months or longer, e.g., 6 months or longer, prior to the last administration of the VEGF antagonist A;
(iii) CSFT increase ≥50 μm, e.g., ≥75 μm, in particular:
   CSFT increase ≥50 μm, e.g., ≥75 μm, at Week 8 or Week 12 after the last administration of the VEGF antagonist A compared to a baseline CSFT, wherein the baseline CSFT was assessed prior to the last administration of the VEGF antagonist A, or
   CSFT increase ≥50 μm, e.g., ≥75 μm, over 4 months or longer, e.g., over 6 months or longer, of the administration of the VEGF antagonist A compared to a baseline CSFT, wherein the baseline CSFT was assessed 4 months or longer, e.g., 6 months or longer, prior to the last administration of the VEGF antagonist A;
(iv) new or persistent or recurrent intraretinal cysts (IRC) and/or intraretinal fluid (IRF) and/or subretinal fluid (SRF), in particular:
   new or persistent or recurrent intraretinal cysts (IRC) and/or intraretinal fluid (IRF) and/or subretinal fluid (SRF) at Week 8 or Week 12 after the last administration of the VEGF antagonist A compared to a baseline IRC and/or IRF and/or SRF, wherein the baseline IRC and/or IRF and/or SRF was assessed prior to the last administration of the VEGF antagonist A, or
   new or persistent or recurrent intraretinal cysts (IRC) and/or intraretinal fluid (IRF) and/or subretinal fluid (SRF) over 6 months or longer, of the administration of the VEGF antagonist A compared to a baseline IRC and/or IRF and/or SRF, wherein the baseline IRC and/or IRF and/or SRF was assessed 4 months or longer, e.g., 6 months or longer, prior to the last administration of the VEGF antagonist A.

Embodiment 55: The VEGF antagonist A for use according to any of embodiments 37 to 54, wherein the ocular disease is selected from the list consisting of abnormal angiogenesis, choroidal neovascularization (CNV), retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

Embodiment 56: The VEGF antagonist A for use according to embodiment 55, wherein the ocular disease is neovascular age-related macular degeneration (nAMD).

Embodiment 57: The VEGF antagonist A for use according to embodiment 55, wherein the ocular disease is choroidal neovascularization (CNV) associated with nAMD.

Embodiment 58: The VEGF antagonist A for use according to embodiment 55, wherein the ocular disease is Diabetic Macular Edema (DME).

Embodiment 59: The VEGF antagonist A for use according to embodiment 55, wherein the ocular disease is diabetic retinopathy (DR).

Embodiment 60: The VEGF antagonist A for use according to embodiment 55, wherein the ocular disease is Retinal Vein Occlusion (RVO).

Embodiment 61: The VEGF antagonist A for use according to any one of embodiments 37 to 60, wherein the patient is a human.

Embodiment 62: The VEGF antagonist A for use according to any one of embodiments 37 to 61, wherein the VEGF antagonist A is different from the VEGF antagonist B.

Embodiment 63: The VEGF antagonist A for use according to any one of embodiments 37 to 62, wherein the VEGF antagonist A is an anti-VEGF antibody, e.g., a single chain antibody (scFv) or Fab fragment.

Embodiment 64: The VEGF antagonist A for use according to any one of embodiments 37 to 63, wherein the VEGF antagonist A is an anti-VEGF antibody comprising the sequence of SEQ ID NO: 3.

Embodiment 65: The VEGF antagonist A for use according to any one of embodiments 37 to 64, wherein the anti-VEGF antagonist A is brolucizumab.

Embodiment 66: The VEGF antagonist A for use according to any one of embodiments 37 to 65, wherein the VEGF antagonist A is administered by an injection, e.g., intravitreal injection.

Embodiment 67: The VEGF antagonist A for use according to any one of embodiments 37 to 66, wherein the dose of the VEGF antagonist A is from about 3 mg to about 6 mg, in particular about 3 mg or about 6 mg, more particularly 6 mg.

Embodiment 68: The VEGF antagonist A for use according to any one of embodiments 37 to 67, wherein the VEGF antagonist B is an anti-VEGF antibody.

Embodiment 69: The VEGF antagonist A for use according to any one of embodiments 37 to 67, wherein the VEGF antagonist B is selected from the group consisting of aflibercept, ranibizumab, faricimab, conbercept and abicipar.

Embodiment 70: The VEGF antagonist A for use according to any one of embodiments 37 to 69, wherein the VEGF antagonist B is administered by an injection, e.g., intravitreal injection.

Embodiment 71: The VEGF antagonist A for use according to embodiment 70, wherein the VEGF antagonist B is aflibercept and wherein the dose of the VEGF antagonist B is about 2 mg, in particular 2 mg.

Embodiment 72: The VEGF antagonist A for use according to embodiment 70, wherein the VEGF antagonist B is ranibizumab and wherein the dose of the VEGF antagonist B is about 0.5 mg, in particular 0.5 mg.

Embodiment 73: A pharmaceutical composition comprising a VEGF antagonist A for use as a medicament for treating ocular disease in a patient pretreated with one or more doses of a VEGF antagonist B, wherein the pharmaceutical composition comprising a VEGF antagonist A is administered to the patient as an initial dose followed by one or more additional doses in an administration interval of no less than about 8 weeks, e.g., of no less than 8 weeks, and/or as individualized by a physician based on a disease activity assessment.

Embodiment 74: A pharmaceutical composition comprising a VEGF antagonist A for use as a medicament for treating ocular disease in a patient pretreated with one or more doses of a VEGF antagonist B, wherein the pharmaceutical composition comprising a VEGF antagonist A is administered to the patient as an initial dose followed by one or more additional doses in an administration interval according to a maintenance regimen of the VEGF antagonist A for the treatment of the ocular disease.

Embodiment 75: Use of a VEGF antagonist A for the manufacture of a medicament for treating ocular disease in a patient pretreated with one or more doses of a VEGF antagonist B, wherein the use comprises administering to the patient an initial dose of the VEGF antagonist A followed by one or more additional doses of the VEGF antagonist A in an administration interval of no less than about 8 weeks, e.g., of no less than 8 weeks, and/or as individualized by a physician based on a disease activity assessment.

Embodiment 76: Use of a VEGF antagonist A for the manufacture of a medicament for treating ocular disease in a patient pretreated with one or more doses of a VEGF antagonist B, wherein the use comprises administering to the patient an initial dose of the VEGF antagonist A followed by one or more additional doses of the VEGF antagonist A in an administration interval according to a maintenance regimen of the VEGF antagonist A for the treatment of the ocular disease.

Embodiment 77: Use of a VEGF antagonist A for treating ocular disease in a patient pretreated with one or more doses of a VEGF antagonist B, wherein the use comprises administering to the patient an initial dose of the VEGF antagonist A followed by one or more additional doses of the VEGF antagonist A in an administration interval of no less than about 8 weeks, e.g., of no less than 8 weeks, and/or as individualized by a physician based on a disease activity assessment.

Embodiment 78: Use of a VEGF antagonist A for treating ocular disease in a patient pretreated with one or more doses of a VEGF antagonist B, wherein the use comprises administering to the patient an initial dose of the VEGF antagonist A followed by one or more additional doses of the VEGF antagonist A in an administration interval according to a maintenance regimen of the VEGF antagonist A for the treatment of the ocular disease.

Embodiment 79: The VEGF antagonist A for use according to any one of embodiments 37 to 72 or the pharmaceutical composition for use of embodiment 73 or 74 or the use of any one of embodiments 75 to 78, wherein the use comprises discontinuing treatment with the VEGF antagonist B.

Embodiment 80: The VEGF antagonist A for use according to any one of embodiments 37 to 72 or the pharmaceutical composition for use of embodiment 73 or 74 or the use of any one of embodiments 75 to 78, wherein the VEGF antagonist A is administered in replacement of the VEGF antagonist B and no additional or alternative VEGF antagonists are administered to the patient during administration of the VEGF antagonist A.

Embodiment 81: A method for treating ocular disease in a patient, the method comprising administering to the patient an initial dose of a VEGF antagonist followed by a second dose at least 8 weeks after the initial dose, e.g., 8, 9, 10, 11, 12, 13, 14, 15, 16, 17, 18, 19, 20, 21, 22, 23, or 24 weeks.

Embodiment 82: The method of embodiment 81, further comprising administering one or more additional doses of the VEGF antagonist after the second dose, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks.

Embodiment 83: A method for treating ocular disease in a patient, the method comprising administering to the patient an initial dose of a VEGF antagonist followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

Embodiment 84: The method of any one of embodiments 81 to 83, wherein the method does not comprise administering to the patient one or more additional doses of the VEGF antagonist in an administration interval of less than 8 weeks.

Embodiment 85: A method for treating ocular disease in a patient, the method comprising administering to the patient one initial dose of a VEGF antagonist followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

Embodiment 86: The method of embodiment 85, wherein the maintenance regimen of the VEGF antagonist consists of 2, 3, 4, 5, 6 or more doses administered in an administration interval of at least 8 weeks, e.g., 8, 9, 10, 11, 12, 13, 14, 15, 16, 17, 18, 19, 20, 21, 22, 23, or 24 week intervals.

Embodiment 87: The method of embodiment 85 or 86, wherein the method does not comprise administering to the patient one or more additional doses of the VEGF antagonist in an administration interval according to a loading regimen of the VEGF antagonist for the treatment of the ocular disease.

Embodiment 88: The method of embodiment 87, wherein the loading regimen of the VEGF antagonist consists of 2, 3, 4, 5, or 6 doses of the VEGF antagonist administered at q4w or q6w intervals.

Embodiment 89: The method according to any one of embodiments 81 to 88, wherein the initial dose of the VEGF antagonist is followed by one or more doses of the VEGF antagonist in an administration interval as individualized by a physician based on a disease activity assessment and/or in an administration interval between ≥8 and ≤24 weeks, e.g., between ≥8 and ≤12 weeks.

Embodiment 90: The method of embodiment 89, wherein the initial dose of the VEGF antagonist is followed by administering to the patient one or more doses of the VEGF antagonist once every 8 weeks (q8w regimen) or once every 12 weeks (q12w regimen) and/or as individualized by a physician based on a disease activity assessment.

Embodiment 91: The method of embodiment 90, further comprising assessing the patient for ocular disease activity before or after administering every q8w or q12w dose of the VEGF antagonist.

Embodiment 92: The method according to any one of embodiments 81 to 91, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

Embodiment 93: The method of embodiment 91 or 92, wherein if presence of ocular disease activity is identified after a q12w dose of the VEGF antagonist, the patient is switched to a q8w regimen of the VEGF antagonist.

Embodiment 94: The method of embodiment 93, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 95: The method of embodiment 94, wherein the presence of ocular disease activity includes one or more of the following:
(i) decrease in BCVA of ≥5 letters, e.g.,:
   decrease in BCVA of ≥5 letters at Week 8 or Week 12 after the last administration of the VEGF antagonist compared to a baseline BCVA, wherein the baseline BCVA was assessed prior to the last administration of the VEGF antagonist, or
   decrease in BCVA of ≥5 letters over 4 months or longer, e.g., over 6 months or longer, of the administration of the VEGF antagonist compared to a baseline BCVA, wherein the baseline BCVA was assessed 4 months or longer, e.g., 6 months or longer, prior to the last administration of the VEGF antagonist;
(ii) decrease in VA of ≥3 letters, e.g.,:
   decrease in VA of ≥3 letters at Week 8 or Week 12 after the last administration of the VEGF antagonist compared to a baseline VA, wherein the baseline VA was assessed prior to the last administration of the VEGF antagonist, or
   decrease in VA of ≥3 letters over 4 months or longer, e.g., over 6 months or longer, of the administration of the VEGF antagonist compared to a baseline VA, wherein the baseline VA was assessed 4 months or longer, e.g., 6 months or longer, prior to the last administration of the VEGF antagonist;
(iii) CSFT increase ≥50 μm, e.g., ≥75 μm, e.g.,:
   CSFT increase ≥50 μm, e.g., ≥75 μm, at Week 8 or Week 12 after the last administration of the VEGF antagonist compared to a baseline CSFT, wherein the baseline CSFT was assessed prior to the last administration of the VEGF antagonist, or
   CSFT increase ≥50 μm, e.g., ≥75 μm, over 4 months or longer, e.g., over 6 months or longer, of the administration of the VEGF antagonist compared to a baseline CSFT, wherein the baseline CSFT was assessed 4 months or longer, e.g., 6 months or longer, prior to the last administration of the VEGF antagonist;
(iv) new or persistent or recurrent intraretinal cysts (IRC) and/or intraretinal fluid (IRF) and/or subretinal fluid (SRF), e.g.,:
   new or persistent or recurrent intraretinal cysts (IRC) and/or intraretinal fluid (IRF) and/or subretinal fluid (SRF) at Week 8 or Week 12 after the last administration of the VEGF antagonist compared to a baseline IRC and/or IRF and/or SRF, wherein the baseline IRC and/or IRF and/or SRF was assessed prior to the last administration of the VEGF antagonist, or
   new or persistent or recurrent intraretinal cysts (IRC) and/or intraretinal fluid (IRF) and/or subretinal fluid (SRF) over 6 months or longer, of the administration of the VEGF antagonist compared to a baseline IRC and/or IRF and/or SRF, wherein the baseline IRC and/or IRF and/or SRF was assessed 4 months or longer, e.g., 6 months or longer, prior to the last administration of the VEGF antagonist.

Embodiment 96: The method according to any one of embodiments 81 to 95, wherein the ocular disease is selected from the list consisting of abnormal angiogenesis, choroidal neovascularization (CNV), retinal vascular permeability, retinal edema, diabetic retinopathy (e.g., proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

Embodiment 97: The method of embodiment 96, wherein the ocular disease is neovascular age-related macular degeneration (nAMD).

Embodiment 98: The method of embodiment 96, wherein the ocular disease is Diabetic Macular Edema (DME).

Embodiment 99: The method according to any one of embodiments 81 to 98, wherein the VEGF antagonist is an anti-VEGF antibody, e.g., a single chain antibody (scFv) or Fab fragment.

Embodiment 100: The method according to any one of embodiments 81 to 99, wherein the VEGF antagonist is an anti-VEGF antibody comprising the sequence of SEQ ID NO: 3.

Embodiment 101: The method according to any one of embodiments 81 to 100, wherein the anti-VEGF antagonist is brolucizumab.

Embodiment 102: The method according to any one of embodiments 81 to 101, wherein the VEGF antagonist is administered by an injection, e.g., intravitreal injection.

Embodiment 103: The method according to any one of embodiments 81 to 102, wherein the dose of the VEGF antagonist is from about 3 mg to about 6 mg, e.g., about 3 mg or about 6 mg, e.g., 6 mg.

Embodiment 104: The method according to any one of embodiments 81 to 103, wherein the patient is a human.

Embodiment 105: The method according to any one of embodiments 81 to 104, wherein the patient is a naïve patient.

Embodiment 106: The method according to any one of embodiments 81 to 104, wherein the patient is a pretreated patient, e.g., a patient pretreated with one or more doses of a VEGF antagonist different from the VEGF antagonist of any one of embodiments 81 to 103.

Embodiment 107: The method of embodiment 106, wherein the patient was pretreated with a VEGF antagonist selected from the group consisting of aflibercept, ranibizumab, faricimab, conbercept and abicipar.

Embodiment 108: A VEGF antagonist for use as a medicament for treating ocular disease in a patient, wherein the VEGF antagonist is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

Embodiment 109: The VEGF antagonist for use of embodiment 108, wherein one or more additional doses of the VEGF antagonist are administered after the second dose, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks.

Embodiment 110: A VEGF antagonist for use as a medicament for treating ocular disease in a patient, wherein the VEGF antagonist is administered to the patient as an initial dose followed by one or more additional doses, wherein the one or more additional doses of the VEGF antagonist are administered at least 8 weeks after the initial dose and each of the one or more additional doses after the initial dose are administered in an administration interval of at least 8 weeks.

Embodiment 111: A VEGF antagonist for use as a medicament for treating ocular disease in a patient, wherein the VEGF antagonist is administered to the patient as one initial dose followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

Embodiment 112: The VEGF antagonist for use of embodiment 111, wherein the maintenance regimen of the VEGF antagonist consists of 2, 3, 4, 5, 6 or more doses administered in an administration interval of at least 8 weeks, e.g., 8, 9, 10, 11, 12, 13, 14, 15, 16, 17, 18, 19, 20, 21, 22, 23, or 24 week intervals.

Embodiment 113: The VEGF antagonist for use of any one of embodiment 108 to 112, wherein the initial dose of the VEGF antagonist is followed by one or more doses of the VEGF antagonist in an administration interval as individualized by a physician based on a disease activity assessment and/or in an administration interval between ≥8 and ≤24 weeks, e.g., between ≥8 and ≤12 weeks.

Embodiment 114: The VEGF antagonist for use of embodiment 113, wherein the initial dose of the VEGF antagonist is followed by administering to the patient one or more doses of the VEGF antagonist once every 8 weeks (q8w regimen) or once every 12 weeks (q12w regimen) and/or as individualized by a physician based on a disease activity assessment.

Embodiment 115: The VEGF antagonist for use of embodiment 114, further comprising assessing the patient for ocular disease activity before or after administering every q8w or q12w dose of the VEGF antagonist.

Embodiment 116: The VEGF antagonist for use of any one of embodiments 113 to 115, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

Embodiment 117: The VEGF antagonist for use of embodiment 113 or 116, wherein if presence of ocular disease activity is identified after a q12w dose of the VEGF antagonist, the patient is switched to a q8w regimen of the VEGF antagonist.

Embodiment 118: The VEGF antagonist for use of embodiment 117 wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

Embodiment 119: The VEGF antagonist for use of embodiment 118, wherein the presence of ocular disease activity includes one or more of the following:
(i) decrease in BCVA of ≥5 letters, e.g.,:
   decrease in BCVA of ≥5 letters at Week 8 or Week 12 after the last administration of the VEGF antagonist compared to a baseline BCVA, wherein the baseline BCVA was assessed prior to the last administration of the VEGF antagonist, or
   decrease in BCVA of ≥5 letters over 4 months or longer, e.g., over 6 months or longer, of the administration of the VEGF antagonist compared to a baseline BCVA, wherein the baseline BCVA was assessed 4 months or longer, e.g., 6 months or longer, prior to the last administration of the VEGF antagonist;
(ii) decrease in VA of ≥3 letters, e.g.,:
   decrease in VA of ≥3 letters at Week 8 or Week 12 after the last administration of the VEGF antagonist compared to a baseline VA, wherein the baseline VA was assessed prior to the last administration of the VEGF antagonist, or
   decrease in VA of ≥3 letters over 4 months or longer, e.g., over 6 months or longer, of the administration of the VEGF antagonist compared to a baseline VA, wherein the baseline VA was assessed 4 months or longer, e.g., 6 months or longer, prior to the last administration of the VEGF antagonist;
(iii) CSFT increase ≥50 μm, e.g., ≥75 μm, e.g.,:
   CSFT increase ≥50 μm, e.g., ≥75 μm, at Week 8 or Week 12 after the last administration of the VEGF antagonist compared to a baseline CSFT, wherein the baseline CSFT was assessed prior to the last administration of the VEGF antagonist, or
   CSFT increase ≥50 μm, e.g., ≥75 μm, over 4 months or longer, e.g., over 6 months or longer, of the administration of the VEGF antagonist compared to a baseline CSFT, wherein the baseline CSFT was assessed 4 months or longer, e.g., 6 months or longer, prior to the last administration of the VEGF antagonist;
(iv) new or persistent or recurrent intraretinal cysts (IRC) and/or intraretinal fluid (IRF) and/or subretinal fluid (SRF), e.g.,:
   new or persistent or recurrent intraretinal cysts (IRC) and/or intraretinal fluid (IRF) and/or subretinal fluid (SRF) at Week 8 or Week 12 after the last administration of the VEGF antagonist compared to a baseline IRC and/or IRF and/or SRF, wherein the baseline IRC and/or IRF and/or SRF was assessed prior to the last administration of the VEGF antagonist, or
   new or persistent or recurrent intraretinal cysts (IRC) and/or intraretinal fluid (IRF) and/or subretinal fluid (SRF) over 6 months or longer, of the administration of the VEGF antagonist compared to a baseline IRC and/or IRF and/or SRF, wherein the baseline IRC and/or IRF and/or SRF was assessed 4 months or longer, e.g., 6 months or longer, prior to the last administration of the VEGF antagonist.

Embodiment 120: The VEGF antagonist for use of any one of embodiments 108 to 119, wherein the ocular disease is selected from the list consisting of abnormal angiogenesis, choroidal neovascularization (CNV), retinal vascular permeability, retinal edema, diabetic retinopathy (e.g., proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

Embodiment 121: The VEGF antagonist for use of embodiment 120, wherein the ocular disease is neovascular age-related macular degeneration (nAMD).

Embodiment 122: The VEGF antagonist for use of embodiment 120, wherein the ocular disease is Diabetic Macular Edema (DME).

Embodiment 123: The VEGF antagonist for use of any one of embodiments 108 to 122, wherein the VEGF antagonist is an anti-VEGF antibody, e.g., a single chain antibody (scFv) or Fab fragment.

Embodiment 124: The VEGF antagonist for use of any one of embodiments 108 to 123, wherein the VEGF antagonist is an anti-VEGF antibody comprising the sequence of SEQ ID NO: 3.

Embodiment 125: The VEGF antagonist for use of any one of embodiments 108 to 124, wherein the anti-VEGF antagonist is brolucizumab.

Embodiment 126: The VEGF antagonist for use of any one of embodiments 108 to 125, wherein the VEGF antagonist is administered by an injection, e.g., intravitreal injection.

Embodiment 127: The VEGF antagonist for use of any one of embodiments 108 to 126, wherein the dose of the VEGF antagonist is from about 3 mg to about 6 mg, e.g., about 3 mg or about 6 mg, e.g., 6 mg.

Embodiment 128: The VEGF antagonist for use of any one of embodiments 108 to 127, wherein the patient is a human.

Embodiment 129: The VEGF antagonist for use of any one of embodiments 108 to 128, wherein the patient is a naïve patient.

Embodiment 130: The VEGF antagonist for use of any one of embodiments 108 to 128, wherein the patient is a pretreated patient, e.g., a patient pretreated with one or more doses of a VEGF antagonist different from the VEGF antagonist of any one of embodiments 108 to 128.

Embodiment 131: The VEGF antagonist for use of embodiment 130, wherein the patient was pretreated with a VEGF antagonist selected from the group consisting of aflibercept, ranibizumab, faricimab, conbercept and abicipar.

Embodiment 132: A pharmaceutical composition comprising a VEGF antagonist for use as a medicament for treating ocular disease in a patient, wherein the pharmaceutical composition is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

Embodiment 133: The pharmaceutical composition for use of embodiment 132, wherein one or more additional doses of the pharmaceutical composition are administered after the second dose, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks.

Embodiment 134: A pharmaceutical composition comprising a VEGF antagonist for use as a medicament for treating ocular disease in a patient, wherein the pharmaceutical composition is administered to the patient as an initial dose followed by one or more additional doses, wherein the one or more additional doses of the pharmaceutical composition are administered at least 8 weeks after the initial dose and each of the one or more additional doses after the initial dose are administered in an administration interval of at least 8 weeks.

Embodiment 135: A pharmaceutical composition comprising a VEGF antagonist for use as a medicament for treating ocular disease in a patient, wherein the pharmaceutical composition is administered to the patient as one initial dose followed by a maintenance regimen of additional doses of the pharmaceutical composition administered in an administration interval of at least 8 weeks, e.g., 8, 9, 10, 11, 12, 13, 14, 15, 16, 17, 18, 19, 20, 21, 22, 23, or 24 week intervals.

Embodiment 136: The pharmaceutical composition for use of embodiment 135, wherein the maintenance regimen of the pharmaceutical composition consists of 2, 3, 4, 5, 6 or more doses administered in an administration interval of at least 8 weeks, e.g., 8, 9, 10, 11, 12, 13, 14, 15, 16, 17, 18, 19, 20, 21, 22, 23, or 24 week intervals.

Embodiment 137: The pharmaceutical composition for use of any one of embodiment 132 to 136, wherein the initial dose of the pharmaceutical composition is followed by one or more doses of the pharmaceutical composition in an administration interval as individualized by a physician based on a disease activity assessment and/or in an administration interval between ≥8 and ≤24 weeks, e.g., between ≥8 and ≤12 weeks.

Embodiment 138: Use of a VEGF antagonist for the manufacture of a medicament for treating ocular disease in a patient, the use comprising administering to the patient an initial dose of the VEGF antagonist followed by a second dose at least 8 weeks after the initial dose, e.g., 8, 9, 10, 11, 12, 13, 14, 15, 16, 17, 18, 19, 20, 21, 22, 23, or 24 weeks.

Embodiment 139: The use of embodiment 138, further comprising administering one or more additional doses of the VEGF antagonist after the second dose, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks.

Embodiment 140: Use of a VEGF antagonist for the manufacture of a medicament for treating ocular disease in a patient, the use comprising administering to the patient an initial dose of the VEGF antagonist followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

Embodiment 141: The use of any one of embodiments 138 to 140, wherein the use does not comprise administering to the patient one or more additional doses of the VEGF antagonist in an administration interval of less than 8 weeks.

Embodiment 142: Use of a VEGF antagonist for the manufacture of a medicament for treating ocular disease in a patient, the use comprising administering to the patient one initial dose of the VEGF antagonist followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

Embodiment 143: The use of embodiment 142, wherein the maintenance regimen of the VEGF antagonist consists of 2, 3, 4, 5, 6 or more doses administered in an administration interval of at least 8 weeks, e.g., 8, 9, 10, 11, 12, 13, 14, 15, 16, 17, 18, 19, 20, 21, 22, 23, or 24 week intervals.

Embodiment 144: The use of embodiment 142 or 143, wherein the use does not comprise administering to the patient one or more additional doses of the VEGF antagonist in an administration interval according to a loading regimen of the VEGF antagonist for the treatment of the ocular disease.

Embodiment 145: The use of embodiment 144, wherein the loading regimen of the VEGF antagonist consists of 2, 3, 4, 5, or 6 doses of the VEGF antagonist administered at q4w or q6w intervals.

Embodiment 146: The use of any one of embodiment 138 to 145, wherein the initial dose of the VEGF antagonist is followed by one or more doses of the VEGF antagonist in an administration interval as individualized by a physician based on a disease activity assessment and/or in an administration interval between ≥8 and ≤24 weeks, e.g., between ≥8 and ≤12 weeks.

Specific preferred embodiments of the invention will become evident from the following more detailed description of certain preferred embodiments and the claims.

### BRIEF DESCRIPTION OF THE DRAWINGS

FIG. 1. Graphs of model simulations of the drug concentration over time (PK) within the vitreous compartment of the eye and the level of free VEGF in the retina compartment of the eye. FIGS. 1A and 1C: ranibizumab PK in the vitreous and reduction in free VEGF in the retina following IVT administration of 0.5 mg drug every 4 weeks (Q4W). FIGS. 1B and 1D: aflibercept PK in the vitreous and reduction in free VEGF in the retina following IVT administration of 2.0 mg drug for a loading period consisting of one dose every 4 weeks (Q4W) for the first three months and a maintenance period consisting of one dose every 8 weeks (Q8W).

FIG. 2. Graphs of model simulations of brolucizumab PK in the vitreous compartment of the eye and the level of free VEGF in the retina compartment of the eye. FIGS. 2A and 2C: brolucizumab PK in the vitreous and reduction in free VEGF in the retina following IVT administration of 6.0 mg drug for a loading period consisting of one dose every 4 weeks (Q4W) for the first three months and a maintenance period consisting of one dose every 8 weeks (Q8W). FIGS. 2B and 2D: brolucizumab PK in the vitreous and reduction in free VEGF in the retina following IVT administration of 6.0 mg drug for a loading period consisting of one dose every 4 weeks (Q4W) for the first three months and a maintenance period consisting of one dose every twelve weeks (Q12W).

FIG. 3. Graphs of model simulations in which one VEGF antagonist is administered for a period of approximately one year and a second VEGF antagonist is administered for a subsequent period of approximately one year. Ranibizumab was administered by IVT at a dose of 0.5 mg every 4 weeks (Q4W) between Weeks 0 and 48. Brolucizumab was administered by IVT at a dose of 6.0 mg with a regimen consisting of a loading period (one dose every 4 weeks for the first three months) and a maintenance period (one dose every 8 weeks) between Weeks 52 and 100. FIG. 3A: drug PK in the vitreous for ranibizumab (dashed line) and brolucizumab (solid line). FIG. 3B: Free VEGF in the retina.

FIG. 4. Graphs of model simulations performed as described in FIG. 3 but without the loading doses of brolucizumab. FIG. 4 shows simulation results in which the administration of brolucizumab (following approximately one year of treatment with ranibizumab) omits the loading dose period and instead consists of one dose every 8 weeks starting at Week 52. The last administration of brolucizumab is on Week 100. FIG. 4A: drug PK in the vitreous for ranibizumab (dashed line) and brolucizumab (solid line). FIG. 4B: Free VEGF in the retina.

FIG. 5. Graphs of model simulations performed as described in FIG. 3 but with a brolucizumab dosing interval of Q12W during the maintenance period. FIG. 5A: drug PK in the vitreous for ranibizumab (dashed line) and brolucizumab (solid line). FIG. 5B: Free VEGF in the retina.

FIG. 6. Graphs of model simulations performed as described in FIG. 5 but without the loading doses of brolucizumab. FIG. 6 shows simulation results in which the administration of brolucizumab (following approximately one year of treatment with ranibizumab) omits the loading dose period and instead consists of one dose every 12 weeks starting at Week 52. The last administration of brolucizumab is on Week 100. FIG. 6A: drug PK in the vitreous for ranibizumab (dashed line) and brolucizumab (solid line). FIG. 6B: Free VEGF in the retina.

FIG. 7. Graphs of model simulations performed to assess the duration of time that free VEGF in the retina is maintained below indicated threshold concentrations of 5 pM and 10 pM following the last dose of ranibizumab. Ranibizumab was administered by IVT at a dose of 0.5 mg every 4 weeks (Q4W) between Weeks 0 and 48. The timing and degree of the recovery for free VEGF in the retina is shown to vary based upon the level of VEGF production in the eye (exemplified as Low, Mean, or High levels of VEGF production).

FIG. 8. Graphs of model simulations performed as described in FIG. 4 but with the first dose of brolucizumab at Day 49 following the last dose of ranibizumab and only under the condition of high VEGF production. This graph is intended to illustrate drug switching to brolucizumab under the simulation conditions in which free VEGF in the retina is first observed to surpass the indicated 5 pM threshold following the last dose of ranibizumab (as shown in FIG. 7). FIG. 8A: drug PK in the vitreous for ranibizumab (dashed line) and brolucizumab (solid line). FIG. 8B: Free VEGF in the retina.

FIG. 9. Graphs of model simulations in which one VEGF antagonist is administered for a period of approximately one year and a second VEGF antagonist is administered for a subsequent period of approximately one year. Aflibercept was administered by IVT at a dose of 2.0 mg with a regimen consisting of a loading period (one dose every 4 weeks for the first three months) and a maintenance period (one dose every 8 weeks) between Weeks 0 and 48. Brolucizumab was administered by IVT at a dose of 6.0 mg with a regimen consisting of a loading period (one dose every 4 weeks for the first three months) and a maintenance period (one dose every 8 weeks) between Weeks 56 and 96. FIG. 9A: drug PK in the vitreous for aflibercept (dashed line) and brolucizumab (solid line). FIG. 9B: Free VEGF in the retina.

FIG. 10. Graphs of model simulations performed as described in FIG. 9 but without the loading doses of brolucizumab. FIG. 9 shows simulation results in which the administration of brolucizumab (following approximately one year of treatment with aflibercept) omits the loading dose period and instead consists of one dose every 8 weeks starting at Week 56. FIG. 10A: drug PK in the vitreous for aflibercept (dashed line) and brolucizumab (solid line). FIG. 10B: Free VEGF in the retina.

FIG. 11. Graphs of model simulations performed as described in FIG. 9 but with a brolucizumab dosing interval of Q12W during the maintenance period. The last administration of brolucizumab is on Week 100. FIG. 11A: drug PK in the vitreous for aflibercept (dashed line) and brolucizumab (solid line). FIG. 11B: Free VEGF in the retina.

FIG. 12. Graphs of model simulations performed as described in FIG. 11 but without the loading doses of brolucizumab. FIG. 12 shows simulation results in which the administration of brolucizumab (following approximately one year of treatment with aflibercept) omits the loading dose period and instead consists of one dose every 12 weeks starting at Week 52. The last administration of brolucizumab is on Week 100. FIG. 12A: drug PK in the vitreous for aflibercept (dashed line) and brolucizumab (solid line). FIG. 12B: Free VEGF in the retina.

FIG. 13. Graphs of model simulations performed to assess the duration of time that free VEGF in the retina is maintained below indicated threshold concentrations of 5 pM and 10 pM following the last dose of aflibercept. Aflibercept was administered by IVT at a dose of 2.0 mg with a regimen consisting of a loading period (one dose every 4 weeks for the first three months) and a maintenance period (one dose every 8 weeks) between Weeks 0 and 48. The timing and degree of the recovery for free VEGF in the retina is shown to vary based upon the level of VEGF production in the eye (exemplified as Low, Mean, or High levels of VEGF production).

FIG. 14. Graphs of model simulations performed as described in FIG. 10 but with the first dose of brolucizumab at Day 87 following the last dose of aflibercept and only under the condition of high VEGF production. This graph is intended to illustrate drug switching to brolucizumab under the simulation conditions in which free VEGF in the retina is first observed to surpass the indicated 5 pM threshold following the last dose of aflibercept (as shown in FIG. 13). FIG. 14A: drug PK in the vitreous for aflibercept (dashed line) and brolucizumab (solid line). FIG. 14B: Free VEGF in the retina.

FIG. 15. Graphs of model simulations in which one VEGF antagonist is administered for a period of approximately one year and a second VEGF antagonist is administered for a subsequent period of approximately one year. Brolucizumab was administered by IVT at a dose of 6.0 mg with a regimen consisting of a loading period (one dose every 4 weeks for the first three months) and a maintenance period (one dose every 8 weeks) between Weeks 0 and 48. Aflibercept was administered by IVT at a dose of 2.0 mg with a regimen consisting of a loading period (one dose every 4 weeks for the first three months) and a maintenance period (one dose every 8 weeks) between Weeks 56 and 96. FIG. 15A: drug PK in the vitreous for brolucizumab (dashed line) and aflibercept (solid line). FIG. 15B: Free VEGF in the retina.

FIG. 16. Graphs of model simulations performed as described in FIG. 15 but without the loading doses of aflibercept. FIG. 16 shows simulation results in which the administration of aflibercept (following approximately one year of treatment with brolucizumab) omits the loading dose period and instead consists of one dose every 8 weeks starting at Week 56. FIG. 16A: drug PK in the vitreous for brolucizumab (dashed line) and aflibercept (solid line). FIG. 16B: Free VEGF in the retina.

FIG. 17. Graphs of model simulations performed as described in FIG. 15 but with a brolucizumab dosing interval of Q12W during the maintenance period. The last administration of brolucizumab is on Week 44. FIG. 17A: drug PK in the vitreous for brolucizumab (dashed line) and aflibercept (solid line). FIG. 11B: Free VEGF in the retina is shown to rapidly decrease following the first drug administration and to incompletely recover before each of the subsequent doses. The timing and degree of the recovery for free VEGF in the retina.

FIG. 18. Graphs of model simulations performed as described in FIG. 17 but without the loading doses of aflibercept. FIG. 18A: drug PK in the vitreous for brolucizumab (dashed line) and aflibercept (solid line). FIG. 18B: Free VEGF in the retina.

FIG. 19. Graphs of model simulations performed to assess the duration of time that free VEGF in the retina is maintained below indicated threshold concentrations of 5 pM and 10 pM following the last dose of brolucizumab. Brolucizumab was administered by IVT at a dose of 2.0 mg with a regimen consisting of a loading period (one dose every 4 weeks for the first three months) and a maintenance period (one dose every 8 weeks) between Weeks 0 and 48. The timing and degree of the recovery for free VEGF in the retina is shown to vary based upon the level of VEGF production in the eye (exemplified as Low, Mean, or High levels of VEGF production).

FIG. 20. Graphs of model simulations performed as described in FIG. 16 but with the first dose of aflibercept at Day 67 following the last dose of brolucizumab and only under the condition of high VEGF production. This graph is intended to illustrate drug switching to aflibercept under the simulation conditions in which free VEGF in the retina is first observed to surpass the indicated 5 pM threshold following the last dose of brolucizumab (as shown in FIG. 19). FIG. 20A: drug PK in the vitreous for brolucizumab (dashed line) and aflibercept (solid line). FIG. 20B: Free VEGF in the retina.

FIG. 21. Graphs of model simulations in which brolucizumab is administered for a period of approximately one year by IVT at a dose of 6.0 mg with a regimen consisting of one dose every 8 weeks (FIGS. 21A and C) or one dose every 12 weeks (FIGS. 21B and D) between Weeks 0 and 52. FIGS. 21A and C: drug PK in the vitreous for brolucizumab. FIGS. 21B and D: Free VEGF in the retina. The timing and degree of the recovery for free VEGF in the retina is shown to vary based upon the level of VEGF production in the eye (exemplified as Low, Mean, or High levels of VEGF production).

### DETAILED DESCRIPTION

### Definitions

Unless defined otherwise, all technical and scientific terms used herein have the same meaning as commonly understood by those of ordinary skill in the art to which this present disclosure pertains. Any references cited herein, including, e.g., all patents, published patent applications, and non-patent publications, are incorporated by reference in their entirety. To facilitate understanding of the disclosure, several terms and abbreviations as used herein are defined below as follows:

As used herein, all percentages are percentages by weight, unless stated otherwise.

As used herein and unless otherwise indicated, the terms “a” and “an” are taken to mean “one”, “at least one” or “one or more”. Unless otherwise required by context, singular terms used herein shall include pluralities and plural terms shall include the singular.

As used herein, the term “about” includes and describes the value or parameter per se.

For example, “about x” includes and describes “x” per se. As used herein, the term “about” when used in association with a measurement, or used to modify a value, a unit, a constant, or a range of values, refers to variations of 1-10% in addition to including the value or parameter per se. In some embodiments, the term “about” when used in association with a measurement, or used to modify a value, a unit, a constant, or a range of values, refers to variations of ±1, +2, +3, ±4, ±5, ±6, ±7, ±8, +9, or ±10%.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors. The term “VEGF”, in particular, refers to the human VEGF.

The term “VEGF receptor” or “VEGFr” refers to a cellular receptor for VEGF, ordinarily a cell-surface receptor found on vascular endothelial cells, as well as variants thereof retaining the ability to bind hVEGF. One example of a VEGF receptor is the fins-like tyrosine kinase (fit), a transmembrane receptor in the tyrosine kinase family. DeVries et al., Science 255:989 (1992); Shibuya et al., Oncogene 5:519 (1990). The fit receptor comprises an extracellular domain, a transmembrane domain, and an intracellular domain with tyrosine kinase activity. The extracellular domain is involved in the binding of VEGF, whereas the intracellular domain is involved in signal transduction. Another example of a VEGF receptor is the flk-1 receptor (also referred to as KDR). Matthews et al., Proc. Nat. Acad. Sci. 88:9026 (1991); Terman et al., Oncogene 6:1677 (1991); Terman et al., Biochem. Biophys. Res. Commun. 187:1579 (1992). Binding of VEGF to the fit receptor results in the formation of at least two high molecular weight complexes, having an apparent molecular weight of 205,000 and 300,000 Daltons. The 300,000 Dalton complex is believed to be a dimer comprising two receptor molecules bound to a single molecule of VEGF.

As used herein, a “Compound A” is a VEGF antagonist. As used herein, a “Compound B” is a VEGF antagonist. As used herein, Compound A is a VEGF antagonist different from Compound B. The terms VEGF antagonist A, VEGF antagonist “A” and Compound A are used herein interchangeably. The terms VEGF antagonist B, VEGF antagonist “B” and Compound B are used herein interchangeably.

As used herein, a “VEGF antagonist”, including “VEGF antagonist A” and “VEGF antagonist B”, refers to a compound that can diminish or inhibit VEGF activity in vivo. A VEGF antagonist can bind to a VEGF receptor(s) or block VEGF protein(s) from binding to VEGF receptor(s). A VEGF antagonist (e.g., VEGF antagonist A (or VEGF antagonist “A”, or a Compound A), VEGF antagonist B (or VEGF antagonist “B”, or a Compound B)), can be, for example, a small molecule, an anti-VEGF antibody or antigen-binding fragments thereof, fusion protein (such as aflibercept or other such soluble decoy receptor), an aptamer, an antisense nucleic acid molecule, an interfering RNA, receptor proteins, and the like that can bind specifically to one or more VEGF proteins or one or more VEGF receptors. Several VEGF antagonists are described in WO 2006/047325. In one embodiment, the VEGF antagonist (e.g., VEGF antagonist A or VEGF antagonist “A” or also referred herein as Compound A, VEGF antagonist B or VEGF antagonist “B” or also referred herein as Compound B) is any licensed anti-VEGF drug such as brolucizumab, ranibizumab or aflibercept. In one embodiment, the VEGF antagonist (e.g., VEGF antagonist A or VEGF antagonist “A” or Compound A, VEGF antagonist B or VEGF antagonist “B” or Compound B) is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept).

The term “antibody” as used herein includes whole antibodies and any antigen binding fragment (i.e., “antigen-binding portion”, “antigen binding polypeptide”, or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “single chain antibody”, “single chain Fv” or “scFv” is intended to refer to a molecule comprising an antibody heavy chain variable domain (or region; V_{H}) and an antibody light chain variable domain (or region; V_{L}) connected by a linker. Such scFv molecules can have the general structures: NH2-VL-linker-VH-COOH or NH2-VH-linker-VL-COOH.

The term “antigen-binding portion” of an antibody (or simply “antibody portion”) refers to one or more fragments of an antibody that retain the ability to specifically bind to an antigen (e.g., VEGF). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a single domain or dAb fragment (Ward et al., (1989) Nature 341:544-546), which consists of a VH domain; and (vi) an isolated complementarity determining region (CDR) or (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE, or IgM antibody.

As used herein, a “mammal” includes any animal classified as a mammal, including, but not limited to, humans, domestic animals, farm animals, and companion animals, etc.

As used herein, the term “subject” or “patient” refers to human and non-human mammals, including but, not limited to, primates, pigs, horses, dogs, cats, sheep, and cows. Preferably, a subject or patient is a human. In certain embodiments, a patient is at least 18 years of age. In certain embodiments, a patient is at least 50 years of age. In certain embodiments, a patient is at least 65 years of age.

The term “treat”, “treating” or “treatment” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors. As used herein, the terms “treat”, “treatment” and “treating” refer to the reduction or amelioration of the progression or severity of an ocular disease or the amelioration of one or more symptoms, suitably of one or more discernible symptoms of an ocular disease. In specific embodiments, the terms “treat”, “treatment” and “treating” refer to the amelioration of at least one measurable physical parameter of an ocular disease (such as achieve or at least partially achieve a desired effect (e.g. the partial or complete regression of retinal neovascularization, decrease of retinal fluid or achieving retinal fluid-free status, e.g., intraretinal fluid (IRF) and subretinal fluid (SRF), decrease of Central Subfield Thickness (CSFT), improvement in vision, e.g., a change of BCVA>1, >2, >3, >4 or >5 letters, or a DRSS score<61), wherein the physical parameter is not necessarily discernible by the patient.

A term “ocular disease” or “neovascular ocular disease”, as used herein, refers to a condition, disease, or disorder associated with ocular neovascularization. An “ocular disease” or “neovascular ocular disease” that can be treated using a method of the disclosure includes, a condition, disease, or disorder associated with ocular neovascularization, including, but not limited to, abnormal angiogenesis, choroidal neovascularization (CNV), choroidal neovascularization (CNV) associated with nAMD, retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

As used herein, the term “naïve patient” refers to a patient that was not previously treated with one or more doses of a VEGF antagonist.

As used herein, the term “pretreated patient” or “pre-treated patient” or “previously treated patient” refers to a patient previously treated with one or more doses of a VEGF antagonist, e.g., a patient pretreated with one or more doses of a VEGF antagonist different from the VEGF antagonist administered to the patient according to the methods and uses of the present invention. Suitably, the pretreated patient of the disclosure is a non-naïve patient for a treatment with a VEGF antagonist. In particular, the term “pretreated patient” or “pre-treated patient” or “previously treated patient”, as used herein, refers to a patient who was previously administered one or more doses of a VEGF antagonist B (Compound B) and was discontinued from the treatment with the VEGF antagonist B (Compound B).

In specific embodiments, pretreated patient is a patient switched from one VEGF antagonist, e.g., a VEGF antagonist B, to another, e.g., a VEGF antagonist A. As used herein, the term “a patient switched from a therapy with a VEGF antagonist B to a therapy with VEGF antagonist A” refers to a patient who was previously administered one or more doses of a VEGF antagonist B and was discontinued from the treatment with the VEGF antagonist B and is instead administered one or more doses of a VEGF antagonist A. As used herein, the term “a patient switched from a therapy with a Compound B to a therapy with a Compound A” refers to a patient who was previously administered one or more doses of the Compound B and was discontinued from the treatment with the Compound B and is instead administered one or more doses of the Compound A, wherein the Compound B and Compound A are VEGF antagonists, and wherein the Compound B is not the same as the Compound A.

The term “loading phase” refers to the first 2, 3, 4, 5, or 6 doses of a VEGF antagonist administered at q4w or q6w intervals. Suitably, the term “loading phase” refers to the first 2 to 3 doses of a VEGF antagonist administered at q4w.

The term “maintenance phase” refers to additional doses at 4, 6, 7, 8, 9, 10, 11, 12, 13, 14, 15, 16, 17, 18, 19, 20, 21, 22, 23, or 24 week intervals, and can be adjusted as described herein based on Disease Activity Assessments as described herein. Suitably, the term “maintenance phase” refers to additional doses at 8, 9, 10, 11, 12, 13, 14, 15, 16, 17, 18, 19, 20, 21, 22, 23, or 24 week intervals, and can be adjusted as described herein based on Disease Activity Assessments as described herein.

As used herein, an administration interval can be referred to as qXw, where the “X” is a number of weeks between administered doses. For example, q6w is an interval of 6 weeks.

As used herein, the term “week” means 7 days±1 day. As used herein, the term “month” means 25 to 31 days. Also, as used herein, the term “month” means 4 weeks.

As used herein, the terms “effective amount” or “therapeutically effective amount” or “therapeutically effective dose” refer to an amount of a therapy (e.g., a VEGF antagonist, e.g., a VEGF antagonist A, e.g., brolucizumab, or a pharmaceutical composition provided herein) which is sufficient to reduce and/or ameliorate the severity of a given condition, disorder, or disease and/or a symptom related thereto. The term “effective amount” or “therapeutically effective amount” or “therapeutically effective dose” is defined as an amount sufficient to achieve or at least partially achieve a desired effect (e.g. the partial or complete regression of retinal neovascularization, decrease of retinal fluid or achieving retinal fluid-free status, e.g., intraretinal fluid (IRF) and subretinal fluid (SRF), decrease of Central Subfield Thickness (CSFT), improvement in vision, e.g., a change of BCVA>1, >2, >3, >4 or >5 letters, or a DRSS score<61). A therapeutically effective dose is sufficient if it can produce even an incremental change in the symptoms or conditions associated with the disease. The therapeutically effective dose does not have to completely cure the disease or completely eliminate symptoms. Preferably, a therapeutically effective dose can at least partially arrest the disease and/or its complications in a patient already suffering from the disease. In specific embodiments, a therapeutic effective dose may involve repeated administration over a period of time. Amounts effective for this use will depend upon the severity of the disorder being treated and the general state of the patient's own immune system.

### Treatment Regimen for Naïve or Pretreated Patient

In one aspect, the present disclosure provides a method for treating ocular disease, e.g., neovascular age-related macular degeneration (nAMD), in a patient, e.g., a naïve patient or a pretreated patient, the method comprising (consisting of) administering to the patient an initial dose of a VEGF antagonist, e.g., brolucizumab, followed by one or more additional doses of the VEGF antagonist, wherein the one or more additional doses of the VEGF antagonist are administered at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks. In particular, the methods of the present disclosure do not comprise administering to the patient one or more additional doses of the VEGF antagonist in an administration interval of less than 8 weeks.

In one aspect, the present disclosure provides methods for treating ocular disease, e.g., nAMD, in a patient, the method comprising administering to the patient an initial dose of a VEGF antagonist, e.g., brolucizumab, followed by a second dose of the VEGF antagonist at least 8 weeks after the initial dose, e.g., 8, 9, 10, 11, 12, 13, 14, 15, 16, 17, 18, 19, 20, 21, 22, 23, or 24 weeks. Suitably, the methods of the present disclosure further comprise administering one or more additional doses of the VEGF antagonist after the second dose, wherein each additional dose is administered in an administration interval of at least 8 weeks after the immediately preceding dose, e.g., 8, 9, 10, 11, 12, 13, 14, 15, 16, 17, 18, 19, 20, 21, 22, 23, or 24 weeks.

In one aspect, the present disclosure provides methods for treating ocular disease, e.g., nAMD, in a patient, the method comprising (consisting of) administering to the patient one initial dose of a VEGF antagonist, e.g., brolucizumab, followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals. In some embodiments, the maintenance regimen of the VEGF antagonist consists of 2, 3, 4, 5, 6 or more doses administered in an administration interval of at least 8 weeks, e.g., 8, 9, 10, 11, 12, 13, 14, 15, 16, 17, 18, 19, 20, 21, 22, 23, or 24 week intervals. Suitably, the methods of the present disclosure do not comprise administering to the patient one or more additional doses of the VEGF antagonist in an administration interval according to a loading regimen of the VEGF antagonist for the treatment of the ocular disease, in particular wherein the loading regimen of the VEGF antagonist consists of 2, 3, 4, 5, or 6 doses of the VEGF antagonist administered at q4w or q6w intervals.

In one aspect, the present disclosure provides methods for increasing Best Corrected Visual Acuity (BCVA), e.g., for increasing BCVA by ≥5 letters, in a patient, e.g., a naïve patient, wherein the method comprises (consisting of):
(i) administering to the patient an initial dose of a VEGF antagonist (e.g., brolucizumab) followed by one or more additional doses of the VEGF antagonist administered in an administration interval of at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks; or
(ii) administering to the patient an initial dose of a VEGF antagonist (e.g., brolucizumab) followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals,
   and optionally wherein said method provides increasing BCVA by ≥5 letters after the last administration of the VEGF antagonist compared to a baseline BCVA, wherein the baseline BCVA was assessed prior to the first administration of the VEGF antagonist.

In one aspect, the present disclosure provides methods for increasing Visual Acuity (VA) in a patient, e.g., a naïve patient, wherein the method comprises (consisting of):
(i) administering to the patient an initial dose of a VEGF antagonist (e.g., brolucizumab) followed by one or more additional doses of the VEGF antagonist administered in an administration interval of at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks; or
(ii) administering to the patient an initial dose of a VEGF antagonist (e.g., brolucizumab) followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals,
   and optionally wherein said method provides increasing VA after the last administration of the VEGF antagonist compared to a baseline VA, wherein the baseline VA was assessed prior to the first administration of the VEGF antagonist.

In one aspect, the present disclosure provides methods for decreasing or reducing Central Subfield Thickness (CSFT) in a patient, e.g., a naïve patient, wherein the method comprises (consisting of):
(i) administering to the patient an initial dose of a VEGF antagonist (e.g., brolucizumab) followed by one or more additional doses of the VEGF antagonist administered in an administration interval of at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks; or
(ii) administering to the patient an initial dose of a VEGF antagonist (e.g., brolucizumab) followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals,
   and optionally wherein said method provides decreasing or reducing CSFT after the last administration of the VEGF antagonist compared to a baseline CSFT, wherein the baseline CSFT was assessed prior to the first administration of the VEGF antagonist.

In one aspect, the present disclosure provides methods for decreasing or reducing Intraretinal Cysts (IRC) and/or Intraretinal Fluid (IRF) and/or Subretinal Fluid (SRF) in a patient, e.g., a naïve patient, wherein the method comprises (consisting of):
(i) administering to the patient an initial dose of a VEGF antagonist (e.g., brolucizumab) followed by one or more additional doses of the VEGF antagonist administered in an administration interval of at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks; or
(ii) administering to the patient an initial dose of a VEGF antagonist (e.g., brolucizumab) followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals,
   and optionally wherein said method provides decreasing or reducing IRC and/or IRF and/or SRF after the last administration of the VEGF antagonist (e.g., brolucizumab) compared to a baseline IRC and/or IRF and/or SRF, wherein the baseline IRC and/or IRF and/or SRF was assessed prior to the first administration of the VEGF antagonist.

In one aspect, the present disclosure provides methods of inhibiting VEGF or suppression of VEGF levels in retina of a patient, e.g., a naïve patient, wherein the method comprises (consisting of):
(i) administering to the patient an initial dose of a VEGF antagonist (e.g., brolucizumab) followed by one or more additional doses of the VEGF antagonist administered in an administration interval of at least 8 weeks, e.g., 8, 9, 10, 11, 12, 13, 14, 15, 16, 17, 18, 19, 20, 21, 22, 23, or 24 weeks, after the initial dose and each of the one or more additional doses after the initial dose are administered in an administration interval of at least 8 weeks, e.g., 8, 9, 10, 11, 12, 13, 14, 15, 16, 17, 18, 19, 20, 21, 22, 23, or 24 weeks; or
(ii) administering to the patient an initial dose of a VEGF antagonist (e.g., brolucizumab) followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals,
   and optionally wherein the patient treated with the methods of the present disclosure has retinal levels of free-VEGF concentration below a target threshold of 10 pM, preferably below a target threshold of 5 pM, over at least 85% (e.g., at least 90%, at least 95%, preferably 100%) of the duration of the treatment period. In a specific embodiment, the treatment period is at least 6 months, preferably at least 12 months or 52 weeks.

In a further aspect, the present disclosure provides a VEGF antagonist, e.g., brolucizumab, for use as a medicament for treating ocular disease, e.g., nAMD, in a patient, wherein the VEGF antagonist is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present disclosure provides a VEGF antagonist, e.g., brolucizumab, for use as a medicament for treating ocular disease, e.g., nAMD, in a patient, wherein the VEGF antagonist is administered to the patient as an initial dose followed by one or more additional doses, wherein the one or more additional doses of the VEGF antagonist are administered at least 8 weeks after the initial dose and each of the one or more additional doses after the initial dose are administered in an administration interval of at least 8 weeks.

In another aspect, the present disclosure provides a VEGF antagonist, e.g., brolucizumab, for use as a medicament for treating ocular disease, e.g., nAMD, in a patient, wherein the VEGF antagonist is administered to the patient as one initial dose followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

In a further aspect, the present disclosure provides a pharmaceutical composition comprising a VEGF antagonist, e.g., brolucizumab, for use as a medicament for treating ocular disease, e.g., nAMD, in a patient, wherein the pharmaceutical composition is administered to the patient as an initial dose followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present disclosure provides a pharmaceutical composition comprising a VEGF antagonist, e.g., brolucizumab, for use as a medicament for treating ocular disease, e.g., nAMD, in a patient, wherein the pharmaceutical composition is administered to the patient as an initial dose followed by one or more additional doses, wherein the one or more additional doses of the pharmaceutical composition are administered at least 8 weeks after the initial dose and each of the one or more additional doses after the initial dose are administered in an administration interval of at least 8 weeks.

In another aspect, the present disclosure provides a pharmaceutical composition comprising a VEGF antagonist, e.g., brolucizumab, for use as a medicament for treating ocular disease, e.g., nAMD, in a patient, wherein the pharmaceutical composition is administered to the patient as one initial dose followed by a maintenance regimen of additional doses of the pharmaceutical composition administered in an administration interval of at least 8 weeks, e.g., 8, 9, 10, 11, 12, 13, 14, 15, 16, 17, 18, 19, 20, 21, 22, 23, or 24 week intervals.

In a further aspect, the present disclosure provides use of a VEGF antagonist, e.g., brolucizumab, for the manufacture of a medicament for treating ocular disease, e.g., nAMD, in a patient, the use comprising administering to the patient an initial dose of the VEGF antagonist followed by a second dose at least 8 weeks after the initial dose, e.g., 8, 9, 10, 11, 12, 13, 14, 15, 16, 17, 18, 19, 20, 21, 22, 23, or 24 weeks.

In another aspect, the present disclosure provides use of a VEGF antagonist, e.g., brolucizumab, for the manufacture of a medicament for treating ocular disease, e.g., nAMD, in a patient, the use comprising administering to the patient an initial dose of the VEGF antagonist followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

In another aspect, the present disclosure provides use of a VEGF antagonist, e.g., brolucizumab, for the manufacture of a medicament for treating ocular disease, e.g., nAMD, in a patient, the use comprising administering to the patient one initial dose of the VEGF antagonist followed by a maintenance regimen of additional doses of the VEGF antagonist administered in an administration interval of at least 8 weeks, e.g., 8, 9, 10, 11, 12, 13, 14, 15, 16, 17, 18, 19, 20, 21, 22, 23, or 24 week intervals.

In one embodiment, the ocular disease is selected from the list consisting of abnormal angiogenesis, choroidal neovascularization (CNV), choroidal neovascularization (CNV) associated with nAMD, retinal vascular permeability, retinal edema, diabetic retinopathy (e.g., proliferative diabetic retinopathy (PDR) and non-proliferative diabetic retinopathy (NPDR)), macular edema (ME), diabetic macular edema (DME), neovascular (exudative) age-related macular degeneration (nAMD), sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization. In one embodiment, the disease is diabetic macular edema (DME). In one embodiment, the disease is diabetic retinopathy (DR). In a one embodiment, the disease is proliferative diabetic retinopathy (PDR). In a one embodiment, the disease is non-proliferative diabetic retinopathy (NPDR). In one embodiment, the disease is Retinal Vein Occlusion (RVO), e.g., Central Retinal Vein Occlusion (CRVO), e.g., Branch Retinal Vein Occlusion (BRVO). In a preferred embodiment, the disease is nAMD.

The methods and uses of the present disclosure are suitable for a naïve patient, e.g., a patient that has not previously received one or more doses of a VEGF antagonist. The methods and uses of the present disclosure also are suitable for a pretreated patient, e.g., a patient previously treated with one or more doses of a VEGF antagonist different from the VEGF antagonist administered to the patient according to the methods and uses of the present disclosure.

In some embodiments, the methods and uses of the present disclosure comprise administering to a patient an initial dose of a VEGF antagonist, e.g., brolucizumab, followed by one or more doses of the VEGF antagonist, wherein the patient does not have (i) ocular inflammation, e.g., active ocular inflammation, and/or (ii) retinal vasculitis and/or retinal vascular occlusion, e.g., retinal vasculitis and/or retinal vascular occlusion in the presence of intraocular inflammation.

In some embodiments, according to the methods or the uses of the present disclosure, the initial dose of the VEGF antagonist is followed by one or more doses of the VEGF antagonist in an administration interval as individualized by a physician based on a disease activity assessment and/or in an administration interval of at least 8 weeks, e.g., 8, 9, 10, 11, 12, 13, 14, 15, 16, 17, 18, 19, 20, 21, 22, 23, or 24 weeks. In some embodiments, according to the methods or the uses of the present disclosure, the initial dose of the VEGF antagonist is followed by one or more doses of the VEGF antagonist in an administration interval as individualized by a physician based on a disease activity assessment and/or in an administration interval between ≥8 and ≤24 weeks, e.g., between 8 and ≤18 weeks (≥q8w to ≤q18w), between ≥8 and ≤12 weeks (≥q8w to ≤q12w). Suitably, the initial dose of the VEGF antagonist is followed by administering to the patient one or more doses of the VEGF antagonist once every 8 weeks (q8w regimen) or once every 12 weeks (q12w regimen) and/or as individualized by a physician based on a disease activity assessment.

In certain embodiments, an initial dose of a VEGF antagonist (e.g., brolucizumab) is followed by one or more doses of the VEGF antagonist in an administration interval, e.g., an injection interval, of at least about two months, e.g., at least about three months, at least about four months, at least about five months, at least about six months. In a preferred embodiment, an initial dose of a VEGF antagonist (e.g., brolucizumab) is followed by one or more doses of the VEGF antagonist in an administration interval, e.g., an injection interval, of at least about two months. In a more preferred embodiment, an initial dose of a VEGF antagonist (e.g., brolucizumab) is followed by one or more doses of the VEGF antagonist in an administration interval, e.g., an injection interval, of at least about three months. Suitably, an initial dose of a VEGF antagonist (e.g., brolucizumab) is followed by one or more doses of the VEGF antagonist in an administration interval, e.g., an injection interval, between about 2 months and about 6 months (e.g., about 2 months and about 3 months), more preferably between about 3 months and about 6 months.

Suitably, the methods and the uses of the disclosure comprise (consist of) administering to a patient an initial administration, e.g., injection, of a VEGF antagonist, e.g., brolucizumab, followed by subsequent administrations, e.g., injections with the VEGF antagonist once every 8 weeks (2 months) to 12 weeks (3 months) and/or as individualized by a physician based on a disease activity assessment. In a specific embodiment the methods and the uses of the disclosure comprise (consist of) administering to a patient an initial administration, e.g., injection, of a VEGF antagonist, e.g., brolucizumab, followed by subsequent administrations, e.g., injections with the VEGF antagonist once every 12 weeks (3 months).

Suitably, the disclosure provides the methods and the uses, wherein when starting a patient on a VEGF antagonist, e.g., brolucizumab, no loading of the VEGF antagonist (e.g., 3 monthly injections or three q4w injections or three q6w injections) is needed. Suitably, the administration regiment of the VEGF antagonist of the methods and the uses of the disclosure does not need a loading phase of the VEGF antagonist (e.g., 3 monthly injections or three q4w injections or three q6w injections). Suitably, an initial dose of a VEGF antagonist (e.g., brolucizumab) is followed by one or more doses of the VEGF antagonist with an administration interval according to a maintenance phase regimen of the VEGF antagonist for the treatment of the ocular disease, e.g., nAMD, e.g., as specified on the corresponding drug label for the VEGF antagonist. Suitably, an initial dose of a VEGF antagonist (e.g., brolucizumab) is followed by one or more doses of the VEGF antagonist with an administration interval according to a maintenance phase regimen of the VEGF antagonist for the treatment of the ocular disease, e.g., nAMD, and without a loading phase of the VEGF antagonist (e.g., without the first three monthly injections of the VEGF antagonist).

In some embodiments, the methods and the uses of the disclosure comprise (consist of) administering to a patient an initial dose of a VEGF antagonist, e.g., brolucizumab, followed by one or more doses of the VEGF antagonist in an administration interval, e.g., an injection interval, as describe herein and without a loading phase of the VEGF antagonist.

In certain embodiments, a patient receives an initial injection of a VEGF antagonist followed by an additional injection about every 12 weeks (q12w) of the VEGF antagonist. In certain embodiments, a patient, e.g., a naïve patient, receives an initial injection of a VEGF antagonist followed by an additional injection about every 8 weeks (q8w) of the VEGF antagonist. The administration scheme of the VEGF antagonist as disclosed represents the maintenance phase of the VEGF antagonist. The maintenance phase can include administering additional doses at about 8-week, about 9-week, about 10-week, about 11-week, about 12-week, about 13-week, about 14-week, about 15-week, about 16-week, about 17-week, about 18-week, about 19-week, about 20-week, about 21-week, about 22-week, about 23-week, or about 24-week intervals, including a combination of such intervals. The maintenance phase can include administering additional doses at about 2 months, about 3 months, about 4 months, about 5 months, about 6 months intervals, including a combination of such intervals.

In one embodiment, the methods and uses of the disclosure comprise (consist of) administering to a patient an initial dose of a VEGF antagonist, e.g., brolucizumab, followed by one or more doses of the VEGF antagonist according to a “maintenance phase” schedule of the VEGF antagonist, e.g., doses of the VEGF antagonist administered at about 8 to about 12-week intervals, e.g., about 8 week intervals or about 12 week intervals, or at about 2 months to about 3 months intervals, e.g., about 2 months intervals or about 3 months intervals.

In a specific embodiment, the present disclosure provides methods and uses for treating nAMD in a patient the method or the use comprises (consists of) administering to the patient an initial dose of a VEGF antagonist, e.g., brolucizumab, followed by one or more doses of the VEGF antagonist with an administration interval between about 8 and about 12 weeks. In specific embodiment, the VEGF antagonist is brolucizumab. In a more specific embodiment, the dose of the VEGF antagonist (e.g., an initial dose and following doses) is from about 3 mg to about 6 mg, e.g., about 3 mg or about 6 mg, preferably 6 mg.

In one embodiment, the present disclosure provides methods and uses for treating diabetic macular edema (DME) in a patient the method or the use comprises (consists of) administering to the patient an initial dose of a VEGF antagonist, e.g., brolucizumab, followed by one or more doses of the VEGF antagonist with an administration interval between about 8 and about 12 weeks. In a specific embodiment, the VEGF antagonist is brolucizumab. In a more specific embodiment, wherein the dose of the VEGF antagonist (e.g., an initial dose and following doses) is from about 3 mg to about 6 mg, e.g., about 3 mg or about 6 mg, preferably 6 mg.

In another embodiment, the present disclosure provides methods and uses for treating nAMD in a patient, the method or the use comprises (consists of) administering to the patient an initial dose of a VEGF antagonist, e.g., aflibercept, followed by one or more doses of the VEGF antagonist with an administration interval between about 8 and about 12 weeks, preferably 8 weeks. Suitably, the VEGF antagonist is aflibercept. Suitably, the VEGF antagonist is aflibercept and the dose of the VEGF antagonist (e.g., an initial dose and following doses) is about 2 mg.

In certain embodiment, the maintenance phase starts with a dosing regimen wherein a VEGF antagonist, e.g., brolucizumab, is administered once every 12 weeks (q12w), and the dosing interval is adjusted (e.g., plus or minus 2, 3, 4, 5, 6, 7, 8, 10, 11 or 12 weeks) depending on a disease activity assessment. For example, if disease activity is observed prior to administering a q12w dose, the patient will receive the q12w dose as planned, and receive the next dose 8 weeks later, thus being placed on a q8w dosing regimen until disease activity is no longer observed. When disease activity is no longer observed, the dosing regimen will be adjusted back to a q12w schedule. In another embodiment, if no disease activity is observed at any time during the maintenance phase, the treatment interval may be extended by 4 weeks to a q16w. If disease activity is observed in a patient on a q16w or other dosing regimen more than q12w, the treatment interval may be adjusted back to a q12w dosing regimen.

In certain embodiments, a Disease Activity Assessment (DAA) is conducted at all scheduled treatment visits. In some embodiments, the methods or the uses of the present disclosure comprise assessing the patient for ocular disease activity before or after administering a dose of the VEGF antagonist. In some embodiments, the methods or the uses of the present disclosure comprise assessing the patient for ocular disease activity before or after administering every q8w or q12w dose of the VEGF antagonist.

At assessment weeks, a patient can be currently on, for example, an 8-week or 12-week or 16-week interval regimen. The assessment can determine if a patient stays on the current interval or switches to a different interval. For example, a patient is reassigned to q8w or q12w or q16w dosing regimen based on the presence of the disease activity as determined by a treatment provider.

Suitably, the disease activity may be assessed based on visual function, retinal structure and leakage. An assessment as described herein preferably includes one or more of the following tests to assess activity of a VEGF antagonist (e.g., brolucizumab) on visual function, retinal structure and leakage: (i) best corrected visual acuity (BCVA), e.g., best corrected visual acuity with ETDRS-like chart at 4 meters, (ii) visual acuity (VA), (iii) central subfield thickness (CSFT), (iv) presence of intraretinal cysts/fluid, (v) ETDRS DRSS score based on 7-field stereo Color Fundus Photography (CFP), (vi) anatomical retinal evaluation by Optical Coherence Tomography (OCT), standard or wide-field Fluorescein Angiography (FA), OCT angiography, and/or wide-field CFP/FA, (vii) peripheral visual field assessed by perimetry, (viii) contrast sensitivity, (viii) severity of diabetic retinopathy.

Visual acuity can be assessed using best correction determined from protocol refraction (BCVA). BCVA measurements can be taken, for example, in a sitting position using ETDRS-like visual acuity testing charts.

Optical Coherence Tomography (OCT), color fundus photography and fluorescein angiography can be assessed according to methods known to those of skill in the art.

The CST is the average thickness of circular 1 mm area centered around the fovea measured from retinal pigment epithelium (RPE) to the internal limiting membrane (ILM), inclusively. CST can be measured, for example, using spectral domain Optical Coherence Tomography (SD-OCT).

Means of performing the above tests are well understood and commonly used by those skilled in the art.

Suitably, the disease activity may be assessed based on one or more of the following: (i) best corrected visual acuity (BCVA), (ii) visual acuity (VA), (iii) central subfield thickness (CSFT), and (iv) presence of intraretinal cysts/fluid. The presence of ocular disease activity, e.g., nAMD disease activity, includes one or more of the following: (i) decrease in Best Corrected Visual Acuity (BCVA), (ii) decrease in Visual Acuity (VA), (iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF). Fluid measured in the eye can be intraretinal and/or subretinal fluid.

In specific embodiments, the presence of ocular disease activity, e.g., nAMD disease activity, includes one or more of the following:
(i) decrease in BCVA of ≥2 letters, e.g., decrease in BCVA of ≥3 letters, decrease in BCVA of ≥4 letters, in particular decrease in BCVA of ≥5 letters, more particularly wherein:
   the decrease in BCVA is observed at Week 8 or Week 12 after the last administration of the VEGF antagonist (e.g., brolucizumab) compared to a baseline BCVA, wherein the baseline BCVA was assessed prior to the last administration of the VEGF antagonist, or
   the decrease in BCVA is observed after 4 months or longer, e.g., after 6 months or longer, of the administration of the VEGF antagonist compared to a baseline BCVA, wherein the baseline BCVA was assessed 4 months or longer, e.g., 6 months or longer, prior to the last administration of the VEGF antagonist;
(ii) decrease in VA of ≥1 letters, e.g., decrease in VA of ≥2 letters, in particular decrease in VA of ≥3 letters, more particularly wherein:
   the decrease in VA is observed at Week 8 or Week 12 after the last administration of the VEGF antagonist (e.g., brolucizumab) compared to a baseline VA, wherein the baseline VA was assessed prior to the last administration of the VEGF antagonist, or
   the decrease in VA is observed after 4 months or longer, e.g., after 6 months or longer, of the administration of the VEGF antagonist compared to a baseline VA, wherein the baseline VA was assessed 4 months or longer, e.g., 6 months or longer, prior to the last administration of the VEGF antagonist;
(iii) CSFT increase ≥25 μm, e.g., CSFT increase ≥50 μm, in particular CSFT increase ≥75 μm, more particularly wherein:
   the CSFT increase is observed at Week 8 or Week 12 after the last administration of the VEGF antagonist (e.g., brolucizumab) compared to a baseline CSFT, wherein the baseline CSFT was assessed prior to the last administration of the VEGF antagonist, or
   the CSFT increase is observed after 4 months or longer, e.g., after 6 months or longer, of the administration of the VEGF antagonist compared to a baseline CSFT, wherein the baseline CSFT was assessed 4 months or longer, e.g., 6 months or longer, prior to the last administration of the VEGF antagonist;
(iv) new or persistent or recurrent intraretinal cysts (IRC) and/or intraretinal fluid (IRF) and/or subretinal fluid (SRF), in particular wherein:
   new or persistent or recurrent intraretinal cysts (IRC) and/or intraretinal fluid (IRF) and/or subretinal fluid (SRF) are observed at Week 8 or Week 12 after the last administration of the VEGF antagonist (e.g., brolucizumab) compared to a baseline RC and/or IRF and/or SRF, wherein the baseline RC and/or IRF and/or SRF was assessed prior to the last administration of the VEGF antagonist, or
   new or persistent or recurrent intraretinal cysts (IRC) and/or intraretinal fluid (IRF) and/or subretinal fluid (SRF) are observed after 4 months or longer, e.g., after 6 months or longer, of the administration of the VEGF antagonist compared to a baseline IRC and/or IRF and/or SRF, wherein the baseline IRC and/or IRF and/or SRF was assessed 4 months or longer, e.g., 6 months or longer, prior to the last administration of the VEGF antagonist.

Where disease activity is present (for example, loss of letters measured by BCVA, increase in CST, increased fluid accumulation, and or increased severity of ocular disease compared with baseline reading for a patient or compared with any previous assessment), a more frequent dosing interval is prescribed going forward. Where improvement of disease activity is observed, a less frequent dosing interval is prescribed.

Suitably, the methods and uses of the present disclosure comprise administering to a patient an initial dose of a VEGF antagonist followed by one or more doses of a VEGF antagonist according to “treat-to-control” (TtC) dosing regimen concept, e.g., wherein a dosing interval is adjusted based on disease activity to meet the patient's needs, including shorting, maintaining or extending the treatment interval based on disease activity. The Treat-to-Control (TtC) regimen entails sustained disease control to determine the optimal treatment interval for each patient. For example, the Treat-to-Control (TtC) dosing regimen entails increasing or decreasing a dosing interval in 2 weeks or 4 weeks steps.

Suitably, the methods and uses of the present disclosure comprise administering to a patient, e.g., a naïve patient, an initial dose of a VEGF antagonist followed by one or more doses of a VEGF antagonist according to Treat-and-Extend (T&E) dosing regimen concepts (Wykoff et al., 2018). For example, the Treat-and-Extend (T&E) dosing regimen entails increasing or decreasing a dosing interval in 2 weeks steps.

In certain embodiments, the dosing frequency is adjusted based on the outcome of disease activity assessments, for example using pre-defined visual and anatomic criteria. In one embodiment, dosing frequency of a VEGF antagonist (e.g., brolucizumab) can be adjusted by decreasing the dosing interval from once every 24 weeks (q24w) to once every 18 weeks (q18w). In one embodiment, dosing frequency of a VEGF antagonist (e.g., brolucizumab) can be adjusted by decreasing the dosing interval from once every 18 weeks (q18w) to once every 12 weeks (q12w). In one embodiment, dosing frequency of a VEGF antagonist (e.g., brolucizumab) can be adjusted by decreasing the dosing interval from once every 12 weeks (q12w) to once every 8 weeks (q8w) based on the disease activity assessment at any scheduled treatment visit. In another embodiment, dosing frequency of a VEGF antagonist (e.g., brolucizumab) can be adjusted by increasing the dosing interval from once every 8 weeks (q8w) to once every 12 weeks (q12w) based on the disease activity assessment at any scheduled treatment visit. In another embodiment, dosing frequency of a VEGF antagonist (e.g., brolucizumab) can be adjusted by increasing the dosing interval from once every 12 weeks (q12w) or to once every 18 weeks (q18w) or to once every 24 weeks (q24w) based on the disease activity assessment at any scheduled treatment visit. When disease activity is identified as described herein, the treatment regimen can be changed, e.g., from every 12 weeks to every 8 weeks (i.e., q8w). The disclosure provides specific criteria established by the inventors based on disease activity assessments to determine when a shorter administration interval, e.g., an injection interval, should be used and when a longer administration interval, e.g., an injection interval, should be used, e.g., an 8-week interval should be used and when a 12-week interval should be continued. In some cases, a patient might be on a 12-week interval regimen for some time, and then switch to an 8-week interval, and then switch back to the 12-week interval. Thus, patients may not stay on one interval regimen, and may go back and forth depending on assessments according to the criteria set forth herein.

In one embodiment, assessments of disease activity to establish patient's disease status occurs at baseline (e.g., Week 0; first treatment with a VEGF antagonist; prior to the last administration of a VEGF antagonist). The assessment of the disease activity (DAA) during treatment regimens is at the discretion of the person making the assessment (e.g., the treatment provider), and is based on changes in vision and anatomical and morphological and clinical parameters with reference to patients' baseline disease status (e.g., at Week 0; first treatment with a VEGF antagonist; prior to the last administration of a VEGF antagonist).

In certain other embodiments, during the maintenance phase, a VEGF antagonist is administered on an as needed basis, i.e., pro re nata (PRN), at the discretion of a treatment provider (e.g., a physician or other qualified medical professional) based on visual and/or anatomical outcomes to determine disease activity.

In one embodiment, the VEGF antagonist of the disclosure is any licensed anti-VEGF drug such as brolucizumab, ranibizumab or aflibercept. In one embodiment, the VEGF antagonist of the disclosure is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept). In one embodiment, the VEGF antagonist of the disclosure is brolucizumab or ranibizumab or aflibercept. In a preferred embodiment, the VEGF antagonist of the disclosure is brolucizumab.

In certain embodiments, the VEGF antagonist of the disclosure is an anti-VEGF antibody, e.g., a single chain antibody (scFv) or Fab fragment.

In certain embodiments, the VEGF antagonist of the disclosure is an anti-VEGF antibody, e.g., anti-VEGF antibodies described in WO 2009/155724, the entire contents of which are hereby incorporated by reference.

In one embodiment, the VEGF antagonist of the disclosure is an anti-VEGF antibody comprising a variable heavy chain having the sequence as set forth in SEQ ID NO: 1 and a variable light chain having the sequence as set forth in SEQ ID NO: 2.

VH:
SEQ ID NO. 1
EVQLVESGGGLVQPGGSLRLSCTASGFSLTDYYYMTWVRQAPGKGLEWV
GFIDPDDDPYYATWAKGRFTISRDNSKNTLYLQMNSLRAEDTAVYYCAG
GDHNSGWGLDIWGQGTLVTVSS
VL:
SEQ ID NO. 2
EIVMTQSPSTLSASVGDRVIITCQASEIIHSWLAWYQQKPGKAPKLLIY
LASTLASGVPSRFSGSGSGAEFTLTISSLQPDDEATYYCQNVYLASTNG
ANFGQGTKLTVLG

In another embodiment, the VEGF antagonist of the disclosure is an anti-VEGF antibody comprising the sequence as set forth in SEQ ID NO: 3

SEQ ID NO: 3
EIVMTQSPSTLSASVGDRVIITCQASEIIHSWLAWYQQKPGKAPKLLIY
LASTLASGVPSRFSGSGSGAEFTLTISSLQPDDFATYYCONVYLASTNG
ANFGQGTKLTVLGGGGGSGGGGSGGGGSGGGGSEVQLVESGGGLVQPGG
SLRLSCTASGFSLTDYYYMTWVRQAPGKGLEWVGFIDPDDDPYYATWAK
GRFTISRDNSKNTLYLQMNSLRAEDTAVYYCAGGDHNSGWGLDIWGQGT
LVTVSS.

In a preferred embodiment, the VEGF antagonist of the disclosure is brolucizumab (which comprises the sequence of SEQ ID NO: 3). The sequence of brolucizumab is set forth in SEQ ID NO: 4. A methionine derived from the start codon in an expression vector is present in the final protein in cases where it has not been cleaved posttranslationally as follows.

(SEQ ID NO: 4)
MEIVMTQSPS TLSASVGDRV IITCQASEII HSWLAWYQQK
PGKAPKLLIY LASTLASGVP SRFSGSGSGA EFTLTISSLQ
PDDFATYYCQ NVYLASTNGA NFGQGTKLTV LGGGGGSGGG
GSGGGGSGGG GSEVOLVESG GGLVQPGGSL RLSCTASGES
LTDYYYMTWV RQAPGKGLEW VGFIDPDDDP YYATWAKGRF
TISRDNSKNT LYLQMNSLRA EDTAVYYCAG GDHNSGWGLD
IWGQGTLVTV SS

In another embodiment, the VEGF antagonist of the disclosure is an anti-VEGF antibody comprising three light chain CDRs (CDRL1, CDRL2, and CDRL3) and three heavy chain CDRs (CDRH1, a CDRH2, a CDRH3) as follows:

CDRL1
SEQ ID NO: 5
QASEIIHSWLA
CDRL2
SEQ ID NO: 6
LASTLAS
CDRL3
SEQ ID NO: 7
QNVYLASTNGAN
CDRH1
SEQ ID NO: 8
GFSLTDYYYMT
CDRH2
SEQ ID NO: 9
FIDPDDDPYYATWAKG
CDRH3
SEQ ID NO: 10
GDHNSGWGLDI

Brolucizumab, is a humanized single-chain Fv (scFv) antibody fragment inhibitor of VEGF with a molecular weight of ˜26 kDa. It is an inhibitor of VEGF-A and works by binding to the receptor binding site of the VEGF-A molecule, thereby preventing the interaction of VEGF-A with its receptors VEGFR1 and VEGFR2 on the surface of endothelial cells. Increased levels of signaling through the VEGF pathway are associated with pathologic ocular angiogenesis and retinal edema. Inhibition of the VEGF pathway has been shown to inhibit the growth of neovascular lesions and resolve retinal edema in patients with nAMD.

In a preferred embodiment, the VEGF antagonist of the disclosure is brolucizumab. Accordingly, the pretreated patient is a patient pretreated with one or more doses of a VEGF antagonist different from brolucizumab. In some embodiments, the patient was pretreated with a VEGF antagonist selected from the group consisting of aflibercept, ranibizumab, faricimab, conbercept and abicipar.

In certain embodiments, the VEGF antagonist of the disclosure is administered by an injection, e.g., an intravitreal injection.

In some embodiments, the VEGF antagonist of the disclosure is brolucizumab and is administered at a dose of about 1, about 2, about 3, about 4, about 5, or about 6 mg (e.g., about 6 mg/0.05 mL) as an intravitreal injection. In certain embodiments, the VEGF antagonist of the disclosure is brolucizumab and is administered at a dose of 1, 2, 3, 4, 5, or 6 mg (e.g., 6 mg/0.05 mL) as an intravitreal injection.

In some embodiments, the VEGF antagonist of the disclosure is aflibercept and is administered at a dose of about 0.5, about 1 or about 2 mg (e.g., about 2 mg/0.05 mL) as an intravitreal injection. In certain embodiments, the VEGF antagonist of the disclosure is aflibercept and is administered at a dose of 0.5, 1 or 2 mg (e.g., 2 mg/0.05 mL) as an intravitreal injection. Accordingly, the pretreated patient is a patient pretreated with one or more doses of a VEGF antagonist different from aflibercept. In some embodiments, the patient was pretreated with a VEGF antagonist selected from the group consisting of brolucizumab, ranibizumab, faricimab, conbercept and abicipar.

In some embodiments, the VEGF antagonist of the disclosure is ranibizumab and is administered at a dose of about 0.2, about 0.3, about 0.4 or about 0.5 mg (e.g., about 0.5 mg/0.05 mL) as an intravitreal injection. In certain embodiments, the VEGF antagonist of the disclosure is ranibizumab and is administered at a dose of 0.2, 0.3, 0.4 or 0.5 mg (e.g., 0.5 mg/0.05 mL) as an intravitreal injection. Accordingly, the pretreated patient is a patient pretreated with one or more doses of a VEGF antagonist different from ranibizumab. In some embodiments, the patient was pretreated with a VEGF antagonist selected from the group consisting of brolucizumab, aflibercept, faricimab, conbercept and abicipar.

Treatment Regimen for a Patient Switched from One VEGF Antagonist to Another

The disclosure provides a method of administering a VEGF antagonist A (e.g., brolucizumab) for treating ocular disease, in particular neovascular age-related macular degeneration (nAMD), to a patient pretreated with one or more doses of a VEGF antagonist B (e.g., aflibercept or ranibizumab). The disclosure provides a method of administering a Compound A (e.g., brolucizumab) for treating ocular disease, in particular neovascular age-related macular degeneration (nAMD), to a patient pretreated with one or more doses of a Compound B (e.g., aflibercept or ranibizumab), wherein the Compound A and the Compound B are VEGF antagonists, and wherein the Compound A is different from the Compound B.

In one aspect, the present disclosure provides methods for treating ocular disease, in particular neovascular age-related macular degeneration (nAMD), in a patient that has previously received one or more doses of a VEGF antagonist B (e.g., aflibercept, ranibizumab) or in a patient pretreated with one or more doses of a VEGF antagonist B (e.g., aflibercept or ranibizumab), the method comprises administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment. Suitably, the present disclosure provides methods for treating ocular disease, in particular neovascular age-related macular degeneration (nAMD), in a patient that has previously received one or more doses of a Compound B (e.g., aflibercept, ranibizumab) or in a patient pretreated with one or more doses of a Compound B (e.g., aflibercept or ranibizumab), the method comprises administering to the patient an initial dose of a Compound A (e.g., brolucizumab) followed by one or more additional doses of the Compound A, in particular wherein said one or more additional doses of the Compound A are administered in an administration interval of no less than about 8 weeks and/or as individualized by a physician based on a disease activity assessment, wherein the Compound A and the Compound B are VEGF antagonists, and wherein the Compound A is different from the Compound B.

In one aspect, the present disclosure provides methods for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), the method comprises administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease. Suitably, the present disclosure provides methods for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), the method comprises administering to the patient an initial dose of a Compound A (e.g., brolucizumab) followed by one or more additional doses of the Compound A in an administration interval according to a maintenance regimen of the Compound A for the treatment of the ocular disease, wherein the Compound A and the Compound B are VEGF antagonists, and wherein the Compound A is different from the Compound B.

In one aspect, the present disclosure provides methods for increasing Best Corrected Visual Acuity (BCVA), in particular for increasing BCVA by ≥5 letters, in a patient that has previously received one or more doses of a VEGF antagonist B (e.g., aflibercept, ranibizumab) or in a patient pretreated with one or more doses of a VEGF antagonist B (e.g., aflibercept or ranibizumab), wherein the method comprises:
(i) administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment; or
(ii) administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease,
   and in particular wherein said method provides increasing BCVA by ≥5 letters after the last administration of the VEGF antagonist A (e.g., brolucizumab) compared to a baseline BCVA, wherein the baseline BCVA was assessed prior to the first administration of the VEGF antagonist B (e.g., aflibercept or ranibizumab).

In one aspect, the present disclosure provides methods for increasing Visual Acuity (VA) in a patient that has previously received one or more doses of a VEGF antagonist B (e.g., aflibercept, ranibizumab) or in a patient pretreated with one or more doses of a VEGF antagonist B (e.g., aflibercept or ranibizumab), wherein the method comprises:
(i) administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment; or
(ii) administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease,
   and in particular wherein said method provides increasing VA after the last administration of the VEGF antagonist A (e.g., brolucizumab) compared to a baseline VA, wherein the baseline VA was assessed prior to the first administration of the VEGF antagonist B (e.g., aflibercept or ranibizumab).

In one aspect, the present disclosure provides methods for decreasing or reducing Central Subfield Thickness (CSFT) in a patient that has previously received one or more doses of a VEGF antagonist B (e.g., aflibercept, ranibizumab) or in a patient pretreated with one or more doses of a VEGF antagonist B (e.g., aflibercept or ranibizumab), wherein the method comprises:
(i) administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment; or
(ii) administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease,
   and in particular wherein said method provides decreasing or reducing CSFT after the last administration of the VEGF antagonist A (e.g., brolucizumab) compared to a baseline CSFT, wherein the baseline CSFT was assessed prior to the first administration of the VEGF antagonist B (e.g., aflibercept or ranibizumab).

In one aspect, the present disclosure provides methods for decreasing or reducing Intraretinal Cysts (IRC) and/or Intraretinal Fluid (IRF) and/or Subretinal Fluid (SRF) in a patient that has previously received one or more doses of a VEGF antagonist B (e.g., aflibercept, ranibizumab) or in a patient pretreated with one or more doses of a VEGF antagonist B (e.g., aflibercept or ranibizumab), wherein the method comprises:
(i) administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment; or
(ii) administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease,
   and in particular wherein said method provides decreasing or reducing IRC and/or IRF and/or SRF after the last administration of the VEGF antagonist A (e.g., brolucizumab) compared to a baseline IRC and/or IRF and/or SRF, wherein the baseline IRC and/or IRF and/or SRF was assessed prior to the first administration of the VEGF antagonist B (e.g., aflibercept or ranibizumab).

In one aspect, the present disclosure provides methods of inhibiting VEGF or suppression of VEGF levels in retina of a patient that has previously received one or more doses of a VEGF antagonist B (e.g., aflibercept, ranibizumab) or in a patient pretreated with one or more doses of a VEGF antagonist B (e.g., aflibercept or ranibizumab), wherein the method comprises:
(i) administering to the patient an initial dose of a VEGF antagonist A (e.g., brolucizumab) followed by one or more additional doses of the VEGF antagonist A, in particular wherein said one or more additional doses of the VEGF antagonist A are administered in an administration interval of no less than about 8 weeks and/or as individualized by a physician based on a disease activity assessment; or
(ii) administering to the patient an initial dose of a VEGF antagonist A (e.g., brolucizumab) followed by one or more additional doses of the VEGF antagonist A in an administration interval according to a maintenance regimen of the VEGF antagonist A for the treatment of the ocular disease,
   and in particular wherein the patient treated with the methods of the present disclosure has retinal levels of free-VEGF concentration below a target threshold of 10 pM, preferably below a target threshold of 5 pM, over at least 85% (e.g., at least 90%, at least 95%, preferably 100%) of the duration of the treatment period, in particular wherein the treatment period is at least 6 months, preferably at least 12 months or 52 weeks.

In a further aspect, the disclosure invention provides a VEGF antagonist A (e.g., brolucizumab) for use as a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the VEGF antagonist A (e.g., brolucizumab) is administered to the patient as an initial dose followed by one or more additional doses, in particular wherein said one or more additional doses of the VEGF antagonist A are administered in an administration interval of no less than about 8 weeks and/or as individualized by a physician based on a disease activity assessment. Suitably, the disclosure invention provides a Compound A (e.g., brolucizumab) for use as a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), wherein the Compound A (e.g., brolucizumab) is administered to the patient as an initial dose followed by one or more additional doses, in particular wherein said one or more additional doses of the Compound A are administered in an administration interval of no less than about 8 weeks and/or as individualized by a physician based on a disease activity assessment, wherein the Compound A and the Compound B are VEGF antagonists, and wherein the Compound A is different from the Compound B.

In a further aspect, the present disclosure provides a VEGF antagonist A (e.g., brolucizumab) for use as a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the VEGF antagonist A (e.g., brolucizumab) is administered to the patient as an initial dose followed by one or more additional doses in an administration interval according to a maintenance regimen of the VEGF antagonist A for the treatment of the ocular disease. Suitably, the present disclosure provides a Compound A (e.g., brolucizumab) for use as a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), wherein the Compound A (e.g., brolucizumab) is administered to the patient as an initial dose followed by one or more additional doses in an administration interval according to a maintenance regimen of the Compound A for the treatment of the ocular disease, wherein the Compound A and the Compound B are VEGF antagonists, and wherein the Compound A is different from the Compound B.

In a further aspect, the present disclosure provides a pharmaceutical composition comprising a VEGF antagonist A for use as a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the pharmaceutical composition comprising a VEGF antagonist A (e.g., brolucizumab) is administered to the patient as an initial dose followed by one or more additional doses, in particular wherein said one or more additional doses of the VEGF antagonist A are administered in an administration interval of no less than about 8 weeks and/or as individualized by a physician based on a disease activity assessment. Suitably, the present disclosure provides a pharmaceutical composition comprising a Compound A for use as a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), wherein the pharmaceutical composition comprising a Compound A (e.g., brolucizumab) is administered to the patient as an initial dose followed by one or more additional doses, in particular wherein said one or more additional doses of the Compound A are administered in an administration interval of no less than about 8 weeks and/or as individualized by a physician based on a disease activity assessment, and wherein the Compound A and the Compound B are VEGF antagonists, and wherein the Compound A is different from the Compound B.

In a further aspect, the present disclosure provides a pharmaceutical composition comprising a VEGF antagonist A for use as a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), wherein the pharmaceutical composition comprising a VEGF antagonist A (e.g., brolucizumab) is administered to the patient as an initial dose followed by one or more additional doses in an administration interval according to a maintenance regimen of the VEGF antagonist A for the treatment of the ocular disease. Suitably, the present disclosure provides a pharmaceutical composition comprising a Compound A for use as a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), wherein the pharmaceutical composition comprising a Compound A (e.g., brolucizumab) is administered to the patient as an initial dose followed by one or more additional doses in an administration interval according to a maintenance regimen of the Compound A for the treatment of the ocular disease, and wherein the Compound A and the Compound B are VEGF antagonists, and wherein the Compound A is different from the Compound B.

In one aspect, the present disclosure provides use of a VEGF antagonist A (e.g., brolucizumab) for the manufacture of a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A, in particular wherein said one or more additional doses of the VEGF antagonist A are administered in an administration interval of no less than about 8 weeks and/or as individualized by a physician based on a disease activity assessment. Suitably, the present disclosure provides use of a Compound A (e.g., brolucizumab) for the manufacture of a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), the use comprises administering to the patient an initial dose of a Compound A (e.g., brolucizumab) followed by one or more additional doses of a Compound A, in particular wherein said one or more additional doses of the Compound A are administered in an administration interval of no less than about 8 weeks and/or as individualized by a physician based on a disease activity assessment, and wherein the Compound A and the Compound B are VEGF antagonists, and wherein the Compound A is different from the Compound B.

In one aspect, the present disclosure provides use of a VEGF antagonist A (e.g., brolucizumab) for the manufacture of a medicament for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A in an administration interval according to a maintenance regimen of the VEGF antagonist A for the treatment of the ocular disease. Suitably, the present disclosure provides use of a Compound A (e.g., brolucizumab) for the manufacture of a medicament for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), the use comprises administering to the patient an initial dose of a Compound A (e.g., brolucizumab) followed by one or more additional doses of a Compound A in an administration interval according to a maintenance regimen of the Compound A for the treatment of the ocular disease, wherein the Compound A and the Compound B are VEGF antagonists, and wherein the Compound A is different from the Compound B.

In one aspect, the present disclosure provides use of a VEGF antagonist A (e.g., brolucizumab) for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprises administering to the patient an initial dose of a VEGF antagonist A (e.g., brolucizumab) followed by one or more additional doses of a VEGF antagonist A, in particular wherein said one or more additional doses of the VEGF antagonist A are administered in an administration interval of no less than about 8 weeks and/or as individualized by a physician based on a disease activity assessment. Suitably, the present disclosure provides use of a Compound A (e.g., brolucizumab) for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), the use comprises administering to the patient an initial dose of a Compound A (e.g., brolucizumab) followed by one or more additional doses of a Compound A, in particular wherein said one or more additional doses of the Compound A are administered in an administration interval of no less than about 8 weeks and/or as individualized by a physician based on a disease activity assessment, and wherein the Compound A and the Compound B are VEGF antagonists, and wherein the Compound A is different from the Compound B.

In one aspect, the present disclosure provides use of a VEGF antagonist A (e.g., brolucizumab) for treating ocular disease, in particular neovascular age-related macular degeneration (nAMD), in a patient that has previously received one or more doses of a VEGF antagonist B (e.g., aflibercept or ranibizumab) or in a patient pretreated with one or more doses of a VEGF antagonist B (e.g., aflibercept or ranibizumab), the use comprising administering to the patient an initial dose of a VEGF antagonist A (e.g., brolucizumab) followed by one or more additional doses of a VEGF antagonist A in an administration interval according to a maintenance regimen of the VEGF antagonist A for the treatment of the ocular disease. Suitably, the present disclosure provides use of a Compound A (e.g., brolucizumab) for treating ocular disease, in particular neovascular age-related macular degeneration (nAMD), in a patient that has previously received one or more doses of a Compound B (e.g., aflibercept or ranibizumab) or in a patient pretreated with one or more doses of a Compound B (e.g., aflibercept or ranibizumab), the use comprising administering to the patient an initial dose of a Compound A (e.g., brolucizumab) followed by one or more additional doses of a Compound A in an administration interval according to a maintenance regimen of the Compound A for the treatment of the ocular disease, wherein the Compound A and the Compound B are VEGF antagonists, and wherein the Compound A is different from the Compound B.

In one embodiment, the ocular disease is selected from the list consisting of abnormal angiogenesis, choroidal neovascularization (CNV), choroidal neovascularization (CNV) associated with nAMD, retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization. In one embodiment, the disease is diabetic macular edema (DME). In one embodiment, the disease is diabetic retinopathy (DR). In a one embodiment, the disease is proliferative diabetic retinopathy (PDR). In a one embodiment, the disease is non-proliferative diabetic retinopathy (NPDR). In one embodiment, the disease is Retinal Vein Occlusion (RVO), e.g., Central Retinal Vein Occlusion (CRVO), e.g., Branch Retinal Vein Occlusion (BRVO). In a preferred embodiment, the disease is nAMD. In another preferred embodiment, the disease is CNV associated with nAMD.

The methods and uses of the present disclosure are suitable for a patient that has previously received one or more doses of a Compound B or VEGF antagonist B (e.g., aflibercept or ranibizumab) or in a patient pretreated with one or more doses of a Compound B or VEGF antagonist B (e.g., aflibercept or ranibizumab) or a patient switched from a therapy with a Compound B or VEGF antagonist B (e.g., aflibercept or ranibizumab) to a therapy with a Compound A or VEGF antagonist A (e.g., brolucizumab).

In certain embodiments, the patient was pretreated with one or more doses of a VEGF antagonist B (Compound B), e.g., any licensed anti-VEGF drug, e.g., aflibercept or ranibizumab. In certain embodiments, the patient was pretreated with one or more doses of a VEGF antagonist B (Compound B), e.g., any licensed anti-VEGF drug, e.g., aflibercept or ranibizumab, for at least 3 months or longer, preferably for at least 6 months or longer. In certain embodiments, the VEGF antagonist B (Compound B), e.g., aflibercept or ranibizumab, was administered to the patient in an administration interval, e.g., an injection interval, of about every 4 weeks or longer (≥q4w), e.g., between about ≥4 and about ≤24 weeks (≥q4w to ≤q24w), between about ≥4 and about ≤18 weeks (≥q4w to ≤q18w), between about ≥4 and about ≤12 weeks (≥q4w to ≤q12w), between about ≥4 and about ≤10 weeks (≥q4w to ≤q10w), in particular between about ≥6 and about ≤10 weeks (≥q6w to ≤q10w), in more particular between about ≥6 and about ≤8 weeks (≥q6w to ≤q8w), e.g., between 4 and ≤24 weeks (≥q4w to ≤q24w), between 4 and ≤18 weeks (≥q4w to ≤q18w), between 4 and ≤12 weeks (≥q4w to ≤q12w), between ≥4 and ≤10 weeks (≥q4w to ≤q0w), in particular between ≥6 and ≤10 weeks (≥q6w to ≤q0w), in more particular between 6 and ≤8 weeks (≥q6w to ≤q8w). In certain embodiments, the VEGF antagonist B (Compound B), e.g., aflibercept or ranibizumab, was administered to the patient in an administration interval, e.g., an injection interval, of about every month or longer, e.g., between ≥one month and ≤6 months, between ≥one month and ≤5 months, between ≥one month and ≤4 months, between ≥one month and ≤3 months, between ≥one month and ≤2 months, in particular ≤2 months administration interval.

In some embodiments, the patient has had an inadequate response or did not respond adequately or failed to respond to a treatment with the VEGF antagonist B (Compound B), e.g., any licensed anti-VEGF drug, e.g., aflibercept or ranibizumab. In more specific embodiments, the patient has had an inadequate response or did not respond adequately or failed to respond to a maintenance treatment with the VEGF antagonist B (Compound B), e.g., any licensed anti-VEGF drug, e.g., aflibercept or ranibizumab.

The term “inadequate response”, as used herein, means that the patient treated with one or more doses of a VEGF antagonist B (Compound B), e.g., a patient treated with the VEGF antagonist B (Compound B), e.g., aflibercept or ranibizumab, for at least about 3 months or longer, preferably for at least about 6 months or longer) has no significant improvement (insufficient response) in the ocular disease treated, e.g., nAMD, or has a suboptimal anatomically controlled ocular disease, e.g., nAMD, or has presence of ocular disease activity, e.g., nAMD disease activity.

In one example, no significant improvement or insufficient response in the ocular disease treated, e.g., nAMD, is characterized by one or more of the following: (i) no improvement or no significant improvement or only little improvement in best corrected visual acuity (BCVA); (ii) no improvement or no significant improvement or only little improvement in visual acuity (VA); (iii) no improvement or no significant improvement or only little improvement in central subfield thickness (CSFT); and/or (iv) no improvement or no significant improvement or only little improvement in intraretinal cysts/fluid. In one example, no significant improvement (insufficient response) in the ocular disease treated, e.g., nAMD, is characterized by one or more of the following:
(i) no increase or increase of no more than 2 or 3 letters for BCVA after 3 months or after 6 months or after 9 months or longer of treatment with the VEGF antagonist B (Compound B);
(ii) no increase or increase of no more than 1 or 2 letters for visual acuity (VA) after 3 months or after 6 months of treatment with the VEGF antagonist B (Compound B);
(iii) no reduction or decrease of no more than ≥25 μm, e.g., ≥50 μm, in central subfield thickness (CSFT) after 3 months or after 6 months or after 9 months or longer of treatment with the VEGF antagonist B (Compound B);
(iv) presence of residual or recurrent intraretinal cysts/fluid after 3 months or after 6 months or after 9 months or longer of treatment with the VEGF antagonist B (Compound B).

In one embodiment, the patient pretreated with one or more doses of the VEGF antagonist B (Compound B), e.g., aflibercept or ranibizumab, has no significant improvement or has an insufficient response in the ocular disease treated, e.g., nAMD (i.e. residual or recurrent fluid indicating disease activity, in particular residual or recurrent fluid indicating disease activity in a ≥q6w to ≤q10w administration interval, in more particular in a ≥q6w to ≤q8w administration interval).

In certain embodiments, the patient treated with one or more doses of the VEGF antagonist B (Compound B) has suboptimal anatomically controlled ocular disease, e.g., nAMD. In one example, suboptimal anatomically controlled ocular disease, e.g., nAMD, is characterized by one or more of the following:
(i) no reduction or decrease of no more than ≥25 μm, e.g., ≥50 μm, in central subfield thickness (CSFT) after 3 months or after 6 months or after 9 months or longer of treatment with the VEGF antagonist B (Compound B);
(ii) presence of residual or recurrent intraretinal cysts/fluid after 3 months or after 6 months or after 9 months or longer of treatment with the VEGF antagonist B (Compound B).

In certain embodiments, presence of ocular disease activity, e.g., nAMD disease activity was identified in the patient treated with one or more doses of the VEGF antagonist B (Compound B). The presence of an ocular disease, in particular nAMD, may include one or more of the following: (i) decrease in Best Corrected Visual Acuity (BCVA), (ii) decrease in Visual Acuity (VA), (iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF). Suitably, the presence of an ocular disease, in particular nAMD, may include one or more of the following:
(i) decrease in BCVA of ≥2 letters, e.g., decrease in BCVA of ≥3 letters, decrease in BCVA of ≥4 letters, decrease in BCVA of ≥5 letters, in particular wherein:
   the decrease in BCVA is observed at Week 4 or at Week 6 or at Week 8 or Week 12 after the last administration of the VEGF antagonist B compared to a baseline BCVA, wherein the baseline BCVA was assessed prior to the last administration of the VEGF antagonist B (Compound B), or
   the decrease in BCVA is observed after 4 months or longer, e.g., after 6 months or longer, of the administration of the VEGF antagonist B compared to a baseline BCVA, wherein the baseline BCVA was assessed 4 months or longer, e.g., 6 months or longer, prior to the last administration of the VEGF antagonist B (Compound B);
(ii) decrease in VA of ≥1 letters, e.g., decrease in VA of ≥2 letters, decrease in VA of ≥3 letters, in particular wherein:
   the decrease in VA is observed at Week 4 or at Week 6 or at Week 8 or Week 12 after the last administration of the VEGF antagonist B compared to a baseline VA, wherein the baseline VA was assessed prior to the last administration of the VEGF antagonist B (Compound B), or
   the decrease in VA is observed after 4 months or longer, e.g., after 6 months or longer, of the administration of the VEGF antagonist B compared to a baseline VA, wherein the baseline VA was assessed 4 months or longer, e.g., 6 months or longer, prior to the last administration of the VEGF antagonist B (Compound B);
(iii) CSFT increase ≥25 μm, e.g., CSFT increase ≥50 μm, CSFT increase ≥75 μm, in particular wherein:
   the CSFT increase is observed at Week 4 or at Week 6 or at Week 8 or Week 12 after the last administration of the VEGF antagonist B compared to a baseline CSFT, wherein the baseline CSFT was assessed prior to the last administration of the VEGF antagonist B (Compound B), or
   the CSFT increase is observed after 4 months or longer, e.g., after 6 months or longer, of the administration of the VEGF antagonist B compared to a baseline CSFT, wherein the baseline CSFT was assessed 4 months or longer, e.g., 6 months or longer, prior to the last administration of the VEGF antagonist B (Compound B);
(iv) new or persistent or recurrent intraretinal cysts (IRC) and/or intraretinal fluid (IRF) and/or subretinal fluid (SRF), in particular wherein:
   new or persistent or recurrent intraretinal cysts (IRC) and/or intraretinal fluid (IRF) and/or subretinal fluid (SRF) are observed at Week 4 or at Week 6 or at Week 8 or Week 12 after the last administration of the VEGF antagonist B compared to a baseline IRC and/or IRF and/or SRF, wherein the baseline IRC and/or IRF and/or SRF was assessed prior to the last administration of the VEGF antagonist B (Compound B), or
   new or persistent or recurrent intraretinal cysts (IRC) and/or intraretinal fluid (IRF) and/or subretinal fluid (SRF) are observed after 4 months or longer, e.g., after 6 months or longer, of the administration of the VEGF antagonist B (Compound B) compared to a baseline IRC and/or IRF and/or SRF, wherein the baseline IRC and/or IRF and/or SRF was assessed 4 months or longer, e.g., 6 months or longer, prior to the last administration of the VEGF antagonist B (Compound B).

In certain embodiments, the patient is at least 18 years of age, e.g., at least 50 years of age, e.g., at least 65 years of age, and has suboptimal anatomically controlled nAMD (i.e. residual or recurrent fluid indicating disease activity, in particular residual or recurrent fluid indicating disease activity in a q4w to ≤q10w injection interval, more particularly residual or recurrent fluid indicating disease activity in a q6w to ≤q10w injection interval).

In certain embodiments, the patient has been diagnosed with active choroidal neovascularization (CNV) secondary to AMD, and treated previously for this disease. In one embodiment, the pretreated patient has one or more, or all, of the following:
active choroidal neovascularization (CNV) secondary to AMD that affects the central subfield, including retinal angiomatous proliferation (RAP) with a CNV component, confirmed by presence of active leakage from CNV seen by fluorescein angiography and sequelae of CNV, e.g. pigment epithelial detachment (PED), subretinal or sub-retinal pigment epithelium (sub-RPE) hemorrhage, blocked fluorescence, macular edema (intraretinal fluid (IRF) and/or subretinal fluid (SRF) and/or sub-retinal pigment epithelium (sub-RPE) fluid that affects the central subfield, as seen by SD-OCT) as confirmed by central reading center;
previous treatment with any licensed anti-VEGF drug for ≥6 months in a ≥q6w to ≤q10w administration interval (e.g., injection interval) with residual or recurrent fluid (intraretinal fluid (IRF) and/or subretinal fluid (SRF)) that affects the central subfield, as seen by SD-OCT), despite continuous licensed anti-VEGF treatment at baseline. In total, patients should not be pretreated for more than 36 months; and
BCVA score must be ≤83 and ≥38 letters at 4 meters starting distance using Early Treatment Diabetic Retinopathy Study (ETDRS)-like visual acuity charts (approximately Snellen equivalent of 20/25 and 20/200), at both screening and baseline.

In some embodiments, the patient treated with one or more doses of the VEGF antagonist B (e.g., aflibercept or ranibizumab) was intolerant to the treatment with the VEGF antagonist B.

The term “intolerant”, as used herein, refers to a patient having one or more side effects in response to the treatment, in particular adverse side effects, e.g., any unfavorable and unintended sign (including abnormal laboratory findings), or symptom or disease in a subject. Suitably, one or more side effects in response to the treatment with the VEGF antagonist B (Compound B) may include hypersensitivity to the VEGF antagonist B (Compound B), blurred vision, cataract, conjunctival hemorrhage, vitreous floaters, eye pain, intraocular inflammation, intraocular pressure increased, retinal hemorrhage, vitreous detachment, conjunctivitis, retinal pigment epithelial tear, corneal abrasion, hypersensitivity, punctate keratitis, retinal tear, endophthalmitis, blindness, retinal artery occlusion, retinal detachment, conjunctival hyperemia, lacrimation increased, abnormal sensation in eye, detachment of retinal pigment epithelium, retinal vasculitis and/or retinal vascular occlusion, e.g., retinal vasculitis and/or retinal vascular occlusion in the presence of intraocular inflammation. In certain embodiments, the patient pretreated with one or more doses of the VEGF antagonist B (Compound B) was hypersensitive or developed hypersensitivity to the VEGF antagonist B (Compound B) or to any of the excipients in a pharmaceutical composition comprising the VEGF antagonist B (Compound B). Hypersensitivity reactions may manifest as rash, pruritus, urticaria, erythema, or severe intraocular inflammation.

In some embodiments, the methods and uses of the present disclosure comprise administering to the patient one or more doses of the VEGF antagonist A (Compound A), e.g., brolucizumab, wherein the patient does not have (i) ocular inflammation, in particular active ocular inflammation, and/or (ii) retinal vasculitis and/or retinal vascular occlusion, e.g., retinal vasculitis and/or retinal vascular occlusion in the presence of intraocular inflammation.

In one embodiment, the methods and uses of the present disclosure comprise discontinuing treatment with the VEGF antagonist B (Compound B) and instead administering to the patient one or more doses of the VEGF antagonist A (Compound A), e.g., brolucizumab. In one embodiment, the methods and uses of the present disclosure comprise administering to the patient one or more doses of the VEGF antagonist A (Compound A), e.g., brolucizumab, and wherein the VEGF antagonist A (Compound A) is administered in replacement of the VEGF antagonist B (Compound B) and no additional or alternative VEGF antagonists are administered to the patient during administration of the VEGF antagonist A (Compound A).

Suitably, the methods and the uses of the disclosure comprise administering to the patient one or more doses of the VEGF antagonist A (Compound A), e.g., brolucizumab, wherein the initial dose of the VEGF antagonist A (Compound A) is administered to the patient according to the dosing schedule for the VEGF antagonist B (Compound B), e.g., aflibercept or ranibizumab.

Suitably, the methods and the uses of the disclosure comprise administering to the patient one or more doses of the VEGF antagonist A (Compound A), e.g., brolucizumab, wherein the initial dose of the VEGF antagonist A is administered to the patient up to about 24 weeks or shorter, e.g., up to about 21 weeks or shorter, up to about 18 weeks or shorter, up to about 16 weeks or shorter, up to about 12 weeks or shorter, in particular up to about 10 weeks or shorter, more particularly up to about 7 weeks or shorter, e.g., up to about 6 weeks or shorter, up to about 5 weeks or shorter, up to about 4 weeks or shorter, after the last dose of the VEGF antagonist B (Compound B), e.g., aflibercept or ranibizumab, was administered to the patient.

In some embodiments, the period of time between discontinuing treatment with the VEGF antagonist B (e.g., aflibercept or ranibizumab) and start of administration of the VEGF antagonist A (e.g., brolucizumab) is up to about 24 weeks or shorter, e.g., up to about 21 weeks or shorter, up to about 18 weeks or shorter, up to about 16 weeks or shorter, up to about 12 weeks or shorter, in particular up to about 10 weeks or shorter, more particularly up to about 7 weeks or shorter, e.g., up to about 6 weeks or shorter, up to about 5 weeks or shorter, up to about 4 weeks or shorter. In some embodiments, the period of time between discontinuing treatment with the VEGF antagonist B (e.g., aflibercept or ranibizumab) and start of the administration of the VEGF antagonist A (e.g., brolucizumab) is up to about one month, or up to about two months, or up to about three months, or up to about four months, or up to about five months, or up to about six months. Suitably, the methods and the uses of the disclosure comprise administering to the patient one or more doses of the VEGF antagonist A (e.g., brolucizumab), wherein the initial dose of the VEGF antagonist A is administered to the patient up to about three months, in particular up to about two months, after the last dose of the VEGF antagonist B (e.g., aflibercept or ranibizumab) was administered to the patient.

In a specific embodiment, the methods and the uses of the disclosure comprise administering to the patient one or more doses of the VEGF antagonist A (Compound A), e.g., brolucizumab, wherein the initial dose of the VEGF antagonist A is administered to the patient up to about 14 weeks, up to about 12 weeks, up to about 10 weeks, in particular up to 8 weeks, more particularly up to about 7 weeks, after the last dose of the VEGF antagonist B (Compound B) was administered to the patient, wherein the VEGF antagonist B is ranibizumab. In a specific embodiment, the period of time between discontinuing treatment with the VEGF antagonist B and start of administration of the VEGF antagonist A (e.g., brolucizumab) is up to about 14 weeks, up to about 12 weeks, up to about 10 weeks, in particular up to 8 weeks, more particularly up to about 7 weeks, wherein the VEGF antagonist B is ranibizumab. In a specific embodiment, the period of time between discontinuing treatment with the VEGF antagonist B and start of the administration of the VEGF antagonist A (e.g., brolucizumab) is up to about two months, or up to about three months, wherein the VEGF antagonist B is ranibizumab.

In a specific embodiment, the methods and the uses of the disclosure comprise administering to the patient one or more doses of the VEGF antagonist A (Compound A), e.g., brolucizumab, wherein the initial dose of the VEGF antagonist A is administered to the patient up to about 24 weeks, e.g., up to about 21 weeks, up to about 18 weeks, in particular up to about 16 weeks, more particularly up to about 12 weeks, after the last dose of the VEGF antagonist B (Compound B) was administered to the patient, wherein the VEGF antagonist B is aflibercept. In a specific embodiment, the period of time between discontinuing treatment with the VEGF antagonist B and start of administration of the VEGF antagonist A (e.g., brolucizumab) is up to about 24 weeks, e.g., up to about 21 weeks, up to about 18 weeks, in particular up to about 16 weeks, more particularly up to about 12 weeks, wherein the VEGF antagonist B is aflibercept. In a specific embodiment, the period of time between discontinuing treatment with the VEGF antagonist B and start of the administration of the VEGF antagonist A (e.g., brolucizumab) is up to about three months, or up to about four months, or up to about five months, wherein the VEGF antagonist B is aflibercept.

In a specific embodiment, the methods and the uses of the disclosure comprise administering to the patient one or more doses of the VEGF antagonist A (Compound A), e.g., aflibercept or ranibizumab, wherein the initial dose of the VEGF antagonist A is administered to the patient up to about 14 weeks, e.g., up to about 13 weeks, up to about 12 weeks, up to about 11 weeks, in particular up to about 10 weeks, after the last dose of the VEGF antagonist B was administered to the patient, wherein the VEGF antagonist B (Compound B) is brolucizumab. In a specific embodiment, the period of time between discontinuing treatment with the VEGF antagonist B and start of administration of the VEGF antagonist A (e.g., aflibercept or ranibizumab) is up to about 14 weeks, e.g., up to about 13 weeks, up to about 12 weeks, up to about 11 weeks, in particular up to about 10 weeks, wherein the VEGF antagonist B is brolucizumab. In a specific embodiment, the period of time between discontinuing treatment with the VEGF antagonist B and start of the administration of the VEGF antagonist A e.g., aflibercept or ranibizumab) is up to about two months, or up to about three months, or up to about four months, wherein the VEGF antagonist B is brolucizumab.

Suitably, the methods and the uses of the disclosure comprise administering to the patient one or more doses of the VEGF antagonist A (Compound A), e.g., brolucizumab, wherein the initial dose of the VEGF antagonist A is administered to the patient between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B (Compound B), e.g., aflibercept or ranibizumab, was administered to the patient. In some embodiments, the period of time between discontinuing treatment with the VEGF antagonist B (e.g., aflibercept or ranibizumab) and start of administration of the VEGF antagonist A (e.g., brolucizumab) is about 4 to 6 weeks, about 4 to 8 weeks, or about 4 to 10 weeks, or about 4 to 12 weeks, or about 4 to 16 weeks, or about 4 to 18 weeks, or about 4 to 21 weeks, or about 4 to 24 weeks, in particular about 6 to 8 weeks, or about 6 to 10 weeks, or about 6 to 12 weeks, or about 6 to 16 weeks, or about 6 to 18 weeks, or about 6 to 21 weeks, or about 6 to 24 weeks. In some embodiments, the period of time between discontinuing treatment with the VEGF antagonist B (e.g., aflibercept or ranibizumab) and start of the administration of the VEGF antagonist A (e.g., brolucizumab) is about one month to about two months, or about one month to about three months, or about one month to about four months, or about one month to about five months, or about one month to about six months. Suitably, the methods and the uses of the disclosure comprise administering to the patient one or more doses of the VEGF antagonist A (e.g., brolucizumab), wherein the initial dose of the VEGF antagonist A is administered to the patient between about one month to about three months, in particular between about one month to about two months, after the last dose of the VEGF antagonist B (e.g., aflibercept or ranibizumab) was administered to the patient.

In a specific embodiment, the methods and the uses of the disclosure comprise administering to the patient one or more doses of the VEGF antagonist A (Compound A), e.g., brolucizumab, wherein the initial dose of the VEGF antagonist A is administered to the patient between about ≥4 and about ≤14 weeks, e.g., about ≥4 and about ≤12 weeks, about ≥4 and about ≤10 weeks, in particular between about ≥4 and about ≤8 weeks, more particularly between about ≥6 and about ≤8 weeks, after the last dose of the VEGF antagonist B (Compound B) was administered to the patient, wherein the VEGF antagonist B is ranibizumab. In a specific embodiment, the period of time between discontinuing treatment with the VEGF antagonist B and start of administration of the VEGF antagonist A (e.g., brolucizumab) is about 4 to 14 weeks, about 4 to 12 weeks, or about 4 to 10 weeks, or about 4 to 8 weeks, or about 6 to 8 weeks, wherein the VEGF antagonist B is ranibizumab. In a specific embodiment, the period of time between discontinuing treatment with the VEGF antagonist B and start of the administration of the VEGF antagonist A (e.g., brolucizumab) is about one month to about three months, or about one month to about two months, wherein the VEGF antagonist B is ranibizumab. Suitably, the methods and the uses of the disclosure comprise administering to the patient one or more doses of the VEGF antagonist A (e.g., brolucizumab), wherein the initial dose of the VEGF antagonist A is administered to the patient between about one month to about three months, in particular between about one month to about two months, after the last dose of the VEGF antagonist B was administered to the patient, wherein the VEGF antagonist B is ranibizumab.

In a specific embodiment, the methods and the uses of the disclosure comprise administering to the patient one or more doses of the VEGF antagonist A (Compound A), e.g., brolucizumab, wherein the initial dose of the VEGF antagonist A is administered to the patient between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤14 weeks (e.g., between about one month and 3 months), in particular between about ≥6 and about ≤14 weeks, more particularly between about ≥8 and about ≤12 weeks, after the last dose of the VEGF antagonist B (Compound B) was administered to the patient, wherein the VEGF antagonist B is aflibercept. In a specific embodiment, the period of time between discontinuing treatment with the VEGF antagonist B and start of administration of the VEGF antagonist A (e.g., brolucizumab) is about 6 to 12 weeks, or about 8 to 12 weeks, or about 8 to 16 weeks, or about 8 to 18 weeks, or about 8 to 21 weeks, or about 8 to 24 weeks, wherein the VEGF antagonist B is aflibercept. In a specific embodiment, the period of time between discontinuing treatment with the VEGF antagonist B and start of the administration of the VEGF antagonist A (e.g., brolucizumab) is about two month to about three months, or about two month to about four months, or about two month to about five months, or about two month to about six months, wherein the VEGF antagonist B is aflibercept. Suitably, the methods and the uses of the disclosure comprise administering to the patient one or more doses of the VEGF antagonist A (e.g., brolucizumab), wherein the initial dose of the VEGF antagonist A is administered to the patient between about two month to about three months, after the last dose of the VEGF antagonist B was administered to the patient, wherein the VEGF antagonist B is aflibercept.

In a specific embodiment, the methods and the uses of the disclosure comprise administering to the patient one or more doses of the VEGF antagonist A (Compound A), e.g., aflibercept or ranibizumab, wherein the initial dose of the VEGF antagonist A is administered to the patient between about ≥4 and about ≤14 weeks, e.g., about ≥4 and about ≤12 weeks (e.g., between about one month and three months), about ≥4 and about ≤10 weeks, in particular between about ≥6 and about ≤10 weeks, more particularly between about ≥8 and about ≤10 weeks, after the last dose of the VEGF antagonist B (Compound B) was administered to the patient, wherein the VEGF antagonist B is brolucizumab. In a specific embodiment, the period of time between discontinuing treatment with the VEGF antagonist B and start of administration of the VEGF antagonist A (e.g., aflibercept or ranibizumab) is between about ≥4 and about ≤14 weeks, e.g., about ≥4 and about ≤12 weeks (e.g., between about one month and three months), about ≥4 and about ≤10 weeks, in particular between about ≥6 and about ≤10 weeks, more particularly between about ≥8 and about ≤10 weeks, wherein the VEGF antagonist B is brolucizumab.

In a specific embodiment, the period of time between discontinuing treatment with the VEGF antagonist B (Compound B) and start of administration of the VEGF antagonist A(Compound A), e.g., aflibercept or ranibizumab, is about 6 to 14 weeks, about 6 to 12 weeks, or about 6 to 10 weeks, or about 8 to 10 weeks, wherein the VEGF antagonist B is brolucizumab. In a specific embodiment, the period of time between discontinuing treatment with the VEGF antagonist B and start of the administration of the VEGF antagonist A (e.g., aflibercept or ranibizumab) is about two month to about three months, wherein the VEGF antagonist B is brolucizumab. Suitably, the methods and the uses of the disclosure comprise administering to the patient one or more doses of the VEGF antagonist A (e.g., aflibercept or ranibizumab), wherein the initial dose of the VEGF antagonist A is administered to the patient between about two month to about three months, after the last dose of the VEGF antagonist B was administered to the patient, wherein the VEGF antagonist B is brolucizumab.

In certain embodiments, the initial dose of the VEGF antagonist A (Compound A), e.g., brolucizumab, is followed by one or more doses of the VEGF antagonist A in an administration interval, e.g., an injection interval, as individualized by a physician based on a disease activity assessment. In certain embodiments, the initial dose of the VEGF antagonist A (e.g., brolucizumab) is followed by one or more doses of the VEGF antagonist A in an administration interval, e.g., an injection interval, of no less than about 4 weeks, preferably no less than about 6 weeks, more preferably no less than about 8 weeks, e.g., no less than about 12 weeks. Suitably, the initial dose of the VEGF antagonist A (e.g., brolucizumab) is followed by one or more doses of the VEGF antagonist A in an administration interval, e.g., an injection interval, between about ≥4 and about ≤24 weeks (≥q4w to ≤q24w), preferably between about ≥6 and about ≤24 weeks (≥q6w to ≤q24w), more preferably between about ≥8 and about ≤24 weeks (≥q8w to ≤q24w), e.g., between about ≥8 and about ≤18 weeks (≥q8w to ≤q18w), between about ≥8 and about ≤12 weeks (≥q8w to ≤q12w). In certain embodiments, the initial dose of the VEGF antagonist A (e.g., brolucizumab) is followed by one or more doses of the VEGF antagonist A in an administration interval, e.g., an injection interval, of no less than about one month, e.g., no less than about two months, no less than about three months, no less than about four months, no less than about five months, no less than about six months. In a preferred embodiment, the initial dose of the VEGF antagonist A (e.g., brolucizumab) is followed by one or more doses of the VEGF antagonist A in an administration interval, e.g., an injection interval, of no less than about two months. In a more preferred embodiment, the initial dose of the VEGF antagonist A (e.g., brolucizumab) is followed by one or more doses of the VEGF antagonist A in an administration interval, e.g., an injection interval, of no less than about three months. Suitably, the initial dose of the VEGF antagonist A (e.g., brolucizumab) is followed by one or more doses of the VEGF antagonist A in an administration interval, e.g., an injection interval, between about one month and about 6 months, preferably between about 2 months and about 6 months (e.g., about 2 months and about 3 months), more preferably between about 3 months and about 6 months.

Suitably, the methods and the uses of the disclosure comprise administering to the patient pretreated with another VEGF therapy an initial administration, e.g., injection, of the VEGF antagonist A (Compound A), e.g., brolucizumab, followed by subsequent administrations, e.g., injections with the VEGF antagonist A (e.g., brolucizumab) once every 8 weeks (2 months) to 12 weeks (3 months) and/or as individualized by a physician based on a disease activity assessment, in particular once every 12 weeks (3 months). Suitably, the methods and the uses of the disclosure comprise administering to the patient who failed to respond to treatment with another VEGF therapy an initial administration, e.g., injection, of the VEGF antagonist A (e.g., brolucizumab), followed by subsequent administrations, e.g., injections, with the VEGF antagonist A (e.g., brolucizumab) once every 8 weeks (2 months) to 12 weeks (3 months) and/or as individualized by a physician based on a disease activity assessment, in particular once every 12 weeks (3 months).

Suitably, the disclosure provides the methods and the uses, wherein when starting the patient on the VEGF antagonist A (Compound A), e.g., brolucizumab, from another VEGF treatment, e.g., a VEGF antagonist B treatment (Compound B treatment), e.g., aflibercept or ranibizumab, no loading of the VEGF antagonist A (e.g., 3 monthly injections or three q4w injections or three q6w injections) is needed. Suitably, the administration regiment of the VEGF antagonist A of the methods and the uses of the disclosure does not need a loading phase of the VEGF antagonist A (e.g., 3 monthly injections or three q4w injections or three q6w injections). Suitably, patients who failed to respond to a maintenance treatment of another VEGF antagonist, e.g., a VEGF antagonist B (e.g., aflibercept or ranibizumab) are switched to a VEGF antagonist A (e.g., brolucizumab) maintenance regimen. Suitably, the initial dose of the VEGF antagonist A (e.g., brolucizumab) is followed by one or more doses of the VEGF antagonist A with an administration interval according to a maintenance phase regimen of the VEGF antagonist A for the treatment of the ocular disease, e.g., nAMD, in particular as specified on the corresponding drug label for the VEGF antagonist A. Suitably, the initial dose of the VEGF antagonist A (e.g., brolucizumab) is followed by one or more doses of the VEGF antagonist A with an administration interval according to a maintenance phase regimen of the VEGF antagonist A for the treatment of the ocular disease, e.g., nAMD, and without a loading phase of the VEGF antagonist A (e.g., without the first three monthly injections of the VEGF antagonist A).

In some embodiments, the methods and the uses of the disclosure comprise administering to the patient one or more doses of the VEGF antagonist A (Compound A), e.g., brolucizumab, in an administration interval, e.g., an injection interval, as describe herein and without a loading phase of the VEGF antagonist A.

In certain embodiments, the patient pretreated with the VEGF antagonist B (Compound B), e.g., aflibercept or ranibizumab, is switched to the VEGF antagonist A (Compound A), e.g., brolucizumab, and receives an initial injection of the VEGF antagonist A followed by an additional injection about every 12 weeks (q12w). In certain embodiments, the patient pretreated with the VEGF antagonist B (e.g., aflibercept or ranibizumab) is switched to the VEGF antagonist A (e.g., brolucizumab) and receives an initial injection of the VEGF antagonist A followed by an additional injection about every 8 weeks (q8w). The injections after the initial injection of the VEGF antagonist A are referred to here as a “maintenance phase”. The maintenance phase can include administering additional doses at about 4-week, about 5-week, about 6-week, about 7-week, about 8-week, about 9-week, about 10-week, about 11-week, about 12-week, about 13-week, about 14-week, about 15-week, about 16-week, about 17-week, about 18-week, about 19-week, about 20-week, about 21-week, about 22-week, about 23-week, or about 24-week intervals, including a combination of such intervals. In a preferred embodiment, the maintenance phase includes administering additional doses at about 8-week, about 9-week, about 10-week, about 11-week, about 12-week, about 13-week, about 14-week, about 15-week, about 16-week, about 17-week, about 18-week, about 19-week, about 20-week, about 21-week, about 22-week, about 23-week, or about 24-week intervals, including a combination of such intervals. The maintenance phase can include administering additional doses at about one month, about 2 months, about 3 months, about 4 months, about 5 months, about 6 months intervals, including a combination of such intervals. In a preferred embodiment, the maintenance phase includes administering additional doses at about 2 months, about 3 months, about 4 months, about 5 months, about 6 months intervals, including a combination of such intervals.

In one embodiment, the methods and uses of the disclosure comprise discontinuing treatment with the VEGF antagonist B (Compound B) and instead administering to the patient one or more doses of the VEGF antagonist A (Compound A), e.g., brolucizumab, and wherein the methods and the uses comprise administering the VEGF antagonist A to the pretreated patient according to the following schedule: (i) an initial dose the VEGF antagonist A, and (ii) a “maintenance phase” of additional doses of the VEGF antagonist A administered at about 8 to about 12-week intervals, e.g., about 8 week intervals or about 12 week intervals, or at about 2 months to about 3 months intervals, e.g., about 2 months intervals or about 3 months intervals.

The patient is suitable for treatment according to the methods and the uses of the present disclosure independent on the level VEGF production rate within the eye of the patient, e.g., in the retina, vitreous, and aqueous compartments. The patient suitable for treatment according to the methods and the uses of the present disclosure may have a low-level VEGF production rate within the eye, e.g., in the retina, vitreous, and aqueous compartments, in particular wherein the level of VEGF production is ≤10 fmol/day, in particular is ≤5 fmol/day, more particularly is ≤4 fmol/day. The patient suitable for treatment according to the methods and the uses of the present disclosure may have a medium-level (or mean-level) VEGF production rate within the eye, e.g., in the retina, vitreous, and aqueous compartments, in particular wherein the level of VEGF production is >10 fmol/day<25 fmol/day, e.g., >10 fmol/day and ≤20 fmol/day, ≥15 fmol/day and ≤20 fmol/day, in particular is ≥16 fmol/day and ≤18 fmol/day, more particularly about ≥17 fmol/day and ≤18 fmol/day. The patient suitable for treatment according to the methods and the uses of the present disclosure may have a high-level VEGF production rate within the eye, e.g., in the retina, vitreous, and aqueous compartments, in particular wherein the level of VEGF production is ≥25 fmol/day, in particular is ≥30 fmol/day.

In a specific embodiment, the present disclosure provides methods and uses for treating neovascular age-related macular degeneration (nAMD) in a patient that has previously received one or more doses of a VEGF antagonist B (Compound B), e.g., aflibercept or ranibizumab, or in a patient pretreated with one or more doses of a VEGF antagonist B (e.g., aflibercept or ranibizumab), the method or the use comprising administering to the patient the initial dose of a VEGF antagonist A (Compound A), e.g., brolucizumab, followed by one or more doses of the VEGF antagonist A with an administration interval between about 8 and about 12 weeks, in particular wherein the VEGF antagonist A is brolucizumab, and more particularly wherein the dose of the VEGF antagonist A (e.g., the initial dose and the following doses) is from about 3 mg to about 6 mg, in particular about 3 mg or about 6 mg, more particularly 6 mg.

In a more specific embodiment, the present disclosure provides methods and uses for treating neovascular age-related macular degeneration (nAMD) in a patient that has previously received one or more doses of a VEGF antagonist B (Compound B), e.g., aflibercept or ranibizumab, or in a patient pretreated with one or more doses of a VEGF antagonist B (Compound B), e.g., aflibercept or ranibizumab, the method or the use comprising:
(a) administering to the patient the initial dose of a VEGF antagonist A (Compound A), e.g., brolucizumab, between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B (e.g., aflibercept or ranibizumab) was administered to the patient,
(b) followed by administering one or more doses of the VEGF antagonist A with an administration interval between about 8 and about 12 weeks,
   in particular wherein the VEGF antagonist A is brolucizumab, and
   more particularly wherein the dose of the VEGF antagonist A (e.g., the initial dose and the following doses) is from about 3 mg to about 6 mg, in particular about 3 mg or about 6 mg, more particularly 6 mg.

In a more specific embodiment, the present disclosure provides methods and uses for treating neovascular age-related macular degeneration (nAMD) in a patient that has previously received one or more doses of a VEGF antagonist B (Compound A), e.g., aflibercept or ranibizumab, or in a patient pretreated with one or more doses of a VEGF antagonist B (Compound A), e.g., aflibercept or ranibizumab, the method or the use comprising:
(a) discontinuing treatment with VEGF antagonist B, or replacing VEGF antagonist B with a VEGF antagonist A (e.g., brolucizumab)
(b) administering to the patient the initial dose of the VEGF antagonist A (Compound A), e.g., brolucizumab, between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B was administered to the patient;
(c) followed by administering one or more doses of the VEGF antagonist A with an administration interval between about 8 and about 12 weeks,
   in particular wherein the VEGF antagonist A is brolucizumab, and
   more particularly wherein the dose of the VEGF antagonist A (e.g., the initial dose and the following doses) is from about 3 mg to about 6 mg, in particular about 3 mg or about 6 mg, more particularly 6 mg.

In a more specific embodiment, the present disclosure provides methods and uses for treating neovascular age-related macular degeneration (nAMD) in a patient that has previously received one or more doses of a VEGF antagonist B (Compound B) or in a patient pretreated with one or more doses of a VEGF antagonist B (Compound B), the method or the use comprising:
(a) administering to the patient the initial dose of a VEGF antagonist A (Compound A), e.g., aflibercept or ranibizumab, between about ≥4 and about ≤14 weeks, e.g., about ≥4 and about ≤12 weeks (e.g., between about one month and three months), about ≥4 and about ≤10 weeks, in particular between about ≥6 and about ≤10 weeks, more particularly between about ≥8 and about ≤10 weeks, after the last dose of the VEGF antagonist B was administered to the patient,
(b) followed by administering one or more doses of the VEGF antagonist A (e.g., aflibercept or ranibizumab) with an administration interval between about 4 and about 12 weeks, in particular between about 4 and about 8 weeks,
   wherein the VEGF antagonist B is brolucizumab,
   and in particular wherein the VEGF antagonist A is aflibercept or ranibizumab, and
   more particularly wherein the dose of aflibercept (e.g., the initial dose and the following doses) is about 2 mg, in particular 2 mg or wherein the dose of ranibizumab (e.g., the initial dose and the following doses) is about 0.5 mg, in particular 0.5 mg.

In a more specific embodiment, the present disclosure provides methods and uses for treating neovascular age-related macular degeneration (nAMD) in a patient that has previously received one or more doses of a VEGF antagonist B (Compound B) or in a patient pretreated with one or more doses of a VEGF antagonist B (Compound B), the method or the use comprising:
(a) discontinuing treatment with VEGF antagonist B, or replacing VEGF antagonist B with a VEGF antagonist A (Compound A), e.g., aflibercept or ranibizumab,
(b) administering to the patient the initial dose of a VEGF antagonist A between about ≥4 and about ≤14 weeks, e.g., about ≥4 and about ≤12 weeks (e.g., between about one month and three months), about ≥4 and about ≤10 weeks, in particular between about ≥6 and about ≤10 weeks, more particularly between about ≥8 and about ≤10 weeks, after the last dose of the VEGF antagonist B was administered to the patient,
(c) followed by administering one or more doses of the VEGF antagonist A (e.g., aflibercept or ranibizumab) with an administration interval between about 4 and about 12 weeks, in particular between about 4 and about 8 weeks,
   wherein the VEGF antagonist B is brolucizumab,
   and in particular wherein the VEGF antagonist A is aflibercept or ranibizumab, and
   more particularly wherein the dose of aflibercept (e.g., the initial dose and the following doses) is about 2 mg, in particular 2 mg or wherein the dose of ranibizumab (e.g., the initial dose and the following doses) is about 0.5 mg, in particular 0.5 mg.

In one embodiment, the present disclosure provides methods and uses for treating diabetic macular edema (DME) in a patient that has previously received one or more doses of a VEGF antagonist B (Compound B), e.g., aflibercept or ranibizumab, or in a patient pretreated with one or more doses of a VEGF antagonist B (Compound A), e.g., aflibercept or ranibizumab, the method or the use comprising administering to the patient the initial dose of a VEGF antagonist A (Compound A), e.g., brolucizumab, followed by one or more doses of the VEGF antagonist A with an administration interval between about 8 and about 12 weeks, in particular wherein the VEGF antagonist A is brolucizumab, and more particularly wherein the dose of the VEGF antagonist A (e.g., the initial dose and the following doses) is from about 3 mg to about 6 mg, in particular about 3 mg or about 6 mg, more particularly 6 mg.

In a more specific embodiment, the present disclosure provides methods and uses for treating diabetic macular edema (DME) in a patient that has previously received one or more doses of a VEGF antagonist B (Compound B), e.g., aflibercept or ranibizumab, or in a patient pretreated with one or more doses of a VEGF antagonist B (Compound B), e.g., aflibercept or ranibizumab, the method or the use comprising:
(a) administering to the patient the initial dose of a VEGF antagonist A (Compound A), e.g., brolucizumab, between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B (e.g., aflibercept or ranibizumab) was administered to the patient,
(b) followed by administering one or more doses of the VEGF antagonist A with an administration interval between about 8 and about 12 weeks,
   in particular wherein the VEGF antagonist A is brolucizumab, and
   more particularly wherein the dose of the VEGF antagonist A (e.g., the initial dose and the following doses) is from about 3 mg to about 6 mg, in particular about 3 mg or about 6 mg, more particularly 6 mg.

In a more specific embodiment, the present disclosure provides methods and uses for treating diabetic macular edema (DME) in a patient that has previously received one or more doses of a VEGF antagonist B (Compound B), e.g., aflibercept or ranibizumab, or in a patient pretreated with one or more doses of a VEGF antagonist B (Compound B), e.g., aflibercept or ranibizumab, the method or the use comprising:
(a) discontinuing treatment with VEGF antagonist B, or replacing VEGF antagonist B with a VEGF antagonist A (Compound A), e.g., brolucizumab;
(b) administering to the patient the initial dose of the VEGF antagonist A (e.g., brolucizumab) between about ≥4 and about ≤24 weeks (e.g., between about one month and 6 months), e.g., about ≥4 and about ≤21 weeks (e.g., between about one month and 5 months), about ≥4 and about ≤18 weeks, about ≥4 and about ≤16 weeks (e.g., between about one month and 4 months), about ≥4 and about ≤12 weeks (e.g., between about one month and 3 months), in particular between about ≥4 and about ≤10 weeks, more particularly between about ≥6 and about ≤10 weeks, after the last dose of the VEGF antagonist B was administered to the patient;
(c) followed by administering one or more doses of the VEGF antagonist A with an administration interval between about 8 and about 12 weeks,
   in particular wherein the VEGF antagonist A is brolucizumab, and
   more particularly wherein the dose of the VEGF antagonist A (e.g., the initial dose and the following doses) is from about 3 mg to about 6 mg, in particular about 3 mg or about 6 mg, more particularly 6 mg.

In another embodiment, the present disclosure provides methods and uses for treating neovascular age-related macular degeneration (nAMD) in a patient that has previously received one or more doses of a VEGF antagonist B (Compound B), e.g., brolucizumab, or in a patient pretreated with one or more doses of a VEGF antagonist B (Compound B), e.g., brolucizumab, the method or the use comprising administering to the patient the initial dose of a VEGF antagonist A (Compound A), e.g., aflibercept or ranibizumab, followed by one or more doses of the VEGF antagonist A with an administration interval between about 8 and about 12 weeks, preferably 8 weeks. Suitably, the VEGF antagonist A is aflibercept. Suitably, the VEGF antagonist A is aflibercept and the dose of the VEGF antagonist A (e.g., the initial dose and the following doses) is about 2 mg. Suitably, the VEGF antagonist A is ranibizumab. Suitably, the VEGF antagonist A is ranibizumab and the dose of the VEGF antagonist A (e.g., the initial dose and the following doses) is about 0.5 mg.

In certain embodiment, the maintenance phase starts with a dosing regimen wherein the VEGF antagonist A (Compound A), e.g., brolucizumab, is administered once every 12 weeks (q12w), and the dosing interval is adjusted (e.g., plus or minus 2, 3, 4, 5, 6, 7, 8, 10, 11 or 12 weeks) depending on a disease activity assessment. For example, if disease activity is observed prior to administering a q12w dose, the patient will receive the q12w dose as planned, and receive the next dose 8 weeks later, thus being placed on a q8w dosing regimen until disease activity is no longer observed. When disease activity is no longer observed, the dosing regimen will be adjusted back to a q12w schedule. In another embodiment, if no disease activity is observed at any time during the maintenance phase, the treatment interval may be extended by 4 weeks to a q16w. If disease activity is observed in a patient on a q16w or other dosing regimen more than q12w, the treatment interval may be adjusted back to a q12w dosing regimen.

In certain embodiments, a Disease Activity Assessment (DAA) is conducted at all scheduled treatment visits. At assessment weeks, the patients can be currently on, for example, a 8-week or 12-week or 16-week interval regimen. The assessment can determine if a patient stays on the current interval or switches to a different interval. For example, a patient is reassigned to q8w or q12w or q16w dosing regimen based on the presence of the disease activity as determined by a treatment provider.

Suitably, the disease activity may be assessed based on visual function, retinal structure and leakage. An assessment as described herein preferably includes one or more of the following tests to assess activity of a VEGF antagonist (e.g., brolucizumab) on visual function, retinal structure and leakage: (i) best corrected visual acuity (BCVA), e.g., best corrected visual acuity with ETDRS-like chart at 4 meters, (ii) visual acuity (VA), (iii) central subfield thickness (CSFT), (iv) presence of intraretinal cysts/fluid, (v) ETDRS DRSS score based on 7-field stereo Color Fundus Photography (CFP), (vi) anatomical retinal evaluation by Optical Coherence Tomography (OCT), standard or wide-field Fluorescein Angiography (FA), OCT angiography, and/or wide-field CFP/FA, (vii) peripheral visual field assessed by perimetry, (viii) contrast sensitivity, (viii) severity of diabetic retinopathy.

Suitably, the disease activity may be assessed based on one or more of the following: (i) best corrected visual acuity (BCVA), (ii) visual acuity (VA), (iii) central subfield thickness (CSFT), and (iv) presence of intraretinal cysts/fluid. The presence of ocular disease activity, in particular nAMD disease activity, includes one or more of the following: (i) decrease in Best Corrected Visual Acuity (BCVA), (ii) decrease in Visual Acuity (VA), (iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF). Fluid measured in the eye can be intraretinal and/or subretinal fluid.

In specific embodiments, the presence of ocular disease activity, in particular nAMD disease activity, includes one or more of the following:
(i) decrease in BCVA of ≥2 letters, e.g., decrease in BCVA of ≥3 letters, decrease in BCVA of ≥4 letters, in particular decrease in BCVA of ≥5 letters, more particularly wherein:
   the decrease in BCVA is observed at Week 8 or Week 12 after the last administration of the VEGF antagonist A (e.g., brolucizumab) compared to a baseline BCVA, wherein the baseline BCVA was assessed prior to the last administration of the VEGF antagonist A, or
   the decrease in BCVA is observed after 4 months or longer, e.g., after 6 months or longer, of the administration of the VEGF antagonist A compared to a baseline BCVA, wherein the baseline BCVA was assessed 4 months or longer, e.g., 6 months or longer, prior to the last administration of the VEGF antagonist A;
(ii) decrease in VA of ≥1 letters, e.g., decrease in VA of ≥2 letters, in particular decrease in VA of ≥3 letters, more particularly wherein:
   the decrease in VA is observed at Week 8 or Week 12 after the last administration of the VEGF antagonist A (e.g., brolucizumab) compared to a baseline VA, wherein the baseline VA was assessed prior to the last administration of the VEGF antagonist A, or
   the decrease in VA is observed after 4 months or longer, e.g., after 6 months or longer, of the administration of the VEGF antagonist A compared to a baseline VA, wherein the baseline VA was assessed 4 months or longer, e.g., 6 months or longer, prior to the last administration of the VEGF antagonist A;
(iii) CSFT increase ≥25 μm, e.g., CSFT increase ≥50 μm, in particular CSFT increase ≥75 μm, more particularly wherein:
   the CSFT increase is observed at Week 8 or Week 12 after the last administration of the VEGF antagonist A (e.g., brolucizumab) compared to a baseline CSFT, wherein the baseline CSFT was assessed prior to the last administration of the VEGF antagonist A, or
   the CSFT increase is observed after 4 months or longer, e.g., after 6 months or longer, of the administration of the VEGF antagonist A compared to a baseline CSFT, wherein the baseline CSFT was assessed 4 months or longer, e.g., 6 months or longer, prior to the last administration of the VEGF antagonist A;
(iv) new or persistent or recurrent intraretinal cysts (IRC) and/or intraretinal fluid (IRF) and/or subretinal fluid (SRF), in particular wherein:
   new or persistent or recurrent intraretinal cysts (IRC) and/or intraretinal fluid (IRF) and/or subretinal fluid (SRF) are observed at at Week 8 or Week 12 after the last administration of the VEGF antagonist A (e.g., brolucizumab) compared to a baseline RC and/or IRF and/or SRF, wherein the baseline RC and/or IRF and/or SRF was assessed prior to the last administration of the VEGF antagonist A, or
   new or persistent or recurrent intraretinal cysts (IRC) and/or intraretinal fluid (IRF) and/or subretinal fluid (SRF) are observed after 4 months or longer, e.g., after 6 months or longer, of the administration of the VEGF antagonist A compared to a baseline IRC and/or IRF and/or SRF, wherein the baseline IRC and/or IRF and/or SRF was assessed 4 months or longer, e.g., 6 months or longer, prior to the last administration of the VEGF antagonist A.

Where disease activity is present (for example, loss of letters measured by BCVA, increase in CST, increased fluid accumulation, and or increased severity of ocular disease compared with baseline reading for the patient or compared with any previous assessment), a more frequent dosing interval is prescribed going forward. Where improvement of disease activity is observed, a less frequent dosing interval is prescribed.

Suitably, the methods and uses of the present disclosure comprise administering to the patient one or more doses of a VEGF antagonist A (e.g., brolucizumab) according to “treat-to-control” (TcT) dosing regimen concept, e.g., wherein a dosing interval is adjusted based on disease activity to meet the patient's needs, including shorting, maintaining or extending the treatment interval based on disease activity. The Treat-to-Control (TtC) regimen entails sustained disease control to determine the optimal treatment interval for each patient. For example, the Treat-to-Control (TtC) dosing regimen entails increasing or decreasing a dosing interval in 2 weeks or 4 weeks steps.

Suitably, the methods and uses of the present disclosure comprise administering to the patient one or more doses of a VEGF antagonist A (e.g., brolucizumab) according to Treat-and-Extend (T&E) dosing regimen concepts (Wykoff et al., 2018). For example, the Treat-and-Extend (T&E) dosing regimen entails increasing or decreasing a dosing interval in 2 weeks steps.

In certain embodiments, the dosing frequency is adjusted based on the outcome of disease activity assessments, for example using pre-defined visual and anatomic criteria. In one embodiment, dosing frequency of the VEGF antagonist A (e.g., brolucizumab) can be adjusted by decreasing the dosing interval from once every 24 weeks (q24w) to once every 18 weeks (q18w). In one embodiment, dosing frequency of the VEGF antagonist A (e.g., brolucizumab) can be adjusted by decreasing the dosing interval from once every 18 weeks (q18w) to once every 12 weeks (q12w). In one embodiment, dosing frequency of the VEGF antagonist A (e.g., brolucizumab) can be adjusted by decreasing the dosing interval from once every 12 weeks (q12w) to once every 8 weeks (q8w) or to once every 6 weeks (q6w) based on the disease activity assessment at any scheduled treatment visit. In another embodiment, dosing frequency of the VEGF antagonist A (e.g., brolucizumab) can be adjusted by increasing the dosing interval from to once every 6 weeks (q6w) or once every 8 weeks (q8w) to once every 12 weeks (q12w) based on the disease activity assessment at any scheduled treatment visit. In another embodiment, dosing frequency of the VEGF antagonist A (e.g., brolucizumab) can be adjusted by increasing the dosing interval from once every 12 weeks (q12w) or to once every 18 weeks (q18w) or to once every 24 weeks (q24w) based on the disease activity assessment at any scheduled treatment visit. When disease activity is identified as described herein, the treatment regimen can be changed, e.g., from every 12 weeks to every 8 weeks (i.e., q8w). The disclosure provides specific criteria established by the inventors based on disease activity assessments to determine when a shorter administration interval, e.g., an injection interval, should be used and when a longer administration interval, e.g., an injection interval, should be used, e.g., an 8-week interval should be used and when a 12-week interval should be continued. In some cases, a patient might be on a 12-week interval regimen for some time, and then switch to a 8-week interval, and then switch back to the 12-week interval. Thus, patients may not stay on one interval regimen, and may go back and forth depending on assessments according to the criteria set forth herein.

In one embodiment, assessments of disease activity to establish the patient's disease status occurs at baseline (e.g., Week 0; first treatment with a VEGF antagonist B; first treatment with a VEGF antagonist A; prior to the last administration of the VEGF antagonist B; prior to the last administration of the VEGF antagonist A). The assessment of the disease activity (DAA) during treatment regimens is at the discretion of the person making the assessment (e.g., the treatment provider), and is based on changes in vision and anatomical and morphological and clinical parameters with reference to the patients' baseline disease status (e.g., at Week 0; first treatment with a VEGF antagonist B; first treatment with a VEGF antagonist A; prior to the last administration of the VEGF antagonist B; prior to the last administration of the VEGF antagonist A).

In certain other embodiments, during the maintenance phase, the VEGF antagonist is administered on an as needed basis, i.e., pro re nata (PRN), at the discretion of a treatment provider (e.g., a physician or other qualified medical professional) based on visual and/or anatomical outcomes to determine disease activity.

In one embodiment, the Compound A or the VEGF antagonist A or the VEGF antagonist “A” of the disclosure is any licensed anti-VEGF drug such as brolucizumab, ranibizumab or aflibercept. In one embodiment, the Compound A or the VEGF antagonist A or the VEGF antagonist “A” of the disclosure is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept). In a preferred embodiment, the Compound A or the VEGF antagonist A of the disclosure is brolucizumab or ranibizumab or aflibercept. In a more preferred embodiment, the Compound A or the VEGF antagonist A of the disclosure is brolucizumab.

In one embodiment, the Compound B or the VEGF antagonist B or the VEGF antagonist “B” of the disclosure is any licensed anti-VEGF drug such as brolucizumab, ranibizumab or aflibercept. In one embodiment, the Compound B or the VEGF antagonist B or the VEGF antagonist “B” of the disclosure is an anti-VEGF antibody (such as brolucizumab or ranibizumab or bevacizumab or a bi-specific antibody such as faricimab) or an anti-VEGF DARPin (such as abicipar) or a soluble VEGF receptor (e.g., a fusion protein composed of the VEGF receptor domains, such as a fusion protein composed of the combination between VEGF receptor domains with the Fc fragment of human immunoglobulin with the Fc fragment of human immunoglobulin, e.g., conbercept, aflibercept) or AAV containing a sequence encoding for an anti-VEGF antibody (such as RGX-314 from Regenxbio), or AAV containing a sequence encoding the VEGF receptor domains, e.g., conbercept (such as ADVM-022 from Adverum) or any licensed anti-VEGF drug (such as brolucizumab, ranibizumab or aflibercept). In a preferred embodiment, the the Compound B or VEGF antagonist B of the disclosure is brolucizumab or ranibizumab or aflibercept. In a more preferred embodiment, the Compound B or the VEGF antagonist B of the disclosure is ranibizumab or aflibercept.

In certain embodiments, the VEGF antagonist of the disclosure (the VEGF antagonist A or the VEGF antagonist B, in particular the VEGF antagonist A) is an anti-VEGF antibody, e.g., a single chain antibody (scFv) or Fab fragment.

In certain embodiments, the VEGF antagonist of the disclosure (the VEGF antagonist A (the Compound A) or the VEGF antagonist B (the Compound B), in particular the VEGF antagonist A) is an anti-VEGF antibody, particularly anti-VEGF antibodies described in WO 2009/155724, the entire contents of which are hereby incorporated by reference.

In one embodiment, the VEGF antagonist of the disclosure (the VEGF antagonist A (the Compound A) or the VEGF antagonist B (Compound B), in particular the VEGF antagonist A) is an anti-VEGF antibody comprising a variable heavy chain having the sequence as set forth in SEQ ID NO: 1 and a variable light chain having the sequence as set forth in SEQ ID NO: 2.

In another embodiment, the VEGF antagonist of the disclosure (the VEGF antagonist A (Compound A) or the VEGF antagonist B (Compound B), in particular the VEGF antagonist A) is an anti-VEGF antibody comprising the sequence as set forth in SEQ ID NO: 3.

In a preferred embodiment, the VEGF antagonist of the disclosure (the VEGF antagonist A (Compound A) or the VEGF antagonist B (Compound B), in particular a VEGF antagonist A) is brolucizumab (which comprises the sequence of SEQ ID NO: 3). The sequence of brolucizumab is set forth in SEQ ID NO: 4. A methionine derived from the start codon in an expression vector is present in the final protein in cases where it has not been cleaved posttranslationally as follows.

In another embodiment, the VEGF antagonist of the disclosure (the VEGF antagonist A (Compound A) or the VEGF antagonist B (Compound B), in particular the VEGF antagonist A) is an anti-VEGF antibody comprising three light chain CDRs (CDRL1, CDRL2, and CDRL3) and three heavy chain CDRs (CDRH1, a CDRH2, a CDRH3) as follows:

CDRL1
SEQ ID NO: 5
QASEIIHSWLA
CDRL2
SEQ ID NO: 6
LASTLAS
CDRL3
SEQ ID NO: 7
QNVYLASTNGAN
CDRH1
SEQ ID NO: 8
GFSLTDYYYMT
CDRH2
SEQ ID NO: 9
FIDPDDDPYYATWAKG
CDRH3
SEQ ID NO: 10
GDHNSGWGLDI

In a preferred embodiment, the Compound A or the VEGF antagonist A of the disclosure is brolucizumab, and the Compound B or the VEGF antagonist B of the disclosure is ranibizumab or aflibercept.

In certain embodiments, the VEGF antagonist of the disclosure, e.g., the VEGF antagonist A or VEGF antagonist B, is administered by an injection, e.g., an intravitreal injection.

In some embodiments, the VEGF antagonist of the disclosure, in particular the VEGF antagonist A, is brolucizumab and is administered at a dose of about 1, about 2, about 3, about 4, about 5, or about 6 mg (e.g., about 6 mg/0.05 mL) as an intravitreal injection. In certain embodiments, the VEGF antagonist of the disclosure, in particular the VEGF antagonist A, is brolucizumab and is administered at a dose of 1, 2, 3, 4, 5, or 6 mg (e.g., 6 mg/0.05 mL) as an intravitreal injection.

In some embodiments, the VEGF antagonist of the disclosure, in particular the VEGF antagonist B, is aflibercept and is administered at a dose of about 0.5, about 1 or about 2 mg (e.g., about 2 mg/0.05 mL) as an intravitreal injection. In certain embodiments, the VEGF antagonist of the disclosure, in particular the VEGF antagonist B, is aflibercept and is administered at a dose of 0.5, 1 or 2 mg (e.g., 2 mg/0.05 mL) as an intravitreal injection.

In some embodiments, the VEGF antagonist of the disclosure, in particular the VEGF antagonist B, is aflibercept and is administered at a dose of about 0.2, about 0.3, about 0.4 or about 0.5 mg (e.g., about 0.5 mg/0.05 mL) as an intravitreal injection. In certain embodiments, the VEGF antagonist of the disclosure, in particular the VEGF antagonist B, is aflibercept and is administered at a dose of 0.2, 0.3, 0.4 or 0.5 mg (e.g., 0.5 mg/0.05 mL) as an intravitreal injection.

### Pharmaceutical Preparations

In one aspect, the methods or uses of the disclosure comprise the use of pharmaceutical formulations or pharmaceutical compositions comprising a VEGF antagonist, e.g., an anti-VEGF antibody. The term “pharmaceutical formulation” or “pharmaceutical composition” refers to preparations which are in such form as to permit the biological activity of the antagonist, e.g., antibody or antibody derivative, to be unequivocally effective, and which contain no additional components which are toxic to the subjects to which the formulation or composition would be administered. “Pharmaceutically acceptable” excipients (vehicles, additives) are those which can reasonably be administered to a subject mammal to provide an effective dose of the active ingredient employed.

A “stable” formulation is one in which a therapeutic agent, e.g. a VEGF antagonist, e.g., an anti-VEGF antibody or antibody derivative thereof, essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable at room temperature (about 30° C.) or at 40° C. for at least 1 week and/or stable at about 2-8° C. for at least 3 months to 2 years. Furthermore, the formulation is preferably stable following freezing (to, e.g., −70° C.) and thawing of the formulation.

An antagonist, e.g., an antibody or antibody derivative, “retains its physical stability” in a pharmaceutical formulation if it meets the defined release specifications for aggregation, degradation, precipitation and/or denaturation upon visual examination of color and/or clarity, or as measured by UV light scattering or by size exclusion chromatography, or other suitable art recognized methods.

An antagonist, e.g., an antibody or antibody derivative, “retains its chemical stability” in a pharmaceutical formulation, if the chemical stability at a given time is such that the compound, e.g., protein, is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antagonist, e.g., an antibody or antibody derivative, “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example. Other “biological activity” assays for antibodies are elaborated herein below.

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

A “polyol” is a substance with multiple hydroxyl groups, and includes sugars (reducing and non-reducing sugars), sugar alcohols and sugar acids. Preferred polyols herein have a molecular weight which is less than about 600 kD (e.g. in the range from about 120 to about 400 kD). A “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Non-reducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. Where it is desired that the formulation is freeze-thaw stable, the polyol is preferably one which does not crystallize at freezing temperatures (e.g. −20° C.) such that it destabilizes the antibody in the formulation. Non-reducing sugars such as sucrose and trehalose are the preferred polyols herein, with trehalose being preferred over sucrose, because of the superior solution stability of trehalose.

As used herein, “buffer” refers to a buffered solution that resists changes in pH by the action of its acid-base conjugate components. The buffer of this disclosure has a pH in the range from about 4.5 to about 8.0; preferably from about 5.5 to about 7. Examples of buffers that will control the pH in this range include acetate (e.g. sodium acetate), succinate (such as sodium succinate), gluconate, histidine, citrate and other organic acid buffers. Where a freeze-thaw stable formulation is desired, the buffer is preferably not phosphate.

In a pharmacological sense, in the context of the present disclosure, a “therapeutically effective amount” of a therapeutic agent, e.g., a VEGF antagonist, e.g., an anti-VEGF antibody or antibody derivative, refers to an amount effective in the prevention or treatment of a disorder for the treatment of which the antagonist, e.g., antibody or antibody derivative, is effective. This includes chronic and acute disorders or diseases including those pathological conditions which predispose the mammal to the disorder in question.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The pharmaceutical compositions used in present disclosure comprise a VEGF antagonist A, preferably an anti-VEGF antibody (e.g., an anti-VEGF antibody comprising the variable light chain sequence of SEQ ID NO: 1 and the variable heavy chain sequence of SEQ ID NO: 2, such as brolucizumab), together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. As noted above, other active ingredients may (but need not) be included in the pharmaceutical compositions provided herein.

A carrier is a substance that may be associated with an antibody or antibody derivative prior to administration to a patient, often for the purpose of controlling stability or bioavailability of the compound. Carriers for use within such formulations are generally biocompatible, and may also be biodegradable. Carriers include, for example, monovalent or multivalent molecules such as serum albumin (e.g., human or bovine), egg albumin, peptides, polylysine and polysaccharides such as aminodextran and polyamidoamines. Carriers also include solid support materials such as beads and microparticles comprising, for example, polylactate polyglycolate, poly(lactide-co-glycolide), polyacrylate, latex, starch, cellulose or dextran. A carrier may bear the compounds in a variety of ways, including covalent bonding (either directly or via a linker group), noncovalent interaction or admixture.

Pharmaceutical compositions may be formulated for any appropriate manner of administration, including, for example, topical, intraocular, oral, nasal, rectal or parenteral administration. In certain embodiments, compositions in a form suitable for intraocular injection, such as intravitreal injection, are preferred. Other forms include, for example, pills, tablets, troches, lozenges, aqueous or oily suspensions, dispersible powders or granules, emulsion, hard or soft capsules, or syrups or elixirs. Within yet other embodiments, compositions provided herein may be formulated as a lyophilizate. The term parenteral as used herein includes subcutaneous, intradermal, intravascular (e.g., intravenous), intramuscular, spinal, intracranial, intrathecal and intraperitoneal injection, as well as any similar injection or infusion technique.

The pharmaceutical composition may be prepared as a sterile injectable aqueous or oleaginous suspension in which the active agent (i.e. VEGF antagonist), depending on the vehicle and concentration used, is either suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid may be used in the preparation of injectable compositions, and adjuvants such as local anesthetics, preservatives and/or buffering agents can be dissolved in the vehicle.

An aqueous formulation of a VEGF antagonist, e.g., a VEGF antagonist A, e.g., an anti-VEGF antibody (e.g., brolucizumab), used in the methods or uses of the disclosure is prepared in a pH-buffered solution. Preferably, the buffer of such aqueous formulation has a pH in the range from about 4.5 to about 8.0, preferably from about 5.5 to about 7.0, most preferably about 6.75. In one embodiment, the pH of an aqueous pharmaceutical composition of the disclosure is about 7.0-7.5, or about 7.0-7.4, about 7.0-7.3, about 7.0-7.2, about 7.1-7.6, about 7.2-7.6, about 7.3-7.6 or about 7.4-7.6. In one embodiment, an aqueous pharmaceutical composition of the disclosure has a pH of about 7.0, about 7.1, about 7.2, about 7.3, about 7.4, about 7.5 or about 7.6. In a preferred embodiment, the aqueous pharmaceutical composition has a pH of ≥7.0 In a preferred embodiment, the aqueous pharmaceutical composition has a pH of about 7.2. In another preferred embodiment, the aqueous pharmaceutical composition has a pH of about 7.4. In another preferred embodiment, the aqueous pharmaceutical composition has a pH of about 7.6. Examples of buffers that will control the pH within this range include acetate (e.g. sodium acetate), succinate (such as sodium succinate), gluconate, histidine, citrate and other organic acid buffers. The buffer concentration can be from about 1 mM to about 50 mM, preferably from about 5 mM to about 30 mM, depending, for example, on the buffer and the desired isotonicity of the formulation.

A polyol, which acts as a tonicifier, may be used to stabilize an antibody in an aqueous formulation. In preferred embodiments, the polyol is a non-reducing sugar, such as sucrose or trehalose. If desired, the polyol is added to the formulation in an amount that may vary with respect to the desired isotonicity of the formulation. Preferably the aqueous formulation is isotonic, in which case suitable concentrations of the polyol in the formulation are in the range from about 1% to about 15% w/v, preferably in the range from about 2% to about 10% w/v, for example. However, hypertonic or hypotonic formulations may also be suitable. The amount of polyol added may also alter with respect to the molecular weight of the polyol. For example, a lower amount of a monosaccharide (e.g. mannitol) may be added, compared to a disaccharide (such as trehalose).

A surfactant is also added to an aqueous antibody formulation. Exemplary surfactants include nonionic surfactants such as polysorbates (e.g. polysorbates 20, 80 etc) or poloxamers (e.g. poloxamer 188). The amount of surfactant added is such that it reduces aggregation of the formulated antibody/antibody derivative and/or minimizes the formation of particulates in the formulation and/or reduces adsorption. For example, the surfactant may be present in the formulation in an amount from about 0.001% to about 0.5%, preferably from about 0.005% to about 0.2% and most preferably from about 0.01% to about 0.1%.

In one embodiment, an aqueous antibody formulation used in the methods or uses of the disclosure is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, most preferably from about 0.5% to about 1%. One or more other pharmaceutically acceptable carriers, excipients or stabilizers such as those described in Remington's Pharmaceutical Sciences 21st edition, Osol, A. Ed. (2006) may be included in the formulation provided that they do not adversely affect the desired characteristics of the formulation. Acceptable carriers, excipients or stabilizers are non-toxic to recipients at the dosages and concentrations employed and include: additional buffering agents, co-solvents, antioxidants including ascorbic acid and methionine, chelating agents such as EDTA, metal complexes (e.g. Zn-protein complexes), biodegradable polymers such as polyesters, and/or salt-forming counterions such as sodium.

Formulations to be used for in vivo administration must be sterile. This is readily accomplished by filtration through sterile filtration membranes, prior to, or following, preparation of the formulation.

In one embodiment, the VEGF antagonist of the disclosure is administered to an eye of a subject in need of treatment in accordance with known methods for ocular delivery. Preferably, the subject is a human, the VEGF antagonist A is an anti-VEGF antibody (preferably brolucizumab), and the antibody is administered directly to an eye. Administration to a patient can be accomplished, for example, by intravitreal injection.

The VEGF antagonist in the methods and uses of the disclosure can be administered as the sole treatment or in conjunction with other drugs or therapies useful in treating the condition in question.

A preferred formulation for brolucizumab for intravitreal injection comprises about 4.5% to 11% (w/v) sucrose, 5-20 mM sodium citrate, and 0.001% to 0.05% (w/v) polysorbate 80, wherein the pH of the formulation is about 7.0 to about 7.4. One such formulation comprises 5.9% (w/v) sucrose, 10 mM sodium citrate, 0.02% (w/v) polysorbate 80, pH of 7.2, and 6 mg of brolucizumab. Another such formulation comprises 6.4% (w/v) or 5.8% sucrose, 12 mM or 10 mM sodium citrate, 0.02% (w/v) polysorbate 80, pH of 7.2, and 3 mg of brolucizumab. Preferred concentrations of brolucizumab are about 120 mg/ml and about 60 mg/ml. Doses can be delivered, for example as 6 mg/50 μL and 3 mg/50 μL concentrations.

### Dosage

A dose used in the methods or uses of the disclosure is based on the specific disease or condition being treated, and is a therapeutically effective dose. Amounts effective for this use will depend upon the severity of the disorder being treated and the general state of the patient's own immune system. The dose amount can be readily determined using known dosage adjustment techniques by a physician having ordinary skill in treatment of the disease or condition. The therapeutically effective amount of a VEGF antagonist used in the methods or uses of the disclosure is determined by taking into account the desired dose volumes and mode(s) of administration, for example. Typically, therapeutically effective compositions are administered in a dosage ranging from 0.001 mg/ml to about 200 mg/ml per dose.

In one embodiment of the present disclosure, the VEGF antagonist, in particular the VEGF antagonist A, used in the methods or uses of the disclosure is brolucizumab, and a dosage thereof used in the methods or uses of the disclosure is about 60 mg/ml to about 120 mg/ml (for example, a dosage is 60, 70, 80, 90, 100, 110, or 120 mg/ml). In a preferred embodiment, the dosage of the VEGF antagonist A used in the methods or uses of the disclosure is 60 mg/ml or 120 mg/ml.

In certain embodiments, a dose of the VEGF antagonist, e.g., VEGF antagonist A or VEGF antagonist B, is administered directly to an eye of a patient. In one embodiment, a dose of the VEGF antagonist, e.g., VEGF antagonist A or VEGF antagonist B, per eye is at least about 0.5 mg up to about 6 mg. Preferred doses per eye include about 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, and 6.0 mg. In one embodiment, a dose per eye is at least about 3 mg up to about 6 mg, in particular about 3 mg or about 6 mg. Doses can be administered in various volumes suitable for ophthalmic administration, such as 50 μl or 100 μl, for example, including 3 mg/50 μl or 6 mg/50 μl. Smaller volumes can also be used, including 20 μl or less, for example about 20 μl, about 10 μl, or about 8.0 μl. In certain embodiments, a dose of 2.4 mg/20 μl, 1.2 mg/10 μl or 1 mg/8.0 μl (e.g., 1 mg/8.3 μl) is delivered to an eye of a patient for treating or ameliorating one or more of the diseases and disorders described above. Delivery can be, for example, by an injections, e.g., an intravitreal injection.

In specific embodiments, the VEGF antagonist of the disclosure, in particular the VEGF antagonist A, is brolucizumab and is administered at a dose of about 1, about 2, about 3, about 4, about 5, or about 6 mg (e.g., about 6 mg/0.05 mL), e.g., 1, 2, 3, 4, 5, or 6 mg (e.g., 6 mg/0.05 mL), as an injections, e.g., an intravitreal injection.

In specific embodiments, the VEGF antagonist of the disclosure, in particular the VEGF antagonist B, is aflibercept, and is administered at a dose of about 0.5, about 1 or about 2 mg (e.g., about 2 mg/0.05 mL), e.g., 0.5, 1 or 2 mg (e.g., 2 mg/0.05 mL), as an injections, e.g., an intravitreal injection.

In specific embodiments, the VEGF antagonist, in particular the VEGF antagonist B, used in the methods or uses of the disclosure is aflibercept, and is administered at a dose of about 0.2, about 0.3, about 0.4 or about 0.5 mg (e.g., about 0.5 mg/0.05 mL), e.g., 0.2, 0.3, 0.4 or 0.5 mg (e.g., 0.5 mg/0.05 mL), as an injections, e.g., an intravitreal injection.

### Kits

The disclosure also provides a kit, comprising: a drug container (e.g., a vial or a prefilled syringe) comprising a VEGF antagonist (e.g., brolucizumab), and instructions for using the VEGF antagonist for treating a patient diagnosed with ocular disease, e.g., nAMD. In one embodiment, the instructions indicate the VEGF antagonist (e.g., brolucizumab) is to be administered to a patient as an initial dose followed by one or more doses of the VEGF antagonist every 8 to 24 weeks, e.g., every 8 to 12 weeks. In a more specific embodiment, the instructions indicate the VEGF antagonist (e.g., brolucizumab) is to be administered to a patient as an initial dose followed by one or more doses of the VEGF antagonist every 8 to 24 weeks, e.g., every 8 to 12 weeks, at a dose of 3 mg or 6 mg, e.g., 6 mg. In one embodiment, the instructions indicate that a VEGF antagonist treatment can be initiated with one initial injection of a VEGF antagonist followed by treatment intervals every 8 to 24 weeks, e.g., every 8 weeks to every 12 weeks, e.g., every 8 weeks or every 12 weeks. In a more specific embodiment, the instructions indicate that a VEGF antagonist treatment can be initiated with one initial injection of a VEGF antagonist followed by treatment intervals every 8 to 24 weeks, e.g., every 8 weeks to every 12 weeks, e.g., every 8 weeks or every 12 weeks, at a dose of 3 mg or 6 mg, e.g., 6 mg.

The disclosure also provides a kit, comprising: a drug container (e.g., a vial or a prefilled syringe) comprising the VEGF antagonist A (e.g., brolucizumab), and instructions for using the VEGF antagonist A for treating a patient diagnosed with ocular disease, in particular neovascular age-related macular degeneration (nAMD). In one embodiment, the instructions indicate the VEGF antagonist A (e.g., brolucizumab) is to be administered to a patient pretreated with one or more doses of a VEGF antagonist B as follows: discontinue treatment with VEGF antagonist B and instead administer to the patient one or more doses of the VEGF antagonist A (e.g., brolucizumab) every 8 to 12 weeks, in particular at a dose of 3 mg or 6 mg, preferably 6 mg. In one embodiment, the instructions indicate that the VEGF antagonist A (e.g., brolucizumab) treatment for patients pretreated with other VEGF treatments can be initiated with one initial injection of the VEGF antagonist A followed by treatment intervals every 8 weeks to every 12 weeks, in particular every 12 weeks, and/or as individualized by a physician based on a disease activity assessment, in particular at a dose of 3 mg or 6 mg, preferably 6 mg.

In one embodiment, the kit comprises one or more 3 mg or 6 mg doses of brolucizumab, each dose provided in a single use container, e.g., vial, containing sufficient brolucizumab to deliver a 3 mg or 6 mg, preferably 6 mg, dose when administering a volume of 0.05 mL or in a prefilled syringe containing 3 mg or 6 mg, preferably 6 mg of brolucizumab.

In one embodiment, the instructions further indicate a treatment provider (e.g., a physician or other qualified medical professional) can adjust the dosing interval from once every 12 weeks to once every 8 weeks if disease activity is observed in the treated eye.

In another embodiment, the instructions further indicate a treatment provider (e.g., a physician or other qualified medical professional) can extend the dosing interval from once every 8 weeks to once every 12 weeks, if no disease activity is observed in the treated eye.

In yet another embodiment, the instructions further indicate the VEGF antagonist is administered on an as needed basis, i.e., pro re nata (PRN), at the discretion of a treatment provider (e.g., a physician or other qualified medical professional) based on visual and/or anatomical outcomes to determine disease activity during the maintenance phase.

The various features, aspects and embodiments of the present invention, referred to in individual sections above apply, as appropriate, to other sections, mutatis mutandis. Consequently features specified in one section may be combined with features specified in other sections, as appropriate.

Those skilled in the art will recognize, or be able to ascertain using no more than routine experimentation, many equivalents to the specific aspects and embodiments of the invention described herein. Such equivalents are intended to be encompassed by the following claims.

All references cited herein, including patents, patent applications, papers, publications, text books, and the like, and the references cited therein, to the extent that they are not already, are hereby incorporated herein by reference in their entirety

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1: Model Simulations of Drug Concentration Over Time and the Level of Free VEGF

### Methods

A three compartment PKPD model that describes distribution of drug within the eye following IVT administration (pharmacokinetics; PK) and the dynamics of VEGF production and inhibition by drug (pharmacodynamics; PD) was constructed using the MATLAB SimBiology software (MathWorks®, Natick, MA USA). Simulations were performed according to manufacturer's instructions and using computational modeling techniques which are common in the art. The PKPD model was adapted from an example published in the literature (Hutton-Smith et. al., Mol. Pharmaceutics 2018, 15, 2770-2784) that describes PKPD within the human eye for ranibizumab. The adapted model further allows for simulations in which one or two unique VEGF antagonists are administered at specified doses and times. VEGF is present in the eye in a dimeric form consisting of two VEGF monomers and can thus form complexes with one VEGF antagonist or two VEGF antagonists. It should be appreciated that the presence of two different VEGF antagonists in the eye may allow for the formation of heterogenous complexes consisting of one VEGF dimer bound to one molecule of VEGF antagonist A and to one molecule of VEGF antagonist B. The model includes additional reactions to enable association and dissociation of these heterogeneous complexes and their distribution within and out of the eye, following the general formalism of the reactions for homogenous complexes containing one VEGF dimer bound to two molecules of the same VEGF antagonist.

The model contains 3 compartments (Table 1). Following IVT administration of drug into the vitreous compartment, drug distributes between the retina, vitreous, and aqueous compartments and irreversibly distributes out of the eye via the retina and aqueous compartments. The term “ocular half-life” can generally refer to the summed rates at which drug distributes out of the aqueous and retina compartments of the eye (i.e. clears from the eye). VEGF is generally understood to distribute into the eye via the retina and is described within the model as a zero-order reaction in which new VEGF appears in the retina (i.e. “VEGF synthesis” or “VEGF production”). Distribution of VEGF within and out of the eye follows the same formalism as for the VEGF antagonists.

Drug-specific parameters used are defined based upon which drug(s) is included in the specific simulation. These values are shown in Table 2 for brolucizumab, aflibercept, and ranibizumab. The drug:VEGF binding parameter values (the dissociation rate constant “koff” and the equilibrium binding constant “Kd”) were defined on the basis of a surface plasmon resonance (SPR) in vitro binding experiment with drug and recombinant VEGF165 protein at 37° C. according to manufacturer's instructions. The hydrodynamic radius values for drug (“rh_R”) and associated drug:VEGF complexes (“rh_VR” and “rh_RVR”) were sourced from literature reports (Caruso et. al., Mol. Pharmaceutics 2020, 17, 695-709) or from computational structure models of drug and VEGF. This includes estimated hydrodynamic radius values for the heterogenous complexes consisting of one VEGF dimer and two different VEGF antagonists (“rh_ROVR”). Drug-independent parameters used are used to describe VEGF production and distribution within and out of the eye, as well as general biophysical properties of the eye such as permeability coefficients for the Inner Limiting Membrane (ILM) and Retinal Pigment Epithelium (RPE) and the clearance rate from the aqueous chamber. These values are shown in Table 3.

Simulations were performed with drug administration regimens as indicated in each of the figures. The PKPD profiles of the concentration of drug in the vitreous over time (PK) and of free VEGF in the retina (PD) were simulated and results plotted as graphs.

TABLE 1
Model Compartments.

| Name | Value | Units | Description |
|---|---|---|---|
| Ret | 0.22 | mL | Retina |
| Vit | 4.5 | mL | Vitreous |
| Aq | 0.16 | mL | Aqueous |

TABLE 2
Drug-Specific Model Parameters.

| Parameter | Brolucizumab | Aflibercept | Ranibizumab |
|---|---|---|---|
| koff (1/day) | 21.6 | 129.6 | 0.864 |
| Kd (pM) | 101 | 457 | 171 |
| rh_R | 2.27 | 4.50 | 3.00 |
| (nanometer) | | | |
| rh_VR | 3.16 | 5.00 | 3.29 |
| (nanometer) | | | |
| rh_RVR | 3.66 | 6.00 | 4.07 |
| (nanometer) | | | |

| | |
|---|---|
| rh_ROVR | Complex of (brolucizumab:VEGF:ranibizumab) = 3.68 |
| (nanometer) | Complex of (brolucizumab:VEGF:aflibercept) = 5.50 |

TABLE 3
Drug-Independent Model Parameters.

| # | Name | Value | Units | Notes |
|---|---|---|---|---|
| 1 | vin | 17.5 | femtomole/ | VEGF synthesis rate |
| | | | day | (variable value) |
| 2 | cl | 3.600 | milliliter/ | Clearance from the |
| | | | day | aqueous chamber |
| 3 | sret | 9.710 | centimeter\hat{ }2 | Retinal surface area |
| 4 | thalf_V | 4.6366 | day | Ocular half-life of |
| | | | | VEGF |
| 5 | rh_V | 2.390 | nanometer | Hydrodynamic radius |
| | | | | of VEGF |
| 6 | prpe_exp | −0.48 | dimensionless | Used in rule to calculate |
| | | | | RPE permeabilities |
| | | | | (exponent) |
| 7 | pilm_exp | −0.17 | dimensionless | Used in rule to calculate |
| | | | | ILM permeabilities |
| | | | | (exponent) |
| 8 | prpe_factor | 4.04E−07 | centimeter/ | Used in rule to calculate |
| | | | second | RPE permeabilities |
| | | | | (multiplier) |
| 9 | pilm_factor | 2.20E−07 | centimeter/ | Used in rule to calculate |
| | | | second | ILM permeabilities |
| | | | | (multiplier) |

### Results

1. Model Simulations of Drug Concentration Over Time and the Level of Free VEGF in a Patient Treated with a VEGF Antagonist

The level of VEGF production in the eye of a patient was predicted to be a mean value of 17.5 fmol/day with a standard deviation of 6.9 fmol/day (Hutton-Smith et al. Mol. Pharmaceutics, 2018, 15, 2770-2784). To capture differences in levels of VEGF production across patients, the distribution of predicted levels of VEGF production was assumed as Low, Mean, or High as depicted in Table 4.

TABLE 4
VEGF production levels.

| VEGF | | VEGF Synthesis |
|---|---|---|
| Production | Calculation | Rate (fmol/day) |
| Low | mean − 2 * standard deviation | 3.7 |
| Mean | mean | 17.5 |
| High | mean + 2 * standard deviation | 31.3 |

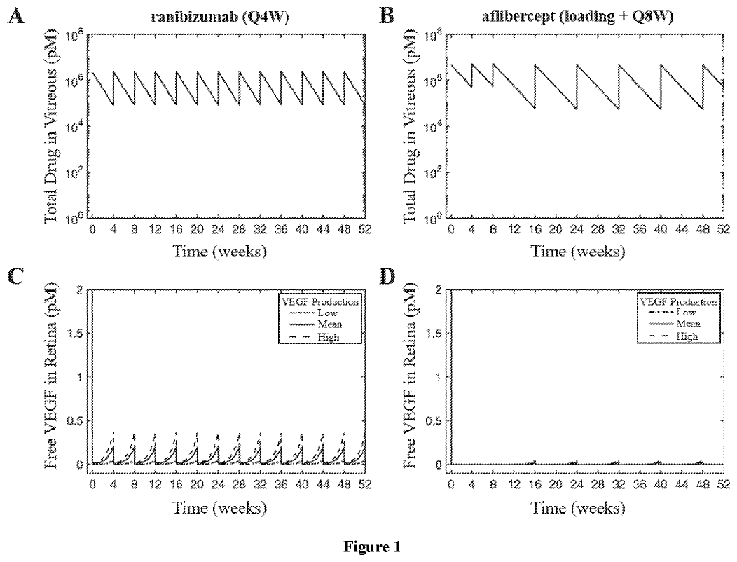

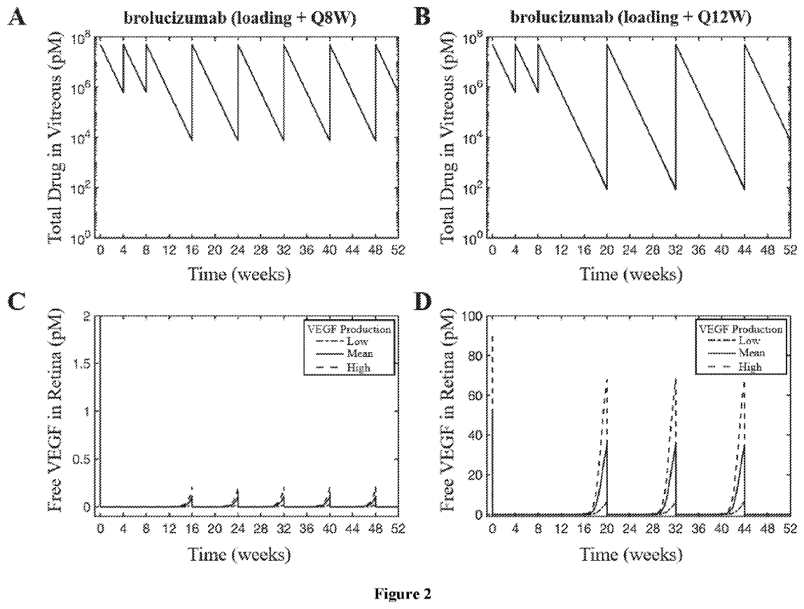

Model simulations of the drug concentration over time (PK) within the vitreous compartment of the eye and the level of free VEGF in the retina compartment of the eye of a patient with Low, Mean, or High predicted levels of VEGF production were performed. Free VEGF in the retina is shown to rapidly decrease following the first drug administration and to incompletely recover before each of the subsequent doses (FIGS. 1 and 2). FIGS. 1A and 1C demonstrate ranibizumab PK in the vitreous and reduction in free VEGF in the retina following IVT administration of 0.5 mg drug every 4 weeks (Q4W). FIGS. 1B and 1D demonstrate aflibercept PK in the vitreous and reduction in free VEGF in the retina following IVT administration of 2.0 mg drug for a loading period consisting of one dose every 4 weeks (Q4W) for the first three months and a maintenance period consisting of one dose every 8 weeks (Q8W). FIGS. 2A and 2C demonstrate brolucizumab PK in the vitreous and reduction in free VEGF in the retina following IVT administration of 6.0 mg drug for a loading period consisting of one dose every 4 weeks (Q4W) for the first three months and a maintenance period consisting of one dose every 8 weeks (Q8W). FIGS. 2B and 2D demonstrate brolucizumab PK in the vitreous and reduction in free VEGF in the retina following IVT administration of 6.0 mg drug for a loading period consisting of one dose every 4 weeks (Q4W) for the first three months and a maintenance period consisting of one dose every twelve weeks (Q12W). The timing and degree of the recovery for free VEGF in the retina is shown to vary based upon the level of VEGF production in the eye of a patient (FIGS. 1 and 2). In addition, it has been demonstrated for brolucizumab that the timing and degree of the recovery for free VEGF in the retina varies based upon both the level of VEGF production in the eye (exemplified as Low, Mean, or High levels of VEGF production) and the dosing interval during the maintenance period (Q8W or Q12W; FIG. 2).

The level of free VEGF in the retina at steady state in the absence of a VEGF antagonist is approximately 50.25 pM (assuming the Mean VEGF Production Rate shown in Table 4). Inhibition thresholds of 5 pM and 10 pM for free VEGF in the retina were defined to represent potential effective levels of drug activity. These values reflect approximate 90% and 80% inhibition of free VEGF in the retina (i.e. approximate IC90 and IC80 values) and these levels are generally understood in the field to reflect significant inhibition of a drug target.

2. Model Simulations of Drug Concentration Over Time and the Level of Free VEGF in a Patient Switched from One VEGF Antagonist to Another

Model simulations were performed for the drug concentration over time (PK) within the vitreous compartment of the eye and the level of free VEGF in the retina compartment of the eye of a patient with Low, Mean, or High predicted levels of VEGF production, in which a patient was administered one VEGF antagonist for a period of approximately one year and after switched to a second VEGF antagonist, which was administered for a subsequent period of approximately one year.

### 2.1 Ranibizumab→Brolucizumab

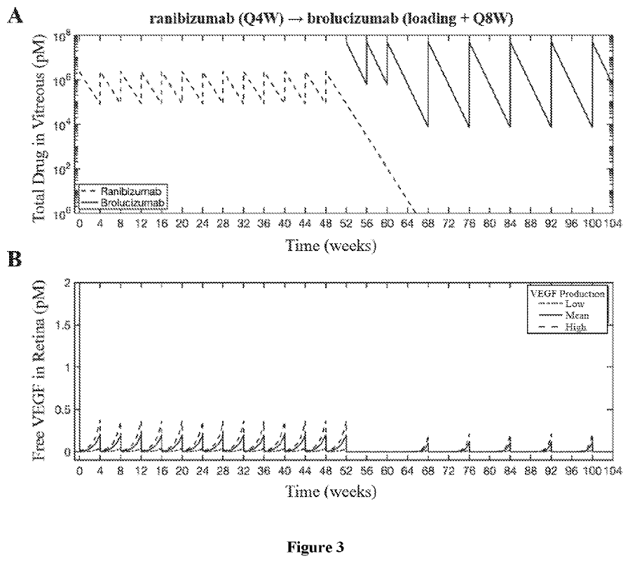

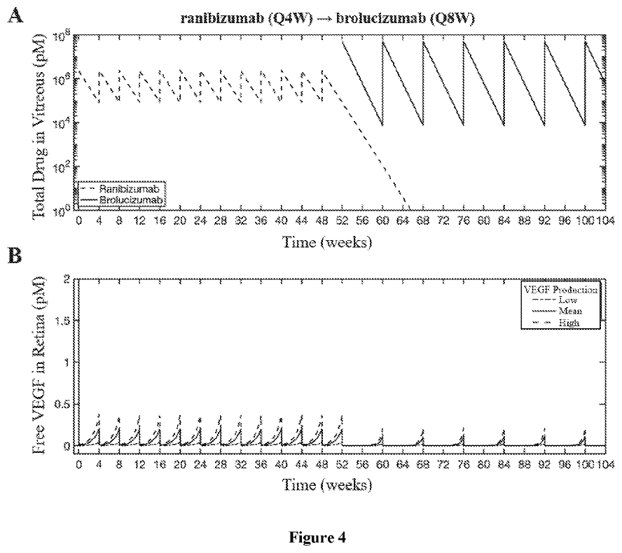

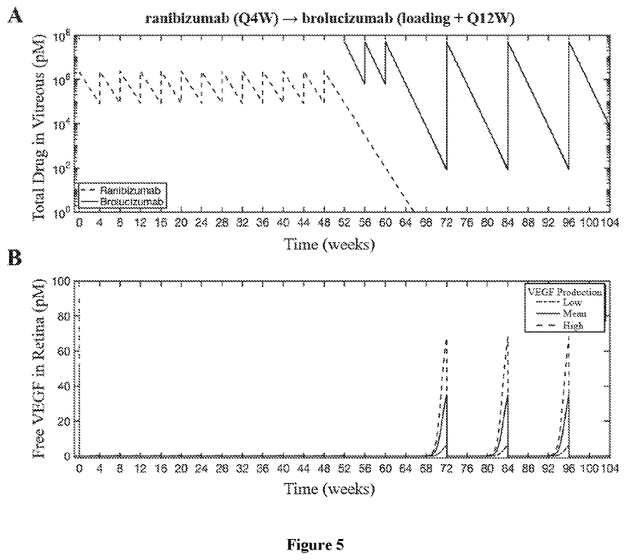

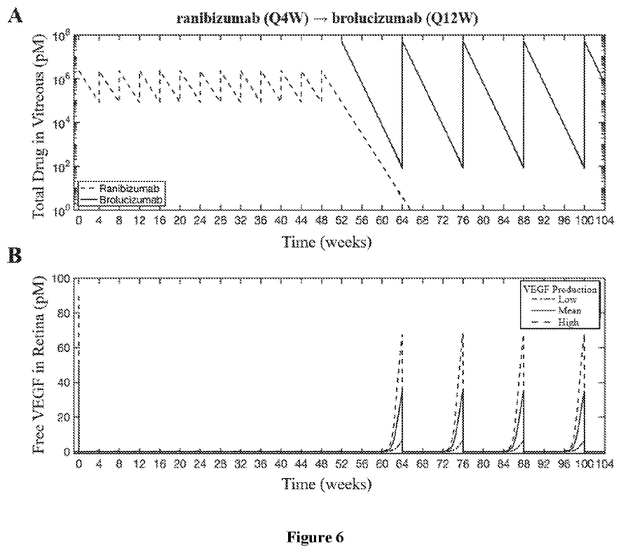

Model simulations were performed for the following scenarios:
(i) A patient that was administered ranibizumab by IVT at a dose of 0.5 mg every 4 weeks (Q4W) between Weeks 0 and 48, and thereafter switched to brolucizumab. Brolucizumab was administered by IVT at a dose of 6.0 mg with a regimen consisting of a loading period (one dose every 4 weeks for the first three months) and a maintenance period (one dose every 8 weeks) between Weeks 52 and 100 (FIG. 3), and
(ii) A patient that was administered ranibizumab by IVT at a dose of 0.5 mg every 4 weeks (Q4W) between Weeks 0 and 48, and thereafter switched to brolucizumab. Brolucizumab was administered by IVT at a dose of 6.0 mg with a regimen consisting of a maintenance period (one dose every 8 weeks) between Weeks 52 and 100, but without the loading doses of brolucizumab (FIG. 4), and
(iii) A patient that was administered ranibizumab by IVT at a dose of 0.5 mg every 4 weeks (Q4W) between Weeks 0 and 48, and thereafter switched to brolucizumab. Brolucizumab was administered by IVT at a dose of 6.0 mg with a regimen consisting of a loading period (one dose every 4 weeks for the first three months) and a maintenance period (one dose every 12 weeks) between Weeks 52 and 96 (FIG. 5), and
(iv) A patient that was administered ranibizumab by IVT at a dose of 0.5 mg every 4 weeks (Q4W) between Weeks 0 and 48, and thereafter switched to brolucizumab. Brolucizumab was administered by IVT at a dose of 6.0 mg with a regimen consisting of a maintenance period (one dose every 12 weeks) between Weeks 52 and 100, but without the loading doses of brolucizumab (FIG. 6).

Free VEGF in the retina was shown to rapidly decrease following the first drug administration and to incompletely recover before each of the subsequent doses (FIGS. 3B, 4B. 5B and 6B). The timing and degree of the recovery for free VEGF in the retina is shown to vary based upon the level of VEGF production in the eye (exemplified as Low, Mean, or High levels of VEGF production), the specific VEGF antagonist administered, and the dosing interval (FIGS. 3B, 4B, 5B and 6B). The inclusion of a loading period for brolucizumab following a switch from prior administration of ranibizumab is observed to yield approximately similar levels of inhibition of free VEGF in the retina as compared to a regimen of brolucizumab without a loading period. This is demonstrated for brolucizumab regimens employing either a Q8W (FIGS. 3 and 4) or Q12W (FIGS. 5 and 6) dosing interval in the maintenance phase.

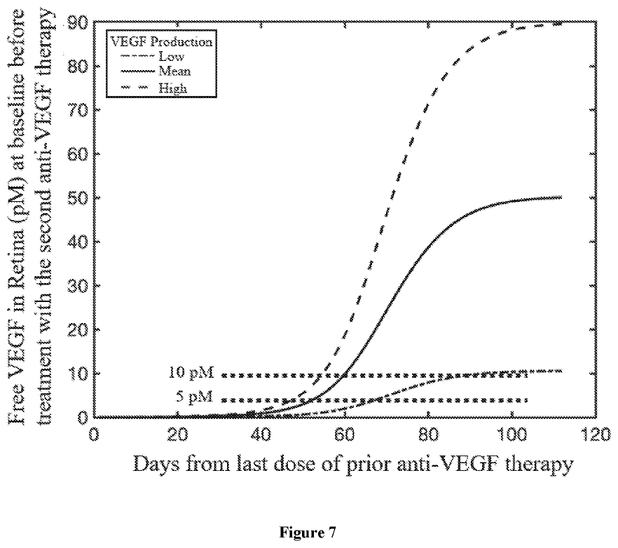

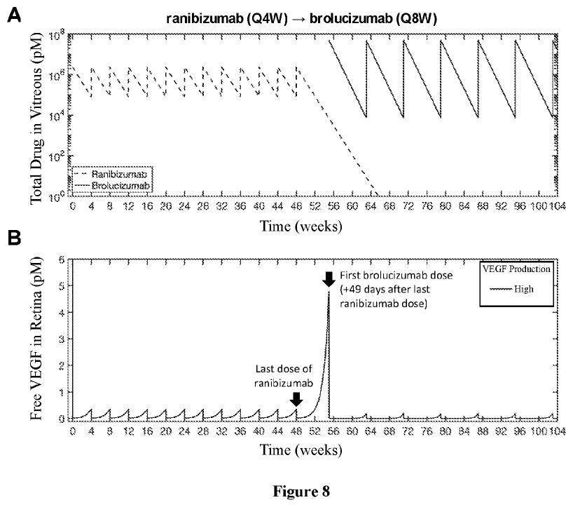

The duration of time, that free VEGF in the retina is maintained below indicated threshold concentrations of 5 pM and 10 pM following the last dose of ranibizumab, was assessed (FIG. 7, Table 5). Ranibizumab was administered by IVT at a dose of 0.5 mg every 4 weeks (Q4W) between Weeks 0 and 48. The timing and degree of the recovery for free VEGF in the retina is shown to vary based upon the level of VEGF production in the eye (exemplified as Low, Mean, or High levels of VEGF production). Based on the findings of FIG. 7 and Table 5, a model simulation as described under 2.1(ii) and in FIG. 4 but with the first dose of brolucizumab at Day 49 following the last dose of ranibizumab and only under the condition of high VEGF production was performed. FIG. 8 illustrates drug switching to brolucizumab under the simulation conditions in which free VEGF in the retina is first observed to surpass the indicated 5 pM threshold following the last dose of ranibizumab (as shown in FIG. 7). This observation suggests that administration of the first dose of brolucizumab may be performed at a date after the next scheduled dose of the prior ranibizumab therapy (28 days) without recovery of free VEGF in the retina beyond the most stringent 5 pM threshold defined to be efficacious. For a patient with high levels of VEGF production, the date of the first dose of brolucizumab can be up to 21 days after the next scheduled dose.

TABLE 5
Duration of free VEGF in retina below thresholds after
the last dose of prior anti-VEGF therapy (days).

| | Prior anti-VEGF therapy: | | Prior anti-VEGF therapy: | | Prior anti-VEGF therapy: | |
|---|---|---|---|---|---|---|
| | ranibizumab | | aflibercept | | brolucizumab | |
| | Free VEGF | Free VEGF | Free VEGF | Free VEGF | Free VEGF | Free VEGF |
| | in retina | in retina | in retina | in retina | in retina | in retina |
| | threshold | threshold | threshold | threshold | threshold | threshold |
| | 5 pM | 10 pM | 5 pM | 10 pM | 5 pM | 10 pM |
| VEGF | Duration of free VEGF in retina below thresholds | | | | | |
| Production | after the last dose of prior anti-VEGF therapy (days) | | | | | |
| Low | 70 | 93 | 111 | 143 | 81 | 97 |
| Mean | 54 | 60 | 92 | 99 | 70 | 73 |
| High | 49 | 54 | 87 | 93 | 67 | 70 |

### 2.2 Aflibercept→Brolucizumab

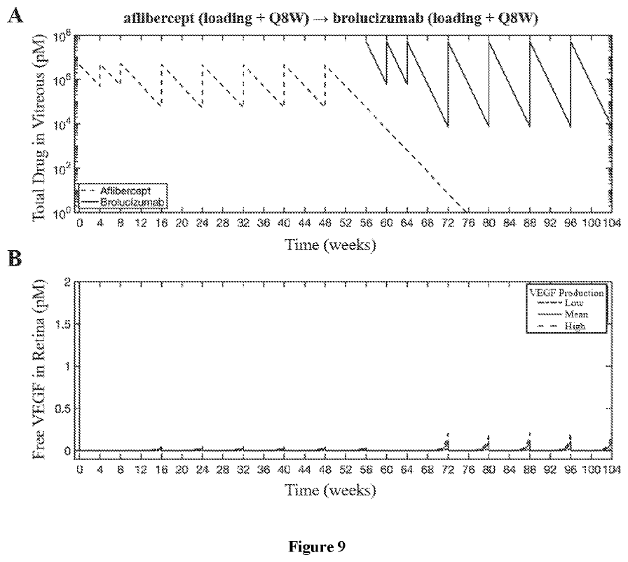

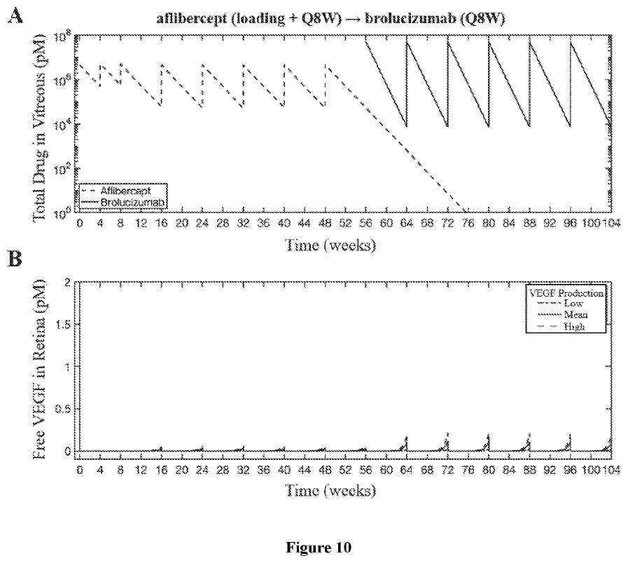

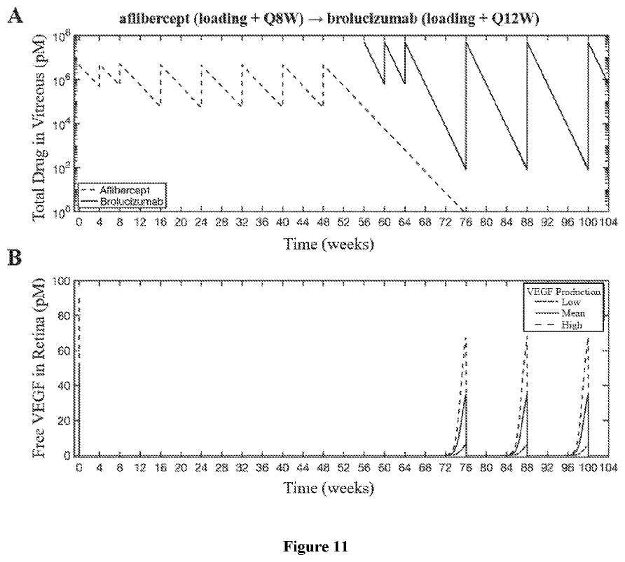

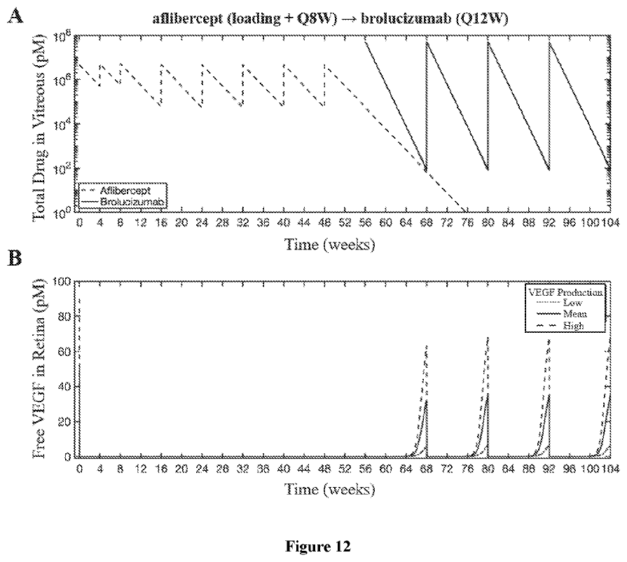

Model simulations were performed for the following scenarios:
(i) A patient that was administered aflibercept by IVT at a dose of 2.0 mg with a regimen consisting of a loading period (one dose every 4 weeks for the first three months) and a maintenance period (one dose every 8 weeks) between Weeks 0 and 48, and thereafter switched to brolucizumab. Brolucizumab was administered by IVT at a dose of 6.0 mg with a regimen consisting of a loading period (one dose every 4 weeks for the first three months) and a maintenance period (one dose every 8 weeks) between Weeks 56 and 96 (FIG. 9), and
(ii) A patient that was administered aflibercept by IVT at a dose of 2.0 mg with a regimen consisting of a loading period (one dose every 4 weeks for the first three months) and a maintenance period (one dose every 8 weeks) between Weeks 0 and 48, and thereafter switched to brolucizumab. Brolucizumab was administered by IVT at a dose of 6.0 mg with a regimen consisting of a maintenance period (one dose every 8 weeks) starting at week 56, but without the loading doses of brolucizumab (FIG. 10), and
(iii) A patient that was administered aflibercept by IVT at a dose of 2.0 mg with a regimen consisting of a loading period (one dose every 4 weeks for the first three months) and a maintenance period (one dose every 8 weeks) between Weeks 0 and 48, and thereafter switched to brolucizumab. Brolucizumab was administered by IVT at a dose of 6.0 mg with a regimen consisting of a loading period (one dose every 4 weeks for the first three months) and a maintenance period (one dose every 12 weeks) (FIG. 11), and
(iv) A patient that was administered aflibercept by IVT at a dose of 2.0 mg with a regimen consisting of a loading period (one dose every 4 weeks for the first three months) and a maintenance period (one dose every 8 weeks) between Weeks 0 and 48, and thereafter switched to brolucizumab. Brolucizumab was administered by IVT at a dose of 6.0 mg with a regimen consisting of a maintenance period (one dose every 12 weeks) between Weeks 56 and 92, but without the loading doses of brolucizumab (FIG. 12).

Free VEGF in the retina was shown to rapidly decrease following the first drug administration and to incompletely recover before each of the subsequent doses (FIGS. 3B, 4B. 5B and 6B). The timing and degree of the recovery for free VEGF in the retina is shown to vary based upon the level of VEGF production in the eye (exemplified as Low, Mean, or High levels of VEGF production), the specific VEGF antagonist administered, and the dosing interval (FIGS. 9B, 10B, 11B and 12B). The inclusion of a loading period for brolucizumab following a switch from prior administration of aflibercept is observed to yield approximately similar levels of inhibition of free VEGF in the retina as compared to a regimen of brolucizumab without a loading period. This is demonstrated for brolucizumab regimens employing either a Q8W (FIGS. 9 and 10) or Q12W (FIGS. 11 and 12) dosing interval in the maintenance phase.

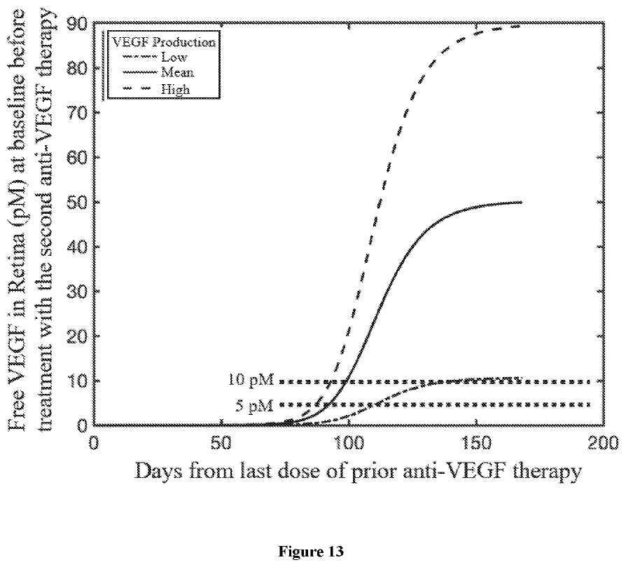

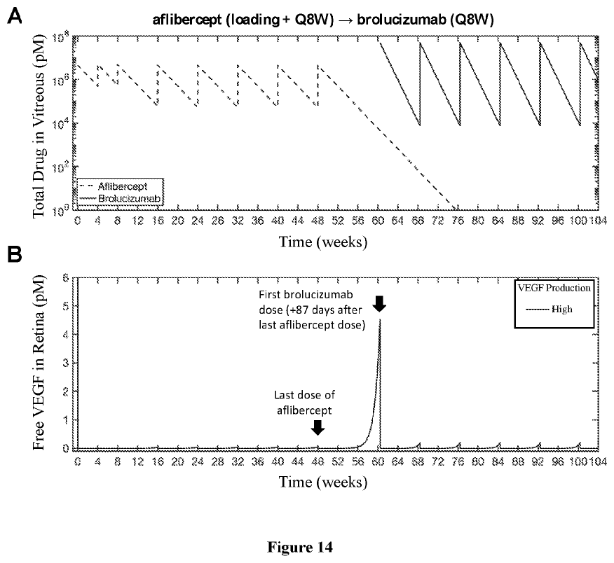

The duration of time, that free VEGF in the retina is maintained below indicated threshold concentrations of 5 pM and 10 pM following the last dose of aflibercept, was assessed (FIG. 13, Table 5). Aflibercept was administered by IVT at a dose of 2.0 mg with a regimen consisting of a loading period (one dose every 4 weeks for the first three months) and a maintenance period (one dose every 8 weeks) between Weeks 0 and 48. The timing and degree of the recovery for free VEGF in the retina is shown to vary based upon the level of VEGF production in the eye (exemplified as Low, Mean, or High levels of VEGF production). Based on the findings of FIG. 13 and Table 5, a model simulation as described under 2.2(ii) and in FIG. 10 but with the first dose of brolucizumab at Day 87 following the last dose of aflibercept and only under the condition of high VEGF production was performed. FIG. 14 illustrates drug switching to brolucizumab under the simulation conditions in which free VEGF in the retina is first observed to surpass the indicated 5 pM threshold following the last dose of aflibercept (as shown in FIG. 13). This observation suggests that administration of the first dose of brolucizumab may be performed at a date after the next scheduled dose of the prior aflibercept therapy (56 days) without recovery of free VEGF in the retina beyond the most stringent 5 pM threshold defined to be efficacious. For a patient with high levels of VEGF production, the date of the first dose of brolucizumab can be up to 31 days after the next scheduled dose.

### 2.3 Brolucizumab→Aflibercept

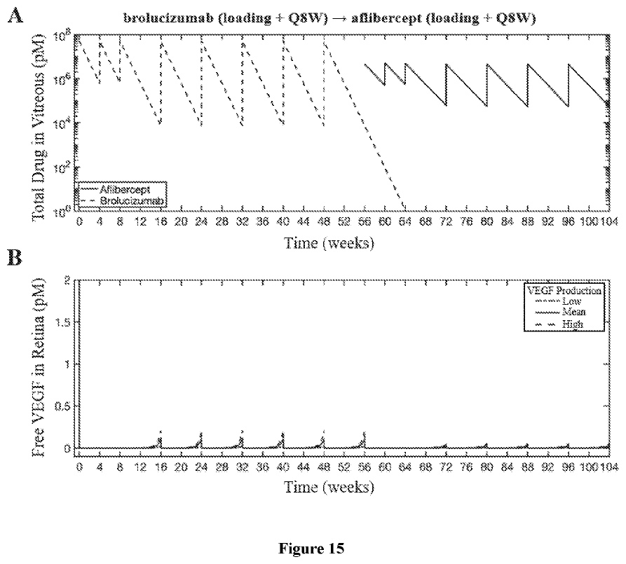

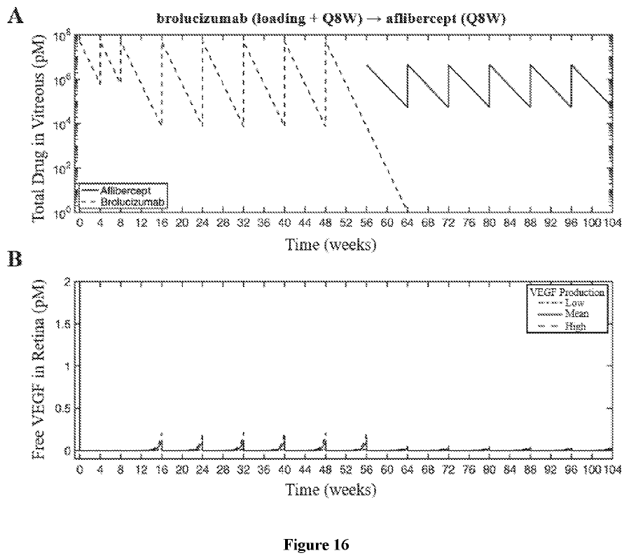

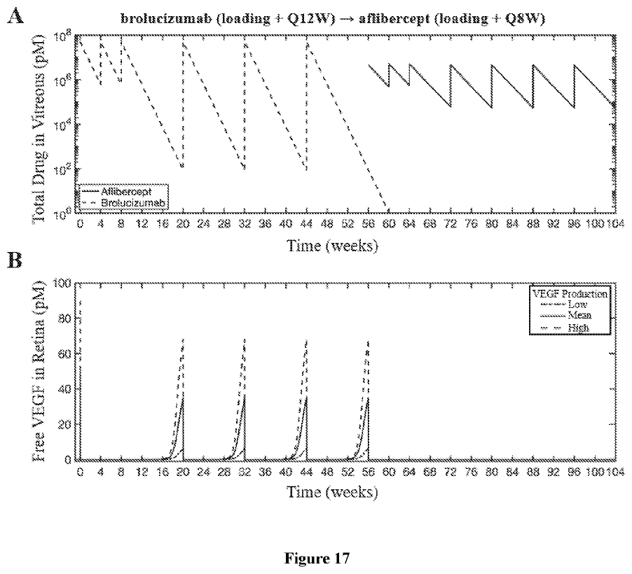

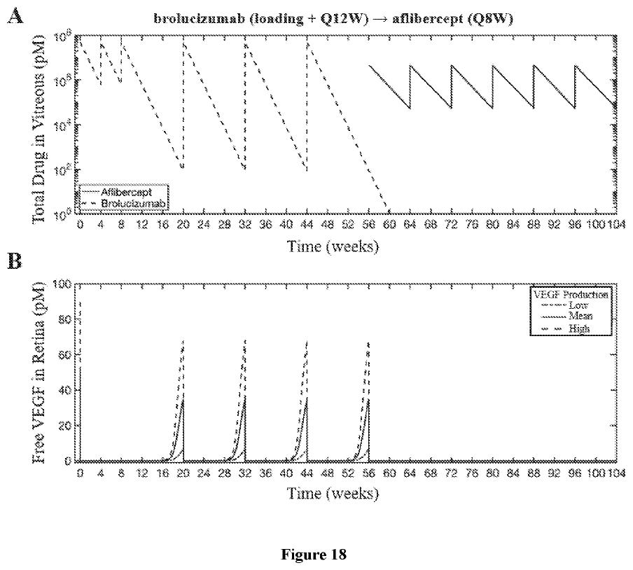

Model simulations were performed for the following scenarios:
(i) A patient that was administered brolucizumab by IVT at a dose of 6.0 mg with a regimen consisting of a loading period (one dose every 4 weeks for the first three months) and a maintenance period (one dose every 8 weeks) between Weeks 0 and 48, and thereafter switched to aflibercept. Aflibercept was administered by IVT at a dose of 2.0 mg with a regimen consisting of a loading period (one dose every 4 weeks for the first three months) and a maintenance period (one dose every 8 weeks) between Weeks 56 and 96 (FIG. 15), and
(ii) A patient that was administered brolucizumab by IVT at a dose of 6.0 mg with a regimen consisting of a loading period (one dose every 4 weeks for the first three months) and a maintenance period (one dose every 8 weeks) between Weeks 0 and 48, and thereafter switched to aflibercept. Aflibercept was administered by IVT at a dose of 2.0 mg with a regimen consisting of a maintenance period (one dose every 8 weeks) starting at Week 56, but without the loading doses of aflibercept (FIG. 16), and
(iii) A patient that was administered brolucizumab by IVT at a dose of 6.0 mg with a regimen consisting of a loading period (one dose every 4 weeks for the first three months) and a maintenance period (one dose every 12 weeks) between Weeks 0 and 44, and thereafter switched to aflibercept. Aflibercept was administered by IVT at a dose of 2.0 mg with a regimen consisting of a loading period (one dose every 4 weeks for the first three months) and a maintenance period (one dose every 8 weeks) (FIG. 17), and
(iv) A patient that was administered brolucizumab by IVT at a dose of 6.0 mg with a regimen consisting of a loading period (one dose every 4 weeks for the first three months) and a maintenance period (one dose every 12 weeks) between Weeks 0 and 42, and thereafter switched to aflibercept. Aflibercept was administered by IVT at a dose of 2.0 mg with a regimen consisting of a maintenance period (one dose every 8 weeks), but without the loading doses of aflibercept (FIG. 18).

Free VEGF in the retina was shown to rapidly decrease following the first drug administration and to incompletely recover before each of the subsequent doses (FIGS. 15B, 16B. 17B and 18B). The timing and degree of the recovery for free VEGF in the retina is shown to vary based upon the level of VEGF production in the eye (exemplified as Low, Mean, or High levels of VEGF production), the specific VEGF antagonist administered, and the dosing interval (FIGS. 15B, 16B. 17B and 18B). The inclusion of a loading period for aflibercept following a switch from prior administration of brolucizumab is observed to yield approximately similar levels of inhibition of free VEGF in the retina as compared to a regimen of aflibercept without a loading period. This is demonstrated following prior brolucizumab regimens consisting of a loading phase or no loading phase and a Q8W or Q12W dosing interval in the maintenance phase (FIGS. 15, 16, 17, and 18).

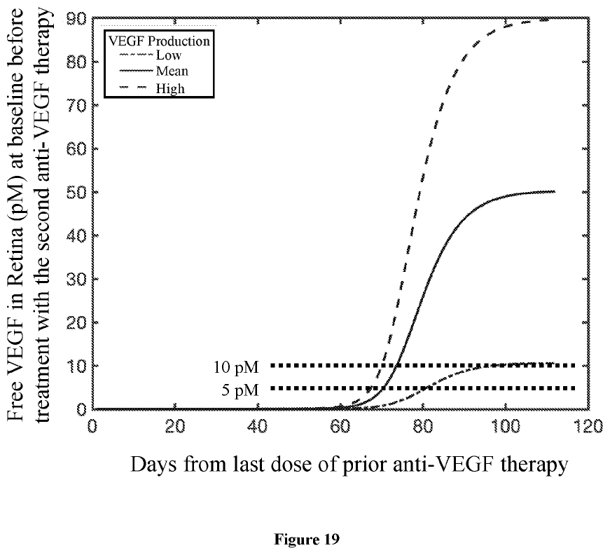

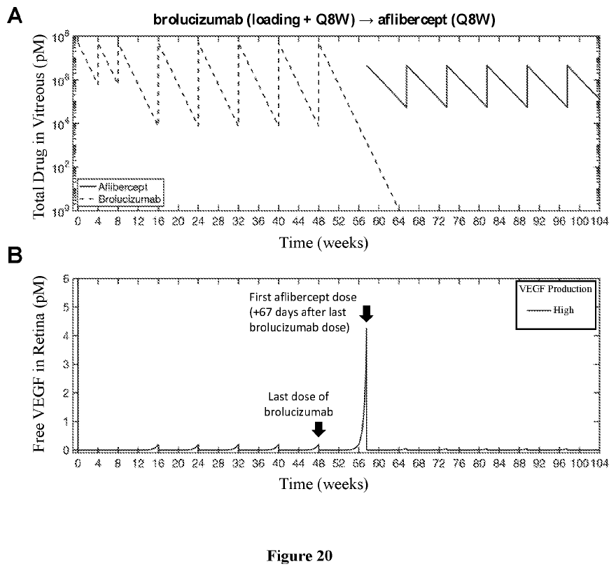

The duration of time, that free VEGF in the retina is maintained below indicated threshold concentrations of 5 pM and 10 pM following the last dose of brolucizumab, was assessed (FIG. 19, Table 5). Brolucizumab was administered by IVT at a dose of 6.0 mg with a regimen consisting of a loading period (one dose every 4 weeks for the first three months) and a maintenance period (one dose every 8 weeks) between Weeks 0 and 48. The timing and degree of the recovery for free VEGF in the retina is shown to vary based upon the level of VEGF production in the eye (exemplified as Low, Mean, or High levels of VEGF production). Based on the findings of FIG. 19 and Table 5, a model simulation as described under 2.3(ii) and in FIG. 16 but with the first dose of aflibercept at Day 67 following the last dose of brolucizumab and only under the condition of high VEGF production. FIG. 20 illustrates drug switching to aflibercept under the simulation conditions in which free VEGF in the retina is first observed to surpass the indicated 5 pM threshold following the last dose of brolucizumab (as shown in FIG. 19).

3. Model Simulations of Drug Concentration Over Time and the Level of Free VEGF in a Patient Treated with Brolucizumab According to q8w or q12w Dosing Regimen

Model simulations were performed for the drug concentration over time (PK) within the vitreous compartment of the eye and the level of free VEGF in the retina compartment of the eye of a patient with Low, Mean, or High predicted levels of VEGF production, in which a patient was administered brolucizumab according to q8w or q12w dosing regimen (without loading phase consisting of 3 monthly administrations of brolucizumab) for a period of approximately one year.

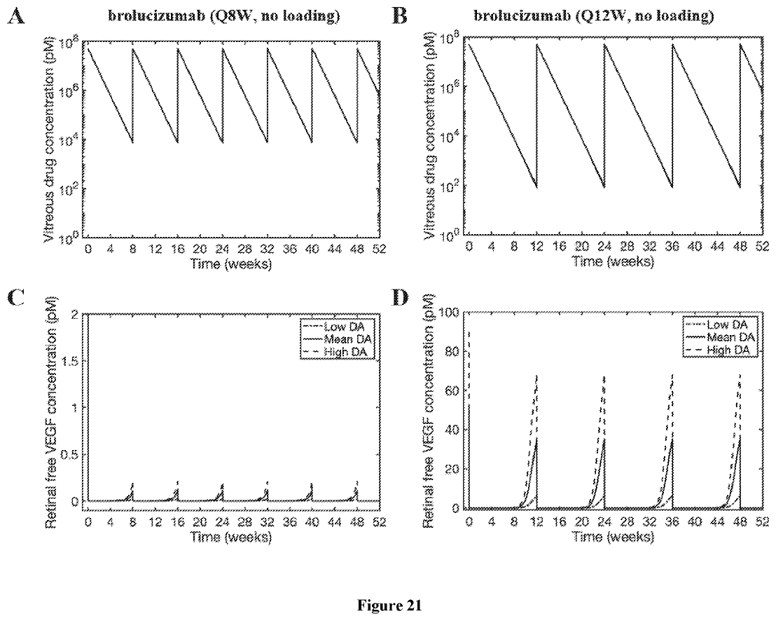

Model simulations were performed for the following scenarios:
(i) A naïve patient that was administered brolucizumab by IVT at a dose of 6.0 mg with a regimen consisting of a loading period (one dose every 4 weeks for the first three months) and a maintenance period (one dose every 8 weeks) between Weeks 0 and 52 (FIG. 2A), and
(ii) A naïve patient that was administered brolucizumab by IVT at a dose of 6.0 mg with a regimen consisting of one dose every 8 weeks (a maintenance period) between Weeks 0 and 52, but without the loading doses of brolucizumab (e.g., omitting 3 monthly doses of brolucizumab) (FIG. 21A), and
(iii) A naïve patient that was administered brolucizumab by IVT at a dose of 6.0 mg with a regimen consisting of a loading period (one dose every 4 weeks for the first three months) and a maintenance period (one dose every 12 weeks) between Weeks 0 and 52 (FIG. 2B), and
(iv) A naïve patient that was administered brolucizumab by IVT at a dose of 6.0 mg with a regimen consisting of one dose every 12 weeks (a maintenance period) between Weeks 0 and 52, but without the loading doses of brolucizumab (e.g., omitting 3 monthly doses of brolucizumab) (FIG. 21B).

Free VEGF in the retina was shown to rapidly decrease following the first drug administration and to incompletely recover before each of the subsequent doses (FIGS. 2C, 2D, 21C and 21D). The timing and degree of the recovery for free VEGF in the retina is shown to vary based upon the level of VEGF production in the eye (exemplified as Low, Mean, or High levels of VEGF production), and the dosing interval (FIGS. 2C, 2D, 21C and 21D). The inclusion of a loading period for brolucizumab is observed to yield approximately similar levels of inhibition of free VEGF in the retina as compared to a regimen of brolucizumab without a loading period. This is demonstrated for brolucizumab regimens employing either a Q8W (FIGS. 2C and 21C) or Q12W (FIGS. 2D and 21D) dosing interval in the maintenance phase.

The duration of time, that free VEGF in the retina is maintained below indicated threshold concentrations of 5 pM for ranibizumab, aflibercept, brolucizumab (loading +q8w), brolucizumab (loading +q12w), brolucizumab (q8w) and brolucizumab (q12w) were assessed (Table 6):
(1) Ranibizumab was administered by IVT at a dose of 0.5 mg every 4 weeks (Q4W) between Weeks 0 and 52.
(2) Aflibercept was administered by IVT at a dose of 2.0 mg with a regimen consisting of a loading period (one dose every 4 weeks for the first three months) and a maintenance period (one dose every 8 weeks) between Weeks 0 and 52.
(3) Brolucizumab was administered by IVT at a dose of 6.0 mg with a regimen consisting of a loading period (one dose every 4 weeks for the first three months) and a maintenance period (one dose every 8 weeks) between Weeks 0 and 52.
(4) Brolucizumab was administered by IVT at a dose of 6.0 mg with a regimen consisting of a maintenance period (one dose every 8 weeks) between Weeks 0 and 52.
(5) Brolucizumab was administered by IVT at a dose of 6.0 mg with a regimen consisting of a loading period (one dose every 4 weeks for the first three months) and a maintenance period (one dose every 12 weeks) between Weeks 0 and 52.
(6) Brolucizumab was administered by IVT at a dose of 6.0 mg with a regimen consisting of a maintenance period (one dose every 12 weeks) between Weeks 0 and 52.
   The timing and degree of the recovery for free VEGF in the retina is shown to vary based upon the level of VEGF production in the eye (exemplified as Low, Mean, or High levels of VEGF production). Based on the findings of FIG. 21 and Table 6, low disease activity (DA) patients (patients with low level of VEGF production) are suited for a Q12w maintenance phase, whereas high DA patients (patients with high level of VEGF production) might benefit from a Q8w maintenance regimen.

TABLE 6
Duration below suppression threshold.

| | Duration below 5 pM rVEGF concentration |
|---|---|
| Brolucizumab | 100.0% (364 of 364 days) for all DA levels |
| (loading + Q8w) | |
| Brolucizumab | 97.8% (356 of 364 days) for low DA |
| (loading + Q12w) | 89.0% (324 of 364 days) for mean DA |
| | 86.5% (315 of 364 days) for high DA |
| Brolucizumab (Q8w) | 100.0% (364 of 364 days) for all DA levels |
| Brolucizumab (Q12w) | 97.0% (353 of 364 days) for low DA |
| | 85.4% (311 of 364 days) for mean DA |
| | 82.1% (299 of 364 days) for high DA |

### Example 2: A Clinical Study

A clinical study was designed to evaluate the efficacy and safety of two different brolucizumab regimens for pretreated patients with neovascular age-related macular degeneration (nAMD). The study investigates whether insufficiently anti-VEGF pretreated nAMD patients (i.e. residual or recurrent fluid present while on an administration interval, e.g., an injection interval, between ≥6 and ≤10 weeks), need loading with three monthly brolucizumab 6 mg injections or whether they can immediately receive brolucizumab treatment every 12 weeks.

The study is a 52-week, two arm, randomized, open-label, multicenter study assessing the efficacy and safety of two different brolucizumab 6 mg dosing regimens for patients with suboptimal anatomically controlled nAMD. The primary objective of this study is to demonstrate that brolucizumab 6 mg with one (initial) injection followed by treatment every 12 weeks is non-inferior to brolucizumab 6 mg with three monthly loading injections followed by treatment every 12 weeks. The primary endpoint will be assessed as the mean change in BCVA from baseline to mean of visits at week 40 to 52.

Study Population:

The study population is male and female patients≥50 years old diagnosed with active choroidal neovascularization (CNV) secondary to AMD, treated previously for this disease and able to comply with study or follow-up procedures.

Key Inclusion Criteria:

Subjects eligible for inclusion in this study must meet all of the following criteria:
1. Signed informed consent must be obtained prior to participation in the study.
2. Male or female patients, ≥50 years of age at screening.

Study Eye:
3. Active choroidal neovascularization (CNV) secondary to AMD that affects the central subfield, including retinal angiomatous proliferation (RAP) with a CNV component, confirmed by presence of active leakage from CNV seen by fluorescein angiography and sequelae of CNV, e.g. pigment epithelial detachment (PED), subretinal or sub-retinal pigment epithelium (sub-RPE) hemorrhage, blocked fluorescence, macular edema (intraretinal fluid (IRF) and/or subretinal fluid (SRF) and/or sub-retinal pigment epithelium (sub-RPE) fluid that affects the central subfield, as seen by SD-OCT) as confirmed by central reading center.
4. Previous treatment with any licensed anti-VEGF drug for 6 months in a q6w to ≤q10w injection interval with residual or recurrent fluid (intraretinal fluid (IRF) and/or subretinal fluid (SRF)) that affects the central subfield, as seen by SD-OCT, despite continuous licensed anti-VEGF treatment at baseline. In total, patients should not be pretreated for more than 36 months.
5. BCVA score must be 83 and 38 letters at 4 meters starting distance using Early Treatment Diabetic Retinopathy Study (ETDRS)-like visual acuity charts (approximately Snellen equivalent of 20/25 and 20/200), at both screening and baseline.

Key Exclusion Criteria:

Subjects meeting any of the following criteria are not eligible for inclusion in this study.

Ocular Conditions:
Concomitant conditions or ocular disorders in the study eye at screening or baseline which could, in the opinion of the investigator, prevent response to study treatment or may confound interpretation of study results, compromise visual acuity or require planned medical or surgical intervention during the 52-week study period (e.g. structural damage of the fovea, vitreous hemorrhage, retinal vein occlusion, retinal detachment, macular hole, choroidal neovascularization unrelated to age-related macular degeneration, diabetic retinopathy (except mild non-proliferative) and diabetic macular edema).
Treatment with anti-VEGF drugs for >36 months.
Any active intraocular or periocular infection or active intraocular inflammation (e.g. infectious conjunctivitis, keratitis, scleritis, endophthalmitis, infectious blepharitis, uveitis) in study eye at screening or baseline.
Uncontrolled glaucoma in the study eye defined as intraocular pressure (IOP)>25 mmHg on medication as well as untreated glaucoma, or according to investigator's judgment, at screening or baseline.
Presence of amblyopia, amaurosis or ocular disorders in the fellow eye with BCVA<20/200 at screening (except when due to conditions whose surgery may improve VA, e.g. cataract).
Atrophy or fibrosis involving the center of the fovea in the study eye as confirmed by central reading center.
The total area of fibrosis or subretinal blood affecting the foveal center point comprising ≥50% of the lesion area as well as chronic cystic lesions in the study eye as confirmed by central reading center.
Structural damage within 0.5 disc diameter of the center of the macula in the study eye, e.g. vitreomacular traction, epiretinal membrane, scar, laser burn, at the time of screening that in the investigator's opinion could preclude visual function improvement with treatment.

Ocular Treatments:
Previous treatment with investigational drugs in the last 6 months in the study eye.
Previous use of intraocular or periocular steroids (non-topical) in study eye.
Macular laser photocoagulation (focal/grid) or photodynamic therapy in the study eye at any time prior to baseline and peripheral laser photocoagulation in the study eye within 3 months prior to baseline.
Intraocular surgery in the study eye within 3 months prior to baseline.
Vitreoretinal surgery in the study eye at any time prior to baseline.
Aphakia with the absence of posterior capsule in the study eye.

Systemic Conditions or Treatments:
Stroke or myocardial infarction during the 6-month period prior to baseline.
Systemic anti-VEGF therapy during the 3-month period prior to baseline.

Study Treatment:

The treatment used in this study is brolucizumab 6 mg/0.05 mL. Brolucizumab is formulated as a sterile solution aseptically filled in a sterile syringe for single use containing sufficient brolucizumab to deliver a 6 mg dose when administering a volume of 0.05 mL.

Patients who consent undergo screening assessments to evaluate their eligibility based on certain inclusion and exclusion criteria. Subjects who meet all the inclusion and none of the exclusion criteria are randomized 1:1:
1. Brolucizumab 6 mg “loading arm”: 3×4-weekly initial injections followed by an injection every 12 weeks.
2. Brolucizumab 6 mg “non-loading arm”: one initial injection followed by an injection every 12 weeks.

For Both Study Arms:
The assessment of disease activity is performed 8 and 12 weeks after the previous injection.
If disease activity is identified by the investigator for the first time after week 8, patients stay on a q12w dosing, if the decline in BCVA is clinically non-significant (BCVA loss<4 ETDRS letters).
If renewed disease activity is identified or BCVA-loss of ≥5 ETDRS letters appears, patients are assigned to q8w dosing.
One attempt of re-extension to q12w is allowed based on the investigator's judgement if no disease activity (DA) is detected during the subsequent visit(s).
If the patient shows significant DA after the re-extension attempt, injection intervals are fixed to q8w until the end of the study
The disease activity decision should be based on investigator's judgment of visual and/or anatomic outcomes and signs of disease activity (e.g. IRF, SRF, hemorrhage, leakage, etc.).

Efficacy (Disease Activity Assessment)

The following assessments are performed to evaluate the effect of brolucizumab on visual function retinal structure and vascular leakage:
Best-corrected visual acuity with ETDRS-like charts at 4 meters;
Anatomical retinal evaluation of SD-OCT images;
Color Fundus Photography and vascular leakage evaluation by Fluorescein angiography.

All efficacy assessments should be performed prior to any administration of study treatment and/or rescue medication.

Visual Acuity

Visual acuity (VA) is assessed in the study eye at every study visit and in the fellow eye at the screening and week 52/EOS visits using best correction determined from protocol refraction (BCVA). BCVA measurements will be taken in a sitting position using ETDRS-like visual acuity testing charts at an initial testing distance of 4 meters.

SD-Optical Coherence Tomography

Spectral Domain Optical Coherence Tomography (SD-OCT) images are obtained and assessed in the study eye at every study visit and in the fellow eye at the screening and week 52/EOS visits. The central reading center (CRC) checks screening images (SD-OCT, FP, FA) for eligibility of patients within 72 hours of image upload. Only after patients are deemed eligible by the CRC, they may be randomized if all other in/exclusion criteria are met.

SD-OCT assessments should be performed after BCVA assessment and prior to any study drug administration.

In addition to the standard SD-OCT assessment, OCT angiography can be used at screening and EOS/week 52 visits.

Color Fundus Photography and Fluorescein Angiography

Color fundus photography (CFP) and fluorescein angiography (FA) will be performed in both eyes at the screening and week 52/EOS visits. CFP and FA may be performed at other visits at the investigator's discretion. For the purpose of screening, FA images from a previous routine evaluation may be used as long as FA was performed within 7 days of the screening visit.

1-2. (canceled)

3. A method for treating ocular disease in a patient, the method comprising administering to the patient an initial dose of a VEGF antagonist followed by one or more additional doses of the VEGF antagonist, wherein the one or more additional doses of the VEGF antagonist are administered at least 8 weeks after the initial dose and each of the one or more additional doses after the initial dose are administered in an administration interval of at least 8 weeks.

4. (canceled)

5. A method for treating ocular disease in a patient, the method comprising administering to the patient one initial dose of a VEGF antagonist followed by a maintenance regimen of additional doses of the VEGF antagonist administered in an administration interval of at least 8 weeks.

6. The method of claim 5, wherein the maintenance regimen of the VEGF antagonist consists of 2, 3, 4, 5, 6 or more doses administered in an administration interval of at least 8 weeks.

7. The method of claim 5, wherein the method does not comprise administering to the patient one or more additional doses of the VEGF antagonist in an administration interval according to a loading regimen of the VEGF antagonist for the treatment of the ocular disease.

8. The method of claim 7, wherein the loading regimen of the VEGF antagonist consists of 2, 3, 4, 5, or 6 doses of the VEGF antagonist administered at q4w or q6w intervals.

9. The method of claim 3 or 5, wherein the initial dose of the VEGF antagonist is followed by one or more doses of the VEGF antagonist in an administration interval as individualized by a physician based on a disease activity assessment and/or in an administration interval between >8 and <24 weeks.

10. The method of claim 3 or 5, wherein the initial dose of the VEGF antagonist is followed by administering to the patient one or more doses of the VEGF antagonist once every 8 weeks (q8w regimen) or once every 12 weeks (q12w regimen) and/or as individualized by a physician based on a disease activity assessment.

11. The method of claim 10, further comprising assessing the patient for ocular disease activity before or after administering every q8w or q12w dose of the VEGF antagonist.

12. The method of claim 3 or 5, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

13. The method of claim 11, wherein if presence of ocular disease activity is identified after a q12w dose of the VEGF antagonist, the patient is switched to a q8w regimen of the VEGF antagonist.

14. The method of claim 13, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT),
(iv) new or persistent or recurrent Intraretinal Cysts (IRC) and/or Intraretinal Fluid (IRF) and/or Subretinal Fluid (SRF).

15. (canceled)

16. The method of claim 3 or 5, wherein the ocular disease is selected from the list consisting of abnormal angiogenesis, choroidal neovascularization (CNV), retinal vascular permeability, retinal edema, diabetic retinopathy (e.g., proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

17-18. (canceled)

19. The method of claim 3 or 5, wherein the VEGF antagonist is an anti-VEGF antibody, a single chain antibody (scFv), or a Fab fragment.

20. The method of claim 3 or 5, wherein the VEGF antagonist is an anti-VEGF antibody comprising the sequence of SEQ ID NO: 3 or brolucizumab.

21. (canceled)

22. The method of claim 3 or 5, wherein the VEGF antagonist is administered by an intravitreal injection.

23. The method of claim 3 or 5, wherein the dose of the VEGF antagonist is from about 3 mg to about 6 mg.

24. (canceled)

25. The method of claim 3 or 5, wherein the patient is a naive patient.

26. The method of claim 3, wherein the patient is a pretreated with one or more doses of a VEGF antagonist different from the VEGF antagonist.

27. The method of claim 26, wherein the patient was pretreated with a VEGF antagonist selected from the group consisting of aflibercept, ranibizumab, faricimab, conbercept and abicipar.

28-66. (canceled)

67. A method for treating ocular disease in a patient pretreated with one or more doses of a VEGF antagonist B, the method comprising administering to the patient an initial dose of a VEGF antagonist A followed by one or more additional doses of the VEGF antagonist A in an administration interval of no less than about 8 weeks, and/or as individualized by a physician based on a disease activity assessment.

68. A method for treating ocular disease in a patient pretreated with one or more doses of a VEGF antagonist B, the method comprising administering to the patient an initial dose of a VEGF antagonist A followed by one or more additional doses of the VEGF antagonist A in an administration interval according to a maintenance regimen of the VEGF antagonist A for the treatment of the ocular disease.

69. The method of claim 67 or 68, wherein the method comprises discontinuing treatment with the VEGF antagonist B.

70. The method of claim 67 or 68, wherein the VEGF antagonist A is administered in replacement of the VEGF antagonist B and no additional or alternative VEGF antagonists are administered to the patient during the administration of the VEGF antagonist A.

71. The method of claim 67 or 68, wherein the patient pretreated with one or more doses of the VEGF antagonist B has suboptimal anatomically controlled ocular disease.

72. The method of claim 67 or 68, wherein presence of ocular disease activity was identified in the patient pretreated with one or more doses of the VEGF antagonist B.

73. The method of claim 67 or 68, wherein presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT), and
(iv) new or persistent or recurrent Intraretinal Cysts (IRC) and/or Intraretinal Fluid (IRF) and/or Subretinal Fluid (SRF).

74. The method of claim 3, wherein the patient was intolerant to the treatment with the VEGF antagonist B.

75. The method of claim 74, wherein the patient was pretreated with the VEGF antagonist B for at least 3 months or longer.

76. The method of claim 67, wherein the VEGF antagonist B was administered to the patient in an administration interval between about >4 and about <12 weeks.

77. The method of claim 67, wherein the initial dose of the VEGF antagonist A is administered to the patient between about >4 and about <12 weeks after the last dose of the VEGF antagonist B was administered to the patient.

78. The method of claim 67, wherein the initial dose of the VEGF antagonist A is followed by one or more doses of the VEGF antagonist A in an administration interval as individualized by a physician based on a disease activity assessment and/or in an administration interval between about >8 and about <24 weeks.

79. The method of claim 78, wherein the initial dose of the VEGF antagonist A is followed by administering to the patient one or more doses of the VEGF antagonist A once every 8 weeks (q8w regimen) or once every 12 weeks (q12w regimen) and/or as individualized by a physician based on a disease activity assessment.

80. The method of claim 79, further comprising assessing the patient for ocular disease activity before or after administering every q8w or q12w dose of the VEGF antagonist A.

81. The method of claim 67, wherein the disease activity is assessed based on one or more of the following:
(i) best corrected visual acuity (BCVA),
(ii) visual acuity (VA),
(iii) central subfield thickness (CSFT), and/or
(iv) presence of intraretinal cysts/fluid.

82. The method of claim 80, wherein if presence of ocular disease activity is identified after a q12w dose of the VEGF antagonist A, the patient is switched to a q8w regimen of the VEGF antagonist A.

83. The method of claim 82, wherein the presence of ocular disease activity includes one or more of the following:
(i) decrease in Best Corrected Visual Acuity (BCVA),
(ii) decrease in Visual Acuity (VA),
(iii) increase or lack of reduction in Central Subfield Thickness (CSFT), (iv) new or persistent or recurrent Intraretinal Cysts (IRC) and/or Intraretinal Fluid (IRF) and/or Subretinal Fluid (SRF).

84. (canceled)

85. The method of claim 67, wherein the ocular disease is selected from the list consisting of abnormal angiogenesis, choroidal neovascularization (CNV), retinal vascular permeability, retinal edema, diabetic retinopathy (particularly proliferative diabetic retinopathy (PDR) and non-proliferative diabetic retinopathy (NPDR)), macular edema (ME), diabetic macular edema (DME), neovascular (exudative) age-related macular degeneration (nAMD), choroidal neovascularization (CNV) associated with nAMD, sequela associated with retinal ischemia, Retinal Vein Occlusion (RVO), Central Retinal Vein Occlusion (CRVO), Branch Retinal Vein Occlusion (BRVO), macular edema following retinal vein occlusion, and posterior segment neovascularization.

86-91. (canceled)

92. The method of claim 67, wherein the VEGF antagonist A is different from the VEGF antagonist B.

93. The method of claim 67, wherein the VEGF antagonist A is an anti-VEGF antibody, a single chain antibody (scFv), or a Fab fragment.

94. The method of claim 67, wherein the VEGF antagonist A is an anti-VEGF antibody comprising the sequence of SEQ ID NO: 3 or brolucizumab.

95. (canceled)

96. The method of claim 67, wherein the VEGF antagonist A is administered by an intravitreal injection.

97. The method of claim 67, wherein the dose of the VEGF antagonist A is from about 3 mg to about 6 mg.

98. (canceled)

99. The method of claim 67, wherein the VEGF antagonist B is selected from the group consisting of aflibercept, ranibizumab, faricimab, conbercept and abicipar.

100-146. (canceled)
